(19) United States
(12) Patent Application Publication
(10) Pub. No.: US 2015/0299125 A1
(43) Pub. Date: Oct. 22, 2015

(54) PRODRUG BIPYRIDYLAMINOPYRIDINES AS SYK INHIBITORS

(71) Applicants: Andrew M. HAIDLE, Boston, MA (US); Brandon CASH, Boston, MA (US); James P. JEWELL, Boston, MA (US); Kerrie SPENCER, Boston, MA (US); Michael D. ALTMAN, Boston, MA (US); Ryan D. OTTE, Boston, MA (US); Alan B. NORTHRUP, Boston, MA (US); MERCK SHARP & DOHME CORP., Rahway, NJ (US)

(72) Inventors: Andrew M. Haidle, Cambridge, MA (US); Brandon Cash, Framingham, MA (US); James P. Jewell, Newton, MA (US); Kerrie Spencer, Woonsocket, RI (US); Michael D. Altman, Needham, MA (US); Ryan D. Otte, Natick, MA (US); Alan B. Northrup, Reading, MA (US)

(73) Assignee: Merck Sharp & Dohme Corp., Rahway, NJ (US)

(21) Appl. No.: 14/440,517

(22) PCT Filed: Nov. 4, 2013
(86) PCT No.: PCT/US13/68189

§ 371 Date: May 4, 2015

Related U.S. Application Data
(60) Provisional application No. 61/723,571, filed on Nov. 7, 2012.

(51) Int. Cl. C07D 213/74; C07D 401/14; C07D 405/14

(52) U.S. Cl. CPC C07D 213/74; C07D 405/14; C07D 401/14

(57) ABSTRACT

The present invention provides compounds of Formula (I), which are prodrugs of trans-4-[(1R)-(6-{[4-(difluoromethyl)pyridin-2-yl]amino}-4-methyl-2,3′-bipyridin-6′-yl)-1-hydroxyethyl]cyclohexanecarboxylic acid, a potent inhibitor of Syk. The compounds are useful in the treatment and prevention of diseases mediated by the enzyme, such as asthma, COPD, rheumatoid arthritis and cancer.

### BACKGROUND OF THE INVENTION

Spleen Tyrosine Kinase (Syk) is a protein tyrosine kinase which has been described as a key mediator of immunoreceptor signalling in a host of inflammatory cells including mast cells, B-cells, macrophages and neutrophils. These immunoreceptors, including Fc receptors and the B-cell receptor, are important for both allergic diseases and antibody-mediated autoimmune diseases and thus pharmacologically interfering with Syk could conceivably treat these disorders.

Allergic rhinitis and asthma are diseases associated with hypersensitivity reactions and inflammatory events involving a multitude of cell types including mast cells, eosinophils, T cells and dendritic cells. Following exposure to allergen, high affinity immunoglobulin receptors for IgE and IgG become cross-linked and activate downstream processes in mast cells and other cell types leading to the release of pro-inflammatory mediators and airway spasmogens. In the mast cell, for example, IgE receptor cross-linking by allergen leads to release of mediators including histamine from pre-formed granules, as well as the synthesis and release of newly synthesized lipid mediators including prostaglandins and leukotrienes.

Syk kinase is a non-receptor linked tyrosine kinase which is important in transducing the downstream cellular signals associated with cross-linking FcₑₚₛᵢₗₒₙR1 and or FcₑₚₛᵢₗₒₙR1 receptors, and is positioned early in the signalling cascade. In mast cells, for example, the early sequence of FcₑₚₛᵢₗₒₙR1 signalling following allergen cross-linking of receptor-IgE complexes involves first Lyn (a Src family tyrosine kinase) and then Syk. Inhibitors of Syk activity would therefore be expected to inhibit all downstream signalling cascades thereby alleviating the immediate allergic response and adverse events initiated by the release of pro-inflammatory mediators and spasmogens (Wong et al 2004, Expert Opin. Investig. Drugs (2004) 13 (7) 743-762).

Recently, it has been shown that the Syk kinase inhibitor R112 (Rigel), dosed intranasally in a phase I/II study for the treatment of allergic rhinitis, gave a statistically significant decrease in PGD₂, a key immune mediator that is highly correlated with improvements in allergic rhinorrhea, as well as being safe across a range of indicators, thus providing the first evidence for the clinical safety and efficacy of a topical Syk kinase inhibitor. (Meltzer, Eli O.; Berkowitz, Robert B.; Grossbard, Elliott B, Journal of Allergy and Clinical Immunology (2005), 115(4), 791-796). In a more recent phase II clinical trial for allergic rhinitis (Clinical Trials.gov Identifier NCT0015089), R112 was shown as having a lack of efficacy versus placebo.

Rheumatoid Arthritis (RA) is an auto-immune disease affecting approximately 1% of the population. It is characterised by inflammation of articular joints leading to debilitating destruction of bone and cartilage. Recent clinical studies with Rituximab, which causes a reversible B cell depletion, (J. C. W. Edwards et al 2004, New Eng. J. Med. 350: 2572-2581) have shown that targeting B cell function is an appropriate therapeutic strategy in auto-immune diseases such as RA. Clinical benefit correlates with a reduction in auto-reactive antibodies (or Rheumatoid Factor) and these studies suggest that B cell function and indeed auto-antibody production are central to the ongoing pathology in the disease.

Studies using cells from mice deficient in the Spleen Tyrosine Kinase (Syk) have demonstrated a non-redundant role of this kinase in B cell function. The deficiency in Syk is characterized by a block in B cell development (M. Turner et al 1995 Nature 379: 298-302 and Cheng et al 1995, Nature 378: 303-306). These studies, along with studies on mature B cells deficient in Syk (Kurasaki et al 2000, Immunol. Rev. 176:19-29), demonstrate that Syk is required for the differentiation and activation of B cells. Hence, inhibition of Syk in RA patients is likely to block B cell function and thereby reduce Rheumatoid Factor production. In addition to the role of Syk in B cell function, and of further relevance to the treatment of RA, is the requirement for Syk activity in Fc receptor (FcR) signalling. FcR activation by immune complexes in RA has been suggested to contribute to the release of multiple pro-inflammatory mediators.

The present invention relates to novel compounds, which are prodrugs of inhibitors of Syk kinase activity. These compounds therefore have potential therapeutic benefit in the treatment of disorders associated with inappropriate Syk activity, in particular in the treatment and prevention of disease states mediated by Syk. Such disease states may include inflammatory, allergic and autoimmune diseases, for example, asthma, chronic obstructive pulmonary disease (COPD), adult respiratory distress syndrome (ARDS), ulcerative colitis, Crohns disease, bronchitis, dermatitis, allergic rhinitis, psoriasis, scleroderma, urticaria, rheumatoid arthritis, idiopathic thrombocytopenic purpura (ITP), multiple sclerosis, cancer, HIV and lupus.

### SUMMARY OF THE INVENTION

The present invention provides compounds of Formula (I), which are prodrugs of trans-4-[(1R)-(6-{[4-(difluoromethyl)pyridin-2-yl]amino}-4-methyl-2,3′-bipyridin-6′-yl)-1-hydroxyethyl]cyclohexanecarboxylic acid, a potent inhibitor of Syk. In certain embodiments, the compounds of Formula (I), by themselves, inhibit Syk. The present invention also provides pharmaceutical compositions containing such compounds. As Syk inhibitors or prodrugs thereof, compounds of the present invention are useful in the treatment and prevention of diseases and disorders mediated by the Syk protein; such diseases and disorders include, but are not limited to, asthma, COPD, rheumatoid arthritis, cancer and idiopathic thrombocytopenic purpura.

### DETAILED DESCRIPTION OF THE INVENTION

### Definitions

In the application various terms are as defined below unless specified otherwise:

“Alkyl” refers to a straight- or branched-chain hydrocarbon radical having the specified number of carbon atoms. Examples of “alkyl” include, but are not limited to, methyl, ethyl, n-propyl, isopropyl, n-butyl, isobutyl, t-butyl, n-pentyl, isopentyl, and the like.

“Alkylene” refers to an alkyl group, as defined above, wherein one the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —C(H)(CH₃)CH₂CH₂—, —C(H)(CH₃)₂—, and —CH₂C(H)(CH₃)CH₂—.

“Aryl” refers to an aromatic monocyclic or multicyclic ring system comprising about 6 to about 14 carbon atoms, preferably about 6 to about 10 carbon atoms. Examples include phenyl and naphthyl.

“Carbocycle” refers to a non-aromatic saturated or partially unsaturated monocyclic ring in which all ring atoms are carbon, and the ring being isolated or fused (including ortho-fused, spiro-fused and bridged) to one or two such ring or to a benzene ring. In the case of a polycyclic carbocycle, the attachment point may be on any ring. Examples of carbocycles include, but are not limited to, cyclopropyl, cyclobutyl, cyclopentyl, cyclohexyl, cyclohexenyl, cycloheptyl, cycloheptenyl, bicyclo[3.3.0]octane, indane, bicyclo[3.3.1]nonane, decalin, tetrahydronaphthalene, spiro[3.3]heptane, and bicyclo[3.1.0]hexane.

“Cycloalkyl” refers to a saturated ring containing the specified number of ring carbon atoms, and no heteroatom. In a like manner the term “C₃₋₆ cycloalkyl” refers to a saturated ring having from 3 to 6 ring carbon atoms. Exemplary “cycloalkyl” groups useful in the present invention include, but are not limited to, cyclopropyl, cyclobutyl, cyclopentyl and cyclohexyl.

“Halogen” or “halo” refers to fluorine, chlorine, bromine, or iodine.

“Haloalkyl” refers to an alkyl group as defined above in which one and up to all hydrogen atoms are replaced by a halogen; halogen is as defined herein. Similarly “fluoroalkyl” refers to an alkyl group as defined above in which one and up to all hydrogen atoms are replaced by fluorine. Examples of such branched or straight chained haloalkyl groups useful in the present invention include, but are not limited to, methyl, ethyl, propyl, isopropyl, isobutyl and n-butyl substituted independently with one or more halos, e.g., fluoro, chloro, bromo and iodo. Examples of “haloalkyl” include, but are not limited to, fluoromethyl, difluoromethyl, trifluoromethyl, 1-fluoroethyl, 2-fluoroethyl, 2,2-difluoroethyl, 2,2,2-trifluoroethyl, and perfluoro-n-propyl.

“Hydroxyalkyl” refers to an alkyl group as defined above in which one hydrogen on each carbon atom may be replaced by a hydroxy group. Examples of “hydroxyalkyl” include, but are not limited to, hydroxymethyl, hydroxyethyl, isopropanol, propane-1,2-diol.

“Heterocycle” or “heterocyclyl” refers to a non-aromatic saturated monocyclic or multicyclic ring system having 3 to 10 ring atoms, preferably 5 to 10 ring atoms, in which one or more of the atoms in the ring system is an element other than carbon, for example nitrogen, oxygen or sulfur, alone or in combination. There are no adjacent oxygen and/or sulfur atoms present in the ring system. Where the heterocycle contains a ring nitrogen, the heterocyclyl can be connected to the rest of the molecule via a ring carbon or nitrogen atom. The nitrogen or sulfur atom of the heterocyclyl can be optionally oxidized to the corresponding N-oxide, S-oxide or S,S-dioxide. Examples of heterocyclyl rings include, but are not limited to, azetidinyl, piperidyl, pyrrolidinyl, piperazinyl, morpholinyl, thiomorpholinyl, thiazolidinyl, 1,4-dioxanyl, tetrahydrofuranyl, tetrahydrothiophenyl, decahydroquinolinyl, and the like.

“Heteroaryl” means an aromatic monocyclic or multicyclic ring system having 5 to 14 ring atoms, preferably 5 to 10 ring atoms, in which one or more of the ring atoms is an element other than carbon, for example nitrogen, oxygen or sulfur, alone or in combination. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, pyrazolo[1,5-a]pyrimidinyl, imidazo[1,2-a]pyridinyl, imidazo[2,1-b]thiazolyl, indazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, naphthyridinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula (I), and pharmaceutically acceptable excipients.

As used herein, the term “optionally” means that the subsequently described event(s) may or may not occur, and includes both event(s), which occur, and events that do not occur.

As used herein, the term “substituted with one or more groups” refers to substitution with the named substituent or substituents, multiple degrees of substitution, up to replacing all hydrogen atoms with the same or different substituents, being allowed unless the number of substituents is explicitly stated. Where the number of substituents is not explicitly stated, one or more is intended.

Each variable is independently defined each time it occurs within the generic structural formula definitions. For example, when there is more than one substituent on a “Z” ring, each substituent is independently selected at each occurrence, and each substituent can be the same or different from the other(s).

The term “Syk inhibitor”, is used to mean a compound which inhibits the Syk enzyme.

The term “Syk mediated disease” or a “disorder or disease or condition mediated by inappropriate Syk activity” is used to mean any disease state mediated or modulated by Syk kinase mechanisms. Such disease states may include inflammatory, allergic and autoimmune diseases, for example, asthma, chronic obstructive pulmonary disease (COPD), adult respiratory distress syndrome (ARDs), ulcerative colitis, Crohns disease, bronchitis, dermatitis, allergic rhinitis, psoriasis, scleroderma, urticaria, rheumatoid arthritis, multiple sclerosis, cancer, HIV and lupus, in particular, asthma, chronic obstructive pulmonary disease (COPD), adult respiratory distress syndrome (ARDs), allergic rhinitis and rheumatoid arthritis.

As used herein, “a compound of the invention” means a compound of Formula (I) or a salt, or solvate thereof.

As used herein, the term “solvate” refers to a complex of variable stoichiometry formed by a solute (in this invention, a compound of Formula (I), or a salt thereof) and a solvent. Such solvents for the purpose of the invention may not interfere with the biological activity of the solute. Examples of suitable solvents include, but are not limited to, water, acetone, methanol, ethanol and acetic acid. Preferably the solvent used is a pharmaceutically acceptable solvent. Examples of suitable pharmaceutically acceptable solvents include water, ethanol and acetic acid. Most preferably the solvent is water.

As used herein, the term “prodrug” refers to a compound (e.g., a drug precursor) that is transformed in vivo to yield a parent compound or a pharmaceutically acceptable salt, hydrate or solvate of the parent compound. The transformation may occur by various mechanisms (e.g., by metabolic or chemical processes), such as, for example, through hydrolysis in blood. Prodrugs are such derivatives, and a discussion of the use of prodrugs is provided by. “Prodrugs: Challenges and Rewards, Parts 1 and 2,” Vol. V of the Biotechnology: Pharmaceutical Aspects (Ronald T. Borchardt and C. Russel Middaugh, series editors), ed. Valentino J. Stella, Ronald T. Borchardt, Michael J. Hageman, Reza Oliyai, Hans Maag, Jefferson W. Tilley, American Association of Pharmaceutical Scientists and Springer, 2007.

The compounds of Formula (I) may have the ability to crystallize in more than one form, a characteristic known as polymorphism, and it is understood that such polymorphic forms (“polymorphs”) are within the scope of Formula (I). Polymorphism generally can occur as a response to changes in temperature or pressure or both and can also result from variations in the crystallization process. Polymorphs can be distinguished by various physical characteristics known in the art such as x-ray diffraction patterns, solubility and melting point.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by chromatography employing columns with a chiral stationary phase. Also, some of the compounds of Formula (I) may be atropisomers (e.g., substituted biaryls) and are considered as part of this invention.

It is also noted that the compounds of Formula (I) may form tautomers. It is understood that all tautomers and mixtures of tautomers of the compounds of the present invention are included within the scope of the compounds of the present invention. Any compounds described herein containing olefinic double bonds, unless specified otherwise, are meant to include both E and Z geometric isomers.

Whilst the embodiments for each variable have generally been listed above separately for each variable, this invention also includes those compounds in which several or each embodiment in Formula (I) is selected from each of the embodiments listed above. Therefore, this invention is intended to include all combinations of embodiments for each variable.

The compounds of the present invention may be in the form of and/or may be administered as a pharmaceutically acceptable salt. For a review on suitable salts see Berge et al., J. Pharm. Sci. 1977, 66, 1-19. Typically, the salts of the present invention are pharmaceutically acceptable salts. Salts encompassed within the term “pharmaceutically acceptable salts” refer to non-toxic salts of the compounds of this invention. Suitable pharmaceutically acceptable salts can include acid or base additions salts.

A pharmaceutically acceptable acid addition salt can be formed by reaction of a compound of Formula (I) with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated, for example, by crystallisation and filtration. A pharmaceutically acceptable acid addition salt of a compound of Formula (I) can comprise or be, for example, a hydrobromide, hydrochloride, sulfate, nitrate, phosphate, succinate, maleate, formarate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g., 2-naphthalenesulfonate) or hexanoate salt.

A pharmaceutically acceptable base salt can be formed by reaction of a compound of Formula (I) with a suitable inorganic or organic base. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Other, non-pharmaceutically acceptable, salts, e.g., oxalates or trifluoroacetates, may also be used, for example, in the isolation of compounds of the invention, and are included within the scope of this invention.

The invention includes within its scope all possible stoichiometric and non-stoichiometric forms of the compounds of Formula (I).

In the compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula (I). For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula (I) can be prepared without undue experimentation by conventional techniques well known to those skilled in the art or by processes analogous to those described in the Schemes and Examples herein using appropriate isotopically-enriched reagents and/or intermediates

### Compounds of the Invention

In some embodiments, the compounds of Formula (I) are prodrugs of trans-4-[(1R)-(6-{[4-(difluoromethyl)pyridin-2-yl]amino}-4-methyl-2,3′-bipyridin-6′-yl)-1-hydroxyethyl]cyclohexanecarboxylic acid, a potent inhibitor of Syk activity, and thus are potentially useful in the treatment of diseases and conditions associated with inappropriate Syk activity. In some embodiments, the compounds of Formula (I) and salts and solvates thereof, themselves, are believed to be inhibitors of Syk activity, and thus be potentially useful in the treatment of diseases and conditions associated with inappropriate Syk activity.

In embodiment no. 1, the invention provides a compound of the Formula (I)

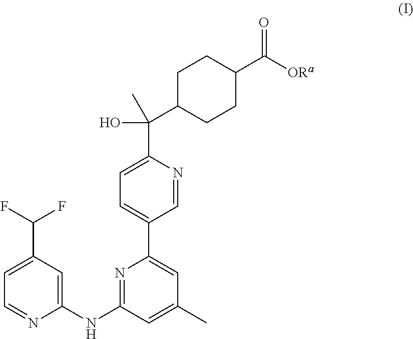

or a pharmaceutically acceptable salt thereof, wherein R^{a} is selected from the group consisting of:
A. C₁₋₃alkyl substituted by 1 to 3 moieties selected from the group consisting of —OH, —N(R^{e})₂, —N(C₁₋₄alkyl)₃, —C(O)N(R^{e})₂, —S—(C₁₋₄ alkyl), —S(O)—(C₁₋₄ alkyl), and —S(O)₂—(C₁₋₄ alkyl);
B. C₄₋₈alkyl, wherein said C₄₋₈alkyl is unsubstituted or substituted by 1-3 moieties selected from the group consisting of —OH, —N(R^{e})₂, —N(C₁₋₄alkyl)₃, —C(O)N(R^{e})₂, —S—(C₁₋₄ alkyl), —S(O)—(C₁₋₄ alkyl), and —S(O)₂—(C₁₋₄ alkyl);
C. a group of the formula -M-R^{CH}, wherein

1. M is a bond or —(CH₂)₁₋₂—;

2. R^{CH} is a cyclic moiety selected from the group consisting of:
a.) aryl or carbocyclyl;
b.) a 5- to 9-membered mono or bicyclic heterocyclyl containing 1 or 2 heteroatoms independently selected from the group consisting of N and O; and
c.) 5- to 6-membered heteroaryl containing 1 or 4 heteroatoms independently selected from the group consisting of N, O, and S;
wherein R^{CH} is unsubstituted or substituted with 1-4 moieties independently selected from the group consisting of halo, C₁₋₄alkyl, C₁₋₄alkoxy, —CN, —CO₂H, —OH, —N(R^{e})₂, and —N(C₁₋₄ alkyl)₃;
D. a group of the formula

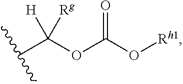

wherein

1. R^{g} is H or C₁₋₄alkyl; and

2. R^{h1} is
a.) —Y¹—C^{A}; wherein
   (i) Y¹ is a bond, —CH₂—, or —CH₂CH₂—O—CH₂—; and
   (ii) C^{A} is a cyclic moiety selected from the group consisting of:
      (I) C₃₋₆ cycloalkyl;
      (II) phenyl;
      (III) a 5- or 6-membered heterocyclyl containing 1 to 2 heteroatoms selected from the group consisting of N and O; and
      (IV) a 5- or 6-membered heteroaryl containing 1 to 2 heteroatoms selected from the group consisting of N and 0;
   wherein C^{A} is unsubstituted or substituted by 1 to 3 moieties independently selected from the group consisting of C₁₋₃alkyl, halo, or —N(R^{e})₂;
b.) C₂₋₆ alkyl, wherein said C₂₋₆alkyl of R^{h1} is unsubstituted or substituted by 1 to 3 moieties independently selected from the group consisting of —OH, C₁₋₃alkoxy, —CH₂—O—C₁₋₃alkyl, —N(R^{e})₂, —S—C₁₋₃alkyl, —S(O)—C₁₋₃alkyl, —S(O)₂—C₁₋₃alkyl, —P(O)(OH)₂, and —C(O)—N(R^{e})₂;
c.) a group of the formula

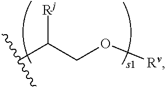

wherein
(i) R^{j} is H C₁₋₃alkyl, or —CH₂—O—C₁₋₃alkyl;
(ii) R^{v} is H, C₁₋₃alkyl, or —P(O)(OH)₂, and
(iii) the subscript s1 is 2, 3, 4, or 5;
E. a group of the formula

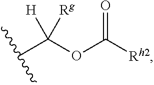

wherein

1. R^{g} is as set forth above;

2. R^{h2} is:
a.) —Y²—C^{B}; wherein
   (i) Y² is a bond or a group

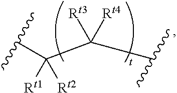

wherein
(I) R^{t1} and R^{t2} are independently H, C₁₋₃alkyl or —OH;
(II) each occurrence of R^{t3} and R^{t4} are independently H or C₁₋₃alkyl; and
(III) the subscript t is 0, 1, 2, 3, or 4;
(ii) C^{B} is a cyclic moiety selected from the group consisting of:
   (I) C₄₋₆cycloalkyl, cholic acid, or chenodeoxycholic acid,
   (II) phenyl,
   (III) 5- or 6-membered heterocyclyl containing 1 to 2 heteroatoms selected from the group consisting of N, O, and S; and
   (IV) 5- or 6-membered heteroaryl containing 1 to 2 heteroatoms selected from the group consisting of N, O, and S; and
   wherein C^{B} is unsubstituted or substituted by 1 to 3 moieties independently selected from the group consisting of C₁₋₃alkyl, C₁₋₃alkoxy, —OH, oxo, and C₁₋₃acyloxy;
b.) C₂₋₆ alkyl, wherein said C₂₋₆alkyl of R^{h2} is unsubstituted or substituted by 1 to 3 moieties independently selected from the group consisting of —OH, —N(R^{e})₂, —N(H)C(O)—C₁₋₃alkyl, and —N(H)C(O)—CH₂-phenyl;
c.) a group of the formula

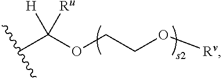

wherein:
(i) R^{u} is H or C₁₋₃alkyl;
(ii) the subscript s2 is 0, 1, 2, 3, 4, or 5; and
(iii) R^{v} is as set forth above;
F. a group of the formula

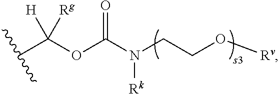

wherein

1. R^{k} is H or C₁₋₃alkyl;

2. the subscript s3 is 1, 2, 3, 4, or 5;

3. R^{g} and R^{v} are as set forth above;
G. a group of the formula

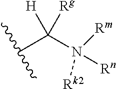

1. R^{g} is as set forth above;
2. R^{k2} is present or absent, and if present is H, C₁₋₈alkyl, —(CH₂)ₓ—CN, or C₃₋₆cycloalkyl (the dashed line indicating that the substituent R^{k2} is present or absent, and if present, the illustrated N atom bears a positive charge);
3. R^{m} and Rⁿ are independently:
   a) C₁₋₈alkyl,
   b) —(CH₂)ₓ—CN,
   c) —(CH₂)ₓ—N(R^{e})₂,
   d) —(CH₂)₂O—(CH₂)₂N(R^{e})₂,
   e) —(CH₂)ₓ—C(O)₂—C₁₋₃alkyl,
   f) —Y³—C^{C}, wherein:
      (i) Y³ is a bond, C₁₋₄alkylene, or —(CH₂)₂O—C(O)—; and
      (ii) C^{C} is a cyclic moiety selected from the group consisting of C₃₋₆cycloalkyl, phenyl, or pyridyl; or
   g) R^{m} and Rⁿ together with the N atom to which they are attached form a 5- to 6-membered heterocyclyl or heteroaryl containing 1 to 4 heteroatoms selected from the group consisting of N and O, wherein said heterocyclyl or heteroaryl is unsubstituted or is substituted by 1 to 3 moieties selected from the group consisting of C₁₋₃alkyl, —C(O)—C₁₋₃alkyl, —C(O)—N(R^{e})₂, fluoro, —C₁₋₃alkyl-OH, and —OH.
H. a group of the formula

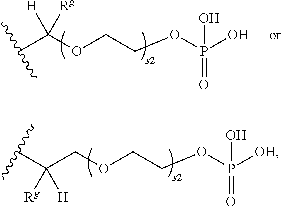

wherein R^{g} and the subscript s2 are as set forth above;

each R^{e} is independently H or C₁₋₄alkyl; and
each occurrence of the subscript x is independently 1, 2, or 3; and
wherein the compound is other than one of the following compounds or a pharmaceutically acceptable salt thereof:
methyl 4-[(6-{[4-(difluoromethyl)pyridin-2-yl]amino}-4-methyl-2,3′-bipyridin-6′-yl)-1-hydroxyethyl]cyclohexanecarboxylate;
ethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-hydroxyethyl 4-[1-(6-{[4-(difluoromethyl)pyridin-2-yl]amino}-4-methyl-2,3′-bipyridin-6′-yl)-1-hydroxyethyl]cyclohexanecarboxylate;
benzyl 4-[1-(6-{[4-(difluoromethyl)pyridin-2-yl]amino}-4-methyl-2,3′-bipyridin-6′-yl)-1-hydroxyethyl]cyclohexanecarboxylate;
propyl 4-[1-(6-{[4-(difluoromethyl)pyridin-2-yl]amino}-4-methyl-2,3′-bipyridin-6′-yl)-1-hydroxyethyl]cyclohexanecarboxylate;
propan-2-yl 4-[1-(6-{[4-(difluoromethyl)pyridin-2-yl]amino}-4-methyl-2,3′-bipyridin-6′-yl)-1-hydroxyethyl]cyclohexanecarboxylate;
butyl 4-[1-(6-{[4-(difluoromethyl)pyridin-2-yl]amino}-4-methyl-2,3′-bipyridin-6′-yl)-1-hydroxyethyl]cyclohexanecarboxylate;
butan-2-yl 4-[1-(6-{[4-(difluoromethyl)pyridin-2-yl]amino}-4-methyl-2,3′-bipyridin-6′-yl)-1-hydroxyethyl]cyclohexanecarboxylate;
2-methylpropyl 4-[1-(6-{[4-(difluoromethyl)pyridin-2-yl]amino}-4-methyl-2,3′-bipyridin-6′-yl)-1-hydroxyethyl]cyclohexanecarboxylate;
pentyl 4-[1-(6-{[4-(difluoromethyl)pyridin-2-yl]amino}-4-methyl-2,3′-bipyridin-6′-yl)-1-hydroxyethyl]cyclohexanecarboxylate;
2,2-dimethylpropyl 4-[1-(6-{[4-(difluoromethyl)pyridin-2-yl]amino}-4-methyl-2,3′-bipyridin-6′-yl)-1-hydroxyethyl]cyclohexanecarboxylate;
hexyl 4-[−1-(6-{[4-(difluoromethyl)pyridin-2-yl]amino}-4-methyl-2,3′-bipyridin-6′-yl)-1-hydroxyethyl]cyclohexanecarboxylate;
heptyl 4-[1-(6-{[4-(difluoromethyl)pyridin-2-yl]amino}-4-methyl-2,3′-bipyridin-6′-yl)-1-hydroxyethyl]cyclohexanecarboxylate;
octyl 4-[1-(6-{[4-(difluoromethyl)pyridin-2-yl]amino}-4-methyl-2,3′-bipyridin-6′-yl)-1-hydroxyethyl]cyclohexanecarboxylate;
cyclohexyl 4-[1-(6-{[4-(difluoromethyl)pyridin-2-yl]amino}-4-methyl-2,3′-bipyridin-6′-yl)-1-hydroxyethyl]cyclohexanecarboxylate;
tetrahydro-2H-pyran-4-yl 4-[1-(6-{[4-(difluoromethyl)pyridin-2-yl]amino}-4-methyl-2,3′-bipyridin-6′-yl)-1-hydroxyethyl]cyclohexanecarboxylate;
cyclohexylmethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-methoxyethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-(2-ethoxyethoxyl)ethyl 4-[−1-(6-{[4-(difluoromethyl)pyridin-2-yl]amino}-4-methyl-2,3′-bipyridin-6′-yl)-1-hydroxyethyl]cyclohexanecarboxylate;
2-methoxy-2-oxoethyl 4-[−1-(6-{[4-(difluoromethyl)pyridin-2-yl]amino}-4-methyl-2,3′-bipyridin-6′-yl)-1-hydroxyethyl]cyclohexanecarboxylate;
2-(dimethylamino)-2-oxoethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-(morpholin-4-yl)ethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-(dimethylamino)ethyl 4-[1-(6-{[4-(difluoromethyl)pyridin-2-yl]amino}-4-methyl-2,3′-bipyridin-6′-yl)-1-hydroxyethyl]cyclohexanecarboxylate;
(5-methyl-2-oxo-1,3-dioxol-4-yl)methyl 4-[1-(6-{[4-(difluoromethyl)pyridin-2-yl]amino}-4-methyl-2,3′-bipyridin-6′-yl)-1-hydroxyethyl]cyclohexanecarboxylate;
[(ethoxycarbonyl)oxy]methyl 4-[1-(6-{[4-(difluoromethyl)pyridin-2-yl]amino}-4-methyl-2,3′-bipyridin-6′-yl)-1-hydroxyethyl]cyclohexanecarboxylate;
{[(propan-2-yloxy)carbonyl]oxy}methyl 4-[1-(6-{[4-(difluoromethyl)pyridin-2-yl]amino}-4-methyl-2,3′-bipyridin-6′-yl)-1-hydroxyethyl]cyclohexanecarboxylate;
1-[(ethoxycarbonyl)oxy]ethyl 4-[1-(6-{[4-(difluoromethyl)pyridin-2-yl]amino}-4-methyl-2,3′-bipyridin-6′-yl)-1-hydroxyethyl]cyclohexanecarboxylate;
1-{[(propan-2-yloxy)carbonyl]oxy}ethyl 4-[1-(6-{[4-(difluoromethyl)pyridin-2-yl]amino}-4-methyl-2,3′-bipyridin-6′-yl)-1-hydroxyethyl]cyclohexanecarboxylate;
1-{[(cyclohexyloxy)carbonyl]oxy}ethyl 4-[1-(6-{[4-(difluoromethyl)pyridin-2-yl]amino}-4-methyl-2,3′-bipyridin-6′-yl)-1-hydroxyethyl]cyclohexanecarboxylate;
(acetyloxy)methyl 4-[1-(6-{[4-(difluoromethyl)pyridin-2-yl]amino}-4-methyl-2,3′-bipyridin-6′-yl)-1-hydroxyethyl]cyclohexanecarboxylate;
[(2,2-dimethylpropanoyl)oxy]methyl 4-[1-(6-{[4-(difluoromethyl)pyridin-2-yl]amino}-4-methyl-2,3′-bipyridin-6′-yl)-1-hydroxyethyl]cyclohexanecarboxylate;
1-(acetyloxy)ethyl 4-[1-(6-{[4-(difluoromethyl)pyridin-2-yl]amino}-4-methyl-2,3′-bipyridin-6′-yl)-1-hydroxyethyl]cyclohexanecarboxylate;
1-[(2-methylpropanoyl)oxy]ethyl 4-[1-(6-{[4-(difluoromethyl)pyridin-2-yl]amino}-4-methyl-2,3′-bipyridin-6′-yl)-1-hydroxyethyl]cyclohexanecarboxylate; and
2-(acetyloxy)ethyl 4-[1-(6-{[4-(difluoromethyl)pyridin-2-yl]amino}-4-methyl-2,3′-bipyridin-6′-yl)-1-hydroxyethyl]cyclohexanecarboxylate.

In embodiment no. 2, the invention provides a compound of the Formula (I), wherein R^{a} is C₁₋₃alkyl substituted by 1 to 3 moieties selected from the group consisting of —OH, —N(R^{e})₂, —N(C₁₋₄alkyl)₃, —C(O)N(R^{e})₂, —S—(C₁₋₄ alkyl), —S(O)—(C₁₋₄ alkyl), and —S(O)₂—(C₁₋₄ alkyl).

In embodiment no. 3, the invention provides a compound of the Formula (I), wherein R^{a} is C₄₋₈alkyl, wherein said C₄₋₈alkyl is unsubstituted or substituted by 1-3 moieties selected from the group consisting of —OH, —N(R^{e})₂, —N(C₁₋₄alkyl)₃, —C(O)N(R^{e})₂, —S—(C₁₋₄ alkyl), —S(O)—(C₁₋₄ alkyl), and —S(O)₂—(C₁₋₄ alkyl).

In embodiment no. 4, the invention provides a compound of the Formula (I), wherein R^{a} is the group of the formula -M-R^{CH}.

In embodiment no. 5, the invention provides a compound of the Formula (I), wherein R^{a} is as set forth in embodiment no. 4,

M is a bond or —CH₂—;

R^{CH} is phenyl, cyclobutyl, cyclohexyl, tetrahydropyranyl, dioxolanyl, piperidinyl, or imidazolyl, wherein R^{CH} is unsubstituted or substituted as set forth in embodiment no. 1.

In embodiment no. 6, the invention provides a compound of the Formula (I), wherein R^{a} is the group of the formula

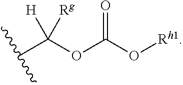

In embodiment no. 7, the invention provides a compound of the Formula (I), wherein R^{a} is a set forth in embodiment no. 6;

R^{g} is —H, —CH₃, or —C(H)(CH)₂;

R^{h1} is a group of the formula

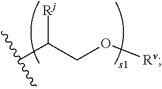

and

R^{j}, R^{v} and the subscript s1 are as set forth in embodiment no. 1.

In embodiment no. 8, the invention provides a compound of the Formula (I), wherein R^{a} is the group of the formula

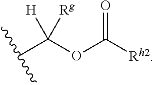

In embodiment no. 9, the invention provides a compound of the Formula (I), wherein R^{a} is as set forth in embodiment no. 8 and R^{g} is H.

In embodiment no. 10, the invention provides a compound of the Formula (I), wherein R^{a} is as set forth in embodiment no. 8; R^{g} is H; and

R^{h2} is a group of the formula

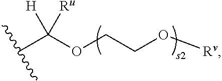

wherein R^{u}, R^{v}, and the subscript s2 are as set forth in embodiment no. 1

In embodiment no. 11, the invention provides a compound of the Formula (I), wherein R^{a} is the group of the formula

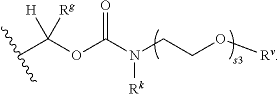

In embodiment no. 12, the invention provides a compound of the Formula (I), wherein R^{a} is as set forth in embodiment no. 11;

R^{g} is H,

R^{k} is —H or —CH₃,

the subscript s3 is 1 or 2; and

R^{v} is —CH₃.

In embodiment no. 13, the invention provides a compound of the Formula (I), wherein R^{a} is the group of the formula

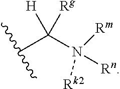

The dashed line in the illustrated group indicates that the substituent R^{k2} is present or absent, and if present, the illustrated N atom bears a positive charge.

In embodiment no. 14, the invention provides a compound of the Formula (I), wherein R^{a} is as set forth in embodiment no. 13 and R^{g} is H.

In embodiment no. 15, the invention provides a compound of the Formula (I), wherein R^{a} is as set forth in embodiment no. 13,

R^{g} is H, and

R^{k}, R^{m}, and Rⁿ are C₁₋₆alkyl.

In embodiment no. 16, the invention provides a compound of the Formula (I), wherein R^{a} is as set forth in embodiment no. 8,

R^{g} is H,

R^{m} and Rⁿ together with the N atom to which they are attached form a piperidinyl or piperazinyl ring wherein said piperidinyl or piperazinyl is unsubstituted or substituted by 1 moeity selected from the group consistent of by 1 to 3 moieties selected from the group consisting of C₁₋₃alkyl, —C(O)—C₁₋₃alkyl, —C(O)—N(R^{e})₂, fluoro, —C₁₋₃alkyl-OH, and —OH;

R^{k} is —CH₃, —CH₂CH₃, or —CH₂CN; and

R^{e} is as set forth in embodiment no. 1.

In embodiment no. 17, the invention provides a compound of the Formula (I), wherein R^{a} is as set forth in embodiment no. 13,

R^{g} is H,

R^{m} and Rⁿ together with the N atom to which they are attached form a 5-membered heteroaryl ring containing from 1 to 4 N atoms, wherein said heteroaryl ring is unsubstituted or substituted by one C₁₋₃ alkyl.

In embodiment no. 18, the invention provides a compound of the Formula (I), wherein R^{a} is the group of the formula

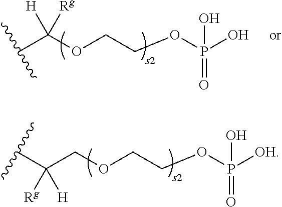

In embodiment no. 19, the invention provides a compound of the Formula (I),
2-hydroxy-2-methylpropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
cis-4-aminocyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(2S)-2,3-dihydroxypropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
6-O-({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)-D-glucopyranose;
trans-3-aminocyclobutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
phenyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-(methylsulfanyl)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-(methylsulfonyl)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
3-(methylsulfanyl)propyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]-N,N,N-trimethylethanaminium;
(1-methyl-1H-imidazol-2-yl)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-hydroxy-3-methylbutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-(dimethylamino)propyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(4R)-2,2-dimethyl-1,3-dioxolan-4-yl]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(4S)-2,2-dimethyl-1,3-dioxolan-4-yl]methyl trans 4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(2R)-2,3-dihydroxypropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
piperidin-4-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
8-azabicyclo[3.2.1]oct-3-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
trans-3-aminocyclobutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
tetrahydro-2H-pyran-4-ylmethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
piperidin-4-ylmethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-(methylamino)propyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
trans-4-aminocyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
trans-4-(methylamino)cyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
6-(dimethylamino)hexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
methyl 6-O-({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)-alpha-D-glucopyranoside;
3-(dimethylamino)-3-oxopropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
4-methoxyphenyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
4-cyanophenyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[(6-aminohexyl)oxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-oxo-2,4,7,10-tetraoxadodec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[2-(benzyloxy)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
15-hydroxy-3-oxo-2,4,7,10,13-pentaoxapentadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-oxo-2,4,7,10,13-pentaoxapentadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2-ethoxyethoxy)carbonyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({[6-(dimethylamino)hexyl]oxy}carbonyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[2-ethoxy-1-(ethoxymethyl)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(tetrahydro-2H-pyran-4-ylmethoxy)carbonyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
({[2-(methylsulfanyl)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[(trans-4-aminocyclohexyl)oxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[3-(dimethylamino)-3-oxopropoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[(1-methyl-1H-imidazol-2-yl)methoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
1-methyl-3-oxo-2,4,7,10-tetraoxadodec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
1-methyl-3-oxo-2,4,7,10-tetraoxadodec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
1-(1-methylethyl)-3-oxo-2,4,7,10-tetraoxadodec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl piperidine-4-carboxylate;
1-[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]ethyl 1-methyl-1H-pyrazole-4-carboxylate;
[(2-hydroxy-2-methylpropanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(3-hydroxy-3-methylbutanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(hydroxyacetyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2S)-2-hydroxy-3-(4-hydroxyphenyl)propanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl N-acetyl-L-leucinate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 1-methyl-1H-pyrazole-4-carboxylate;
[(3-hydroxypropanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl (3 alpha,7 alpha, 12alpha)-3,7,12-trihydroxycholan-24-oate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl (3 alpha,7alpha)-3,7-dihydroxycholan-24-oate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl benzoate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl pyridine-3-carboxylate;
{[3-(3-hydroxyphenyl)propanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 5-oxo-D-prolinate;
{[(2S)-2-hydroxy-3-methylbutanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2R)-2-hydroxypropanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[3-(3,4-dihydroxyphenyl)propanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2R)-2-hydroxy-2-phenylacetyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 4-hydroxybenzoate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 2-(acetyloxy)benzoate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 2-hydroxybenzoate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 2,5-dihydroxybenzoate;
[(3-hydroxybutanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(1-hydroxycyclobutyl)carbonyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2S)-2-hydroxy-4-methylpentanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(N-acetylseryl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl N˜2˜N˜6˜-bis[(benzyloxy)carbonyl]-L-lysinate;
1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2-methoxyethyl)(methyl)carbamoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-oxo-2,7,10-trioxa-4-azaundec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
4-{[({trans-4-[(1R)-1-(6-{[4-(Difluoromethyl)pyridin-2-yl]amino}-4-methyl-2,3′-bipyridin-6′-yl)-1-hydroxyethyl]cyclohexyl}carbonyl)oxy]methyl}-4-methylmorpholin-4-ium iodide;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dimethylpropan-2-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N-ethyl-N-(1-methylethyl)propan-2-aminium;
N,N-dibutyl-N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}butan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-2-(dimethylamino)-N,N-dimethylethanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-diethylbutan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-diethyl-4-hydroxypentan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dioctyloctan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dipropylpropan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-diethylethanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dimethylcyclohexanaminium;
4-(diethylcarbamoyl)-1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-1-methylpiperazin-1-ium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-4,4-difluoro-1-methylpiperidinium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-1-ethylpiperidinium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dimethyl-1-phenylethanaminium;
1-(cyanomethyl)-1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}piperidinium;
4-acetyl-1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-1-methylpiperidinium;
N-butyl-N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N-methylbutan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-2-ethoxy-N,N-dimethyl-2-oxoethanaminium;
N-benzyl[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]-N,N-dimethylmethanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dimethyl-2-pyridin-2-ylethanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-diethylcyclohexanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-2-[2-(dimethylamino)ethoxy]-N,N-dimethylethanaminium;
N-cyclohexyl-N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N-methylcyclohexanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dimethyl-2-[(phenylcarbonyl)oxy]ethanaminium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-1,4-dimethylpiperazin-1-ium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-4-hydroxy-1-methylpiperidinium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-4-(2-hydroxyethyl)-1-methylpiperazin-1-ium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-4-(hydroxymethyl)-1-methylpiperidinium;
1-{[({trans-4-[(1R)-1-(6-{[4-(Difluoromethyl)pyridin-2-yl]amino}-4-methyl-2,3′-bipyridin-6′-yl)-1-hydroxyethyl]cyclohexyl}carbonyl)oxy]methyl}-3-methyl-1H-imidazol-3-ium;
(5-methyl-1H-tetrazol-1-yl)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(5-methyl-2H-tetrazol-2-yl)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
1H-imidazol-1-ylmethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-[(R)-methylsulfinyl]ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-[(S)-methylsulfinyl]ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[3-(methylsulfonyl)propoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-(methylsulfinyl)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
3-(methylsulfinyl)propyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[2-(methylsulfinyl)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[2-(methylsulfinyl)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[2-(methylsulfonyl)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
17,17-dihydroxy-17-oxido-3-oxo-2,4,7,10,13,16-hexaoxa-17λ⁵-phosphaheptadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-(phosphonooxy)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
trans-4-(dimethylamino)cyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate; and
(phosphonooxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate or a pharmaceutically acceptable salt thereof.

### Uses of the Compounds

While not being bound by any specific theory, in some embodiments of the invention, the compound of Formula (I) function as prodrugs, and cleave under physiological conditions to release the compound, trans-4-[(1R)-(6-{[4-(difluoromethyl)pyridin-2-yl]amino}-4-methyl-2,3′-bipyridin-6′-yl)-1-hydroxyethyl]cyclohexanecarboxylic acid. In some embodiments, the compound of Formula (I) inhibit Syk by themselves.

Compound of Formula (I) or its pharmaceutically acceptable salts and pharmaceutical compositions can be used to treat or prevent a variety of conditions or diseases mediated by Spleen tyrosine kinase (Syk). Such conditions and diseases include, but are not limited to: (1) arthritis, including rheumatoid arthritis, juvenile arthritis, psoriatic arthritis and osteoarthritis; (2) asthma and other obstructive airways diseases, including chronic asthma, late asthma, airway hyper-responsiveness, bronchitis, bronchial asthma, allergic asthma, intrinsic asthma, extrinsic asthma, dust asthma, adult respiratory distress syndrome, recurrent airway obstruction, and chronic obstruction pulmonary disease including emphysema; (3) autoimmune diseases or disorders, including those designated as single organ or single cell-type autoimmune disorders, for example Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia including idiopathic thrombopenic purpura, sympathetic ophthalmia, myasthenia gravis, Graves' disease, primary biliary cirrhosis, chronic aggressive hepatitis, ulcerative colitis and membranous glomerulopathy, those designated as involving systemic autoimmune disorder, for example systemic lupus erythematosis, immune thrombocytopenic purpura, rheumatoid arthritis, Sjogren's syndrome, Reiter's syndrome, polymyositis-dermatomyositis, systemic sclerosis, polyarteritis nodosa, multiple sclerosis and bullous pemphigoid, and additional autoimmune diseases, which can be B-cell (humoral) based or T-cell based, including Cogan's syndrome, ankylosing spondylitis, Wegener's granulomatosis, autoimmune alopecia, Type I or juvenile onset diabetes, and thyroiditis; (4) cancers or tumors, including alimentary/gastrointestinal tract cancer, colon cancer, liver cancer, skin cancer including mast cell tumor and squamous cell carcinoma, breast and mammary cancer, ovarian cancer, prostate cancer, lymphoma and leukemia (including but not limited to acute myelogenous leukemia, chronic myelogenous leukemia, mantle cell lymphoma, NHL B cell lymphomas (e.g., precursor B-ALL, marginal zone B cell lymphoma, chronic lymphocytic leukemia, diffuse large B cell lymphoma, Burkitt lymphoma, mediastinal large B-cell lymphoma), Hodgkin lymphoma, NK and T cell lymphomas; TEL-Syk and ITK-Syk fusion driven tumors) myelomas including multiple myeloma, myeloproliferative disorders kidney cancer, lung cancer, muscle cancer, bone cancer, bladder cancer, brain cancer, melanoma including oral and metastatic melanoma, Kaposi's sarcoma, proliferative diabetic retinopathy, and angiogenic-associated disorders including solid tumors, and pancreatic cancer; (5) diabetes, including Type I diabetes and complications from diabetes; (6) eye diseases, disorders or conditions including autoimmune diseases of the eye, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, and ocular neovascularization; (7) intestinal inflammations, allergies or conditions including Crohn's disease and/or ulcerative colitis, inflammatory bowel disease, coeliac diseases, proctitis, eosinophilic gastroenteritis, and mastocytosis; (8) neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial ischemica, renal ischemia, heart attacks, cardiac hypertrophy, atherosclerosis and arteriosclerosis, organ hypoxia; (9) platelet aggregation and diseases associated with or caused by platelet activation, such as arteriosclerosis, thrombosis, intimal hyperplasia and restenosis following vascular injury; (10) conditions associated with cardiovascular diseases, including restenosis, acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, and the like; (11) skin diseases, conditions or disorders including atopic dermatitis, eczema, psoriasis, scleroderma, pruritus and other pruritic conditions; (12) allergic reactions including anaphylaxis, allergic rhinitis, allergic dermatitis, allergic urticaria, angioedema, allergic asthma, or allergic reaction to insect bites, food, drugs, or pollen; (13) transplant rejection, including pancreas islet transplant rejection, bone marrow transplant rejection, graft-versus-host disease, organ and cell transplant rejection such as bone marrow, cartilage, cornea, heart, intervertebral disc, islet, kidney, limb, liver, lung, muscle, myoblast, nerve, pancreas, skin, small intestine, or trachea, and xeno transplantation; (14) low grade scarring including scleroderma, increased fibrosis, keloids, post-surgical scars, pulmonary fibrosis, cystic fibrosis, vascular spasms, migraine, reperfusion injury, and post-myocardial infarction.

The invention thus provides compounds of Formula (I) and salts, solvates and physiologically functional derivatives thereof for use in therapy, and particularly in the treatment of diseases and conditions mediated by inappropriate Syk activity. The inappropriate Syk activity referred to herein is any Syk activity that deviates from the normal Syk activity expected in a particular mammalian subject. Inappropriate Syk activity may take the form of, for instance, an abnormal increase in activity, or an aberration in the timing and or control of Syk activity. Such inappropriate activity may result then, for example, from overexpression or mutation of the protein kinase leading to inappropriate or uncontrolled activation.

In a further embodiment, the present invention is directed to methods of regulating, modulating, or inhibiting Syk for the prevention and/or treatment of disorders related to unregulated Syk activity.

In a further embodiment, the present invention provides a method of treatment of a mammal suffering from a disorder mediated by Syk activity, which comprises administering to said mammal an effective amount of a compound of Formula (I) or a pharmaceutically acceptable salt or solvate thereof.

In a further embodiment, the present invention provides for the use of a compound of Formula (I), or a pharmaceutically acceptable salt or solvate thereof, in the preparation of a medicament for the treatment of a disorder mediated by Syk activity.

In a further embodiment said disorder mediated by Syk activity is asthma. In a further embodiment said disorder is rheumatoid arthritis. In yet another embodiment, said disorder is cancer. In a further embodiment said disorder is ocular conjunctivitis.

Yet another aspect of the present invention provides a method for treating diseases caused by or associated with Fc receptor signaling cascades, including FceRI and/or FcgRI-mediated degranulation as a therapeutic approach towards the treatment or prevention of diseases characterized by, caused by and/or associated with the release or synthesis of chemical mediators of such Fc receptor signaling cascades or degranulation. In addition, Syk is known to play a critical role in immunotyrosine-based activation motif (ITAM) signaling, B cell receptor signaling, T cell receptor signaling and is an essential component of integrin beta (1), beta (2), and beta (3) signaling in neutrophils. Thus, compounds of the present invention can be used to regulate Fc receptor, ITAM, B cell receptor and integrin signaling cascades, as well as the cellular responses elicited through these signaling cascades. Non-limiting examples of cellular responses that may be regulated or inhibited include respiratory burst, cellular adhesion, cellular degranulation, cell spreading, cell migration, phagocytosis, calcium ion flux, platelet aggregation and cell maturation.

### Compositions and Administration

While it is possible that, for use in therapy, a compound of Formula (I), as well as salts, solvates and physiological functional derivatives thereof, may be administered as the raw chemical, it is possible to present the active ingredient as a pharmaceutical composition. Accordingly, the invention further provides a pharmaceutical composition, which comprises a compound of Formula (I) and salts, solvates and physiological functional derivatives thereof, and one or more pharmaceutically acceptable carriers, diluents, or excipients. The compounds of the Formula (I) and salts and thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical composition including admixing a compound of the Formula (I), or salts, solvates and physiological functional derivatives thereof, with one or more pharmaceutically acceptable carriers, diluents or excipients.

In some embodiments, the compounds of Formula (I) provide advantages in formulating aqueous solutions containing such compounds. Due to the improved aqueous solubilities of the compounds of Formula (I) in comparison with the parent compound, trans-4-[(1R)-(6-{[4-(difluoromethyl)pyridin-2-yl]amino}-4-methyl-2,3′-bipyridin-6′-yl)-1-hydroxyethyl]cyclohexanecarboxylic acid, formulations containing therapeutically useful concentrations of compounds. Accordingly, aqueous parenteral formulations containing suitable concentrations of the compounds of the Formula (I) can be prepared. The aqueous solubilities of the compounds of Formula (I) in biorelevant can be determined as described in the working examples below.

Pharmaceutical compositions of the present invention may be presented in unit dose forms containing a predetermined amount of active ingredient per unit dose. Such a unit may contain, for example, 5 μg to 1 g, preferably 1 mg to 700 mg, more preferably 5 mg to 100 mg of a compound of the Formula (I), depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions of the present invention may be adapted for administration by any appropriate route, for example by the oral (including buccal or sublingual), rectal, topical, inhaled, nasal, ocular, or parenteral (including intravenous and intramuscular) route. Such compositions may be prepared by any method known in the art of pharmacy, for example by bringing into association the active ingredient with the carrier(s) or excipient(s). Dosage forms include tablets, troches, dispersions, suspensions, solutions, capsules, creams, ointments, aerosols, and the like.

In a further embodiment, the present invention provides a pharmaceutical composition adapted for administration by the oral route, for treating, for example, rheumatoid arthritis.

In a further embodiment, the present invention provides a pharmaceutical composition adapted for administration by the nasal route, for treating, for example, allergic rhinitis.

In a further embodiment, the present invention provides a pharmaceutical composition adapted for administration by the inhaled route, for treating, for example, asthma, COPD or ARDS.

In a further embodiment, the present invention provides a pharmaceutical composition adapted for administration by the ocular route, for treating, diseases of the eye, for example, conjunctivitis.

In a further embodiment, the present invention provides a pharmaceutical composition adapted for administration by the parenteral (including intravenous) route, for treating, for example, cancer.

Pharmaceutical compositions of the present invention which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release, for example, by coating or embedding particulate material in polymers, wax or the like.

The compounds of Formula (I), and salts and solvates thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of Formula (I) and salts and solvates thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Dosage forms for inhaled administration may conveniently be formulated as aerosols or dry powders.

For compositions suitable and/or adapted for inhaled administration, it is preferred that the compound or salt of Formula (I) is in a particle-size-reduced form, and more preferably the size-reduced form is obtained or obtainable by micronisation. The preferable particle size of the size-reduced (e.g., micronised) compound or salt or solvate is defined by a D50 value of about 0.5 to about 10 microns (for example as measured using laser diffraction).

Aerosol formulations, e.g., for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g., co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, it is preferred that the pharmaceutical composition is a dry powder inhalable composition. Such a composition can comprise a powder base such as lactose, glucose, trehalose, mannitol or starch, the compound of Formula (I) or salt or solvate thereof (preferably in particle-size-reduced form, e.g., in micronised form), and optionally a performance modifier such as L-leucine or another amino acid, and/or metals salts of stearic acid such as magnesium or calcium stearate. Preferably, the dry powder inhalable composition comprises a dry powder blend of lactose and the compound of Formula (I) or salt thereof. The lactose is preferably lactose hydrate e.g., lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g., 10-1000 microns e.g., 30-1000 microns) in diameter, and/or 50% or more of the lactose particles being less than 500 microns (e.g., 10-500 microns) in diameter. More preferably, the particle size of the lactose is defined by 90% or more of the lactose particles being less than 300 microns (e.g., 10-300 microns e.g., 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. It is preferable that about 3 to about 30% (e.g., about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 J D Zwolle, Netherlands).

Optionally, in particular for dry powder inhalable compositions, a pharmaceutical composition for inhaled administration can be incorporated into a plurality of sealed dose containers (e.g., containing the dry powder composition) mounted longitudinally in a strip or ribbon inside a suitable inhalation device. The container is rupturable or peel-openable on demand and the dose of e.g., the dry powder composition can be administered by inhalation via the device such as the DISKUS® device (GlaxoSmithKline). Other dry powder inhalers are well known to those of ordinary skill in the art, and many such devices are commercially available, with representative devices including Aerolizer® (Novartis), Airmax™ (WAX), ClickHaler® (Innovata Biomed), Diskhaler® (GlaxoSmithKline), Accuhaler (GlaxoSmithKline), Easyhaler® (Orion Pharma), Eclipse™ (Aventis), FlowCaps® (Hovione), Handihaler® (Boehringer Ingelheim), Pulvinal® (Chiesi), Rotahaler® (GlaxoSmithKline), SkyeHaler™ or Certihaler™(SkyePharma), Twisthaler® (Schering-Plough), Turbuhaler® (AstraZeneca), Ultrahaler® (Aventis), and the like.

Dosage forms for ocular administration may be formulated as solutions or suspensions with excipients suitable for ophthalmic use.

Dosage forms for nasal administration may conveniently be formulated as aerosols, solutions, drops, gels or dry powders.

Pharmaceutical compositions adapted for administration by inhalation include fine particle dusts or mists, which may be generated by means of various types of metered, dose pressurised aerosols, nebulizers or insufflators.

For pharmaceutical compositions suitable and/or adapted for intranasal administration, the compound of Formula (I) or a pharmaceutically acceptable salt or solvate thereof may be formulated as a fluid formulation for delivery from a fluid dispenser. Such fluid dispensers may have, for example, a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO-A-2005/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. A particularly preferred fluid dispenser is of the general type illustrated in FIGS. 30-40 of WO-A-2005/044354.

The following are examples of representative pharmaceutical dosage forms for the compounds of this invention:

| Injectable Suspension (I.M.) | mg/ml |
|---|---|
| Compound of Formula (I) | 10 |
| Methylcellulose | 5.0 |
| Tween 80 | 0.5 |
| Benzyl alcohol | 9.0 |
| Benzalkonium chloride | 1.0 |
| Water for injection to a total volume of 1 ml | |

| Tablet | mg/tablet |
|---|---|
| Compound of Formula (I) | 25 |
| Microcrystalline Cellulose | 415 |
| Providone | 14.0 |
| Pregelatinized Starch | 43.5 |
| Magnesium Stearate | 2.5 |
| | 500 |

| Capsule | mg/capsule |
|---|---|
| Compound of Formula (I) | 25 |
| Lactose Powder | 573.5 |
| Magnesium Stearate | 1.5 |
| | 600 |

| Inhalation Aerosol | Per dose | |
|---|---|---|
| Compound of Formula (I) | 100 | mcg |
| Oleic Acid | 5 | mcg |
| Ethanol | 1 | mg |
| HFA 227 (1,1,1,2,3,3,3-heptafluoropropane) | 75 | mg |

| Dry Powder Inhalation Aerosol | Per dose | |
|---|---|---|
| Compound of Formula (I) | 100 | mcg |
| Lactose | 12.5 | mg |

It will be appreciated that when the compound of the present invention is administered in combination with other therapeutic agents normally administered by the inhaled, intravenous, oral or intranasal route, that the resultant pharmaceutical composition may be administered by the same routes.

It should be understood that in addition to the ingredients particularly mentioned above, the compositions may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of Formula (I) for the treatment of diseases or conditions associated with inappropriate Syk activity, will generally be in the range of 5 g to 100 mg/kg body weight of recipient (mammal) per day and more usually in the range of 5 g to 10 mg/kg body weight per day. This amount may be given in a single dose per day or more usually in a number (such as two, three, four, five or six) of sub-doses per day such that the total daily dose is the same. An effective amount of a salt or solvate, thereof, may be determined as a proportion of the effective amount of the compound of Formula (I) per se.

### Combination Therapy

Compounds of the present invention, and their salts and solvates thereof, may be employed alone or in combination with other therapeutic agents for the treatment of diseases and conditions associated with inappropriate Syk activity. Combination therapies according to the present invention thus comprise the administration of at least one compound of Formula (I) or a pharmaceutically acceptable salt or solvate thereof, and the use of at least one other pharmaceutically active agent. The compound(s) of Formula (I) and the other pharmaceutically active agent(s) may be administered together or separately and, when administered separately this may occur simultaneously or sequentially in any order. The amounts of the compound(s) of Formula (I) and the other pharmaceutically active agent(s) and the relative timings of administration will be selected in order to achieve the desired combined therapeutic effect.

For the treatment of the inflammatory diseases, rheumatoid arthritis, psoriasis, inflammatory bowel disease, COPD, asthma and allergic rhinitis a compound of Formula (I) may be combined with one or more other active agents such as: (1) TNF-α inhibitors such as infliximab (Remicade®), etanercept (Enbrel®), adalimumab (Humira®), certolizumab pegol (Cimzia®), and golimumab (Simponi®); (2) non-selective COX-I/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, etodolac, azapropazone, pyrazolones such as phenylbutazone, salicylates such as aspirin); (3) COX-2 inhibitors (such as meloxicam, celecoxib, rofecoxib, valdecoxib and etoricoxib); (4) other agents for treatment of rheumatoid arthritis including methotrexate, leflunomide, sulfasalazine, azathioprine, cyclosporin, tacrolimus, penicillamine, bucillamine, actarit, mizoribine, lobenzarit, ciclesonide, hydroxychloroquine, d-penicillamine, aurothiomalate, auranofin or parenteral or oral gold, cyclophosphamide, Lymphostat-B, BAFF/APRIL inhibitors and CTLA-4-Ig or mimetics thereof; (5) leukotriene biosynthesis inhibitor, 5-lipoxygenase (5-LO) inhibitor or 5-lipoxygenase activating protein (FLAP) antagonist such as zileuton; (6) LTD4 receptor antagonist such as zafirlukast, montelukast and pranlukast; (7) PDE4 inhibitor such as roflumilast, cilomilast, AWD-12-281 (Elbion), and PD-168787 (Pfizer); (8) antihistaminic H1 receptor antagonists such as cetirizine, levocetirizine, loratadine, desloratadine, fexofenadine, astemizole, azelastine, levocabastine, olopatidine, methapyrilene and chlorpheniramine; (9) α1- and α2-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride; (10) anticholinergic agents such as ipratropium bromide, tiotropium bromide, oxitropium bromide, aclidinium bromide, glycopyrrolate, (R,R)-glycopyrrolate, pirenzepine, and telenzepine; (11) β-adrenoceptor agonists such as metaproterenol, isoproterenol, isoprenaline, albuterol, formoterol (particularly the fumarate salt), salmeterol (particularly the xinafoate salt), terbutaline, orciprenaline, bitolterol mesylate, fenoterol, and pirbuterol, or methylxanthanines including theophylline and aminophylline, sodium cromoglycate; (12) insulin-like growth factor type I (IGF-1) mimetic; (13) glucocorticosteroids, especially inhaled glucocorticoid with reduced systemic side effects, such as prednisone, prednisolone, flunisolide, triamcinolone acetonide, beclomethasone dipropionate, budesonide, fluticasone propionate, ciclesonide and mometasone furoate; (14) kinase inhibitors such as inhibitors of the Janus Kinases (JAK 1 and/or JAK2 and/or JAK 3 and/or TYK2), p38 MAPK and IKK2; (15) B-cell targeting biologics such as rituximab (Rituxan®); (16) selective costimulation modulators such as abatacept (Orencia); (17) interleukin inhibitors, such as IL-1 inhibitor anakinra (Kineret) and IL-6 inhibitor tocilizumab (Actemra).

The present invention also provides for so-called “triple combination” therapy, comprising a compound of Formula (I) or a pharmaceutically acceptable salt thereof together with beta2-adrenoreceptor agonist and an anti-inflammatory corticosteroid. Preferably this combination is for treatment and/or prophylaxis of asthma, COPD or allergic rhinitis. The beta2-adrenoreceptor agonist and/or the anti-inflammatory corticosteroid can be as described above and/or as described in WO 03/030939 A1. Representative examples of such a “triple” combination are a compound of Formula (I) or a pharmaceutically acceptable salt thereof in combination with the components of Advair® (salmeterol xinafoate and fluticasone propionate), Symbicort® (budesonide and formoterol fumarate), or Dulera® (mometasone furoate and formoterol fumarate) or a pharmaceutically acceptable salt thereof (e.g., salmeterol xinafoate and fluticasone propionate).

For the treatment of cancer a compound of Formula (I) may be combined with one or more of an anticancer agent. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: (1) estrogen receptor modulator such as diethylstibestral, tamoxifen, raloxifene, idoxifene, LY353381, LY117081, toremifene, fluoxymestero, and SH646; (2) other hormonal agents including aromatase inhibitors (e.g., aminoglutethimide, tetrazole anastrozole, letrozole and exemestane), luteinizing hormone release hormone (LHRH) analogues, ketoconazole, goserelin acetate, leuprolide, megestrol acetate and mifepristone; (3) androgen receptor modulator such as finasteride and other 5α-reductase inhibitors, nilutamide, flutamide, bicalutamide, liarozole, and abiraterone acetate; (4) retinoid receptor modulator such as bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide; (5) antiproliferative agent such as antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, aminopterin, 5-flurouracil, floxuridine, methotrexate, leucovarin, hydroxyurea, thioguanine (6-TG), mercaptopurine (6-MP), cytarabine, pentostatin, fludarabine phosphate, cladribine (2-CDA), asparaginase, gemcitabine, alanosine, swainsonine, lometrexol, dexrazoxane, methioninase, and 3-aminopyridine-2-carboxaldehyde thiosemicarbazone; (6) prenyl-protein transferase inhibitor including farnesyl-protein transferase (FPTase), geranylgeranyl-protein transferase type I (GGPTase-I), and geranylgeranyl-protein transferase type-II (GGPTase-II, also called Rab GGPTase); (7) HMG-CoA reductase inhibitor such as lovastatin, simvastatin, pravastatin, atorvastatin, fluvastatin and rosuvastatin; (8) angiogenesis inhibitor such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, erythropoietin (epoietin-α), granulocyte-CSF (filgrastin), granulocyte, macrophage-CSF (sargramostim), pentosan polysulfate, cyclooxygenase inhibitors, steroidal anti-inflammatories, carboxyamidotriazole, combretastatin A-4, squalamine, 6-O-chloroacetyl-carbonyl)-fumagillol, thalidomide, angiostatin, troponin-1, angiotensin II antagonists, heparin, carboxypeptidase U inhibitors, and antibodies to VEGF, endostatin, ukrain, ranpimase, IM862, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, and 3-[(2,4-dimethylpyrrol-5-yl)methylene]-2-indolinone (SU5416); (9) PPAR-γ agonists, PPAR-δ agonists, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and (2R)-7-(3-(2-chloro-4-(4-fluorophenoxyl)phenoxy)propoxy)-2-ethylchromane-2-carboxylic acid (disclosed in U.S. Ser. No. 60/235,708 and 60/244,697); (9) inhibitor of inherent multidrug resistance including inhibitors of p-glycoprotein (P-gp), such as LY335979, XR9576, OC144-093, R101922, VX853 and PSC833 (valspodar); (10) inhibitor of cell proliferation and survival signaling such as inhibitors of EGFR (for example gefitinib and erlotinib), inhibitors of ERB-2 (for example trastuzumab), inhibitors of IGF 1R such as MK-0646 (dalotuzumab), inhibitors of CD20 (rituximab), inhibitors of cytokine receptors, inhibitors of MET, inhibitors of PI3K family kinase (for example LY294002), serine/threonine kinases (including but not limited to inhibitors of Akt such as described in (WO 03/086404, WO 03/086403, WO 03/086394, WO 03/086279, WO 02/083675, WO 02/083139, WO 02/083140 and WO 02/083138), inhibitors of Raf kinase (for example BAY-43-9006), inhibitors of MEK (for example CI-1040 and PD-098059) and inhibitors of mTOR (for example Wyeth CCI-779 and Ariad AP23573); (11) a bisphosphonate such as etidronate, pamidronate, alendronate, risedronate, zoledronate, ibandronate, incadronate or cimadronate, clodronate, EB-1053, minodronate, neridronate, piridronate and tiludronate; (12) γ-secretase inhibitors, (13) agents that interfere with receptor tyrosine kinases (RTKs) including inhibitors of c-Kit, Eph, PDGF, Flt3 and c-Met; (14) agent that interferes with a cell cycle checkpoint including inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032; (15) BTK inhibitors such as PCI32765, AVL-292 and AVL-101; (16) PARP inhibitors including iniparib, olaparib, AGO14699, ABT888 and MK4827; (16) ERK inhibitors; (17) mTOR inhibitors such as sirolimus, ridaforolimus, temsirolimus, everolimus; (18) cytotoxic/cytostatic agents.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, chlorambucil, cyclophosphamide, ifosfamide, mechlorethamine, melphalan, uracil mustard, thiotepa, busulfan, carmustine, lomustine, streptozocin, tasonermin, lonidamine, carboplatin, altretamine, dacarbazine, procarbazine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, doxorubicin, daunorubicin, idarubicin, anthracenedione, bleomycin, mitomycin C, dactinomycin, plicatomycin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl-4-methylsulphonyl-daunorubicin.

An example of a hypoxia activatable compound is tirapazamine.

Examples of proteasome inhibitors include but are not limited to lactacystin and bortezomib.

Examples of microtubule inhibitors/microtubule-stabilising agents include vincristine, vinblastine, vindesine, vinzolidine, vinorelbine, vindesine sulfate, 3′,4′-didehydro-4′-deoxy-8′-norvincaleukoblastine, podophyllotoxins (e.g., etoposide (VP-16) and teniposide (VM-26)), paclitaxel, docetaxol, rhizoxin, dolastatin, mivobulin isethionate, auristatin, cemadotin, RPR109881, BMS184476, vinflunine, cryptophycin, anhydrovinblastine, N,N-dimethyl-L-valyl-L-valyl-N-methyl-L-valyl-L-prolyl-L-proline-t-butylamide, TDX258, the epothilones (see, for example, U.S. Pat. Nos. 6,284,781 and 6,288,237) and BMS188797).

Some examples of topoisomerase inhibitors are topotecan, hycaptamine, irinotecan, rubitecan, 6-ethoxypropionyl-3′,4′-O-exo-benzylidene-chartreusin, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, 2,3-(methylenedioxy)-5-methyl-7-hydroxy-8-methoxybenzo[c]-phenanthridinium, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins include, but are not limited to inhibitors of KSP, inhibitors of MKLP1, inhibitors of CENP-E, inhibitors of MCAK, inhibitors of Kif14, inhibitors of Mphosph1 and inhibitors of Rab6-KIFL.

Examples of “histone deacetylase inhibitors” include, but are not limited to, vorinostat, trichostatin A, oxamflatin, PXD101, MG98, valproic acid and scriptaid.

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N6-[4-deoxy-4-[N2-[2,4-tetradecadienoyl]glycylamino]-L-glycero-B-L-manno-heptopyranosyl]adenine, aplidine, ecteinascidin, troxacitabine, aminopterin, 5-flurouracil, floxuridine, methotrexate, leucovarin, hydroxyurea, thioguanine (6-TG), mercaptopurine (6-MP), cytarabine, pentostatin, fludarabine phosphate, cladribine (2-CDA), asparaginase, gemcitabine, alanosine, swainsonine, lometrexol, dexrazoxane, methioninase, and 3-aminopyridine-2-carboxaldehyde thiosemicarbazone.

Non-limiting examples of suitable agents used in cancer therapy that may be combined with compounds of Formula (I) include, but are not limited to, abarelix; aldesleukin; alemtuzumab; alitretinoin; allopurinol; altretamine; amifostine; anastrozole; arsenic trioxide; asparaginase; azacitidine; bendamustine; bevacuzimab; bexarotene; bleomycin; bortezomib; busulfan; calusterone; capecitabine; carboplatin; carmustine; cetuximab; chlorambucil; cisplatin; cladribine; clofarabine; cyclophosphamide; cytarabine; dacarbazine; dactinomycin, actinomycin D; dalteparin; darbepoetin alfa; dasatinib; daunorubicin; degarelix; denileukin diftitox; dexrazoxane; docetaxel; doxorubicin; dromostanolone propionate; eculizumab; Elliott's B Solution; eltrombopag; epirubicin; epoetin alfa; erlotinib; estramustine; etoposide phosphate; etoposide; everolimus; exemestane; filgrastim; floxuridine; fludarabine; fluorouracil; fulvestrant; gefitinib; gemcitabine; gemtuzumab ozogamicin; goserelin acetate; histrelin acetate; hydroxyurea; ibritumomab tiuxetan; idarubicin; ifosfamide; imatinib mesylate; interferon alfa 2a; interferon alfa-2b; irinotecan; ixabepilone; lapatinib; lenalidomide; letrozole; leucovorin; leuprolide acetate; levamisole; lomustine; meclorethamine, nitrogen mustard; megestrol acetate; melphalan, L-PAM; mercaptopurine; mesna; methotrexate; methoxsalen; mitomycin C; mitotane; mitoxantrone; nandrolone phenpropionate; nelarabine; nilotinib; Nofetumomab; ofatumumab; oprelvekin; oxaliplatin; paclitaxel; palifermin; pamidronat; panitumumab; pazopanib; pegademase; pegaspargase; Pegfilgrastim; pemetrexed disodium; pentostatin; pipobroman; plerixafor; plicamycin, mithramycin); porfimer sodium; pralatrexate; procarbazine; quinacrine; Rasburicase; raloxifene hydrochloride; Rituximab; romidepsin; romiplostim; sargramostim; sargramostim; satraplatin; sorafenib; streptozocin; sunitinib maleate; tamoxifen; temozolomide; temsirolimus; teniposide; testolactone; thioguanine; thiotepa; topotecan; toremifene; tositumomab; trastuzumab; tretinoin; uracil mustard; valrubicin; vinblastine; vincristine; vinorelbine; vorinostat; and zoledronate.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable diluent or carrier represent a further aspect of the invention. These combinations are of particular interest in respiratory diseases and are conveniently adapted for inhaled or intranasal delivery.

The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

### Methods of Preparing the Compounds of Formula (I)

The compounds of this invention may be made by a variety of methods, including standard chemistry. Illustrative general synthetic methods are set out below in the Schemes, and then specific compounds of the invention are prepared in the Examples. Any previously defined variable will continue to have the previously defined meaning unless otherwise indicated.

Compounds of Formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of protecting groups as well as the reaction conditions and order of reaction steps shall be consistent with the preparation of compounds of Formula (I). Those skilled in the art will recognize if a stereocenter exists in compounds of Formula (I). Accordingly, the present invention includes all possible stereoisomers and includes not only mixtures of stereoisomers (such as racemic compounds) but the individual stereoisomers as well. When a compound is desired as a single enantiomer, it may be obtained by stereospecific or stereoselective synthesis or by resolution of the final product or any convenient intermediate. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994).

The following abbreviations are used in the schemes and examples: Ac=Acetyl; AcOH=Acetic acid; Bn=benzyl; Boc (t-Boc)=t-butyloxycarbonyl; BOP=(Benzotriazol-1-yloxy)-tris(dimethylamino)phosphonium hexafluorophosphate; DAST=(Diethylamino)sulfur trifluoride; dba=dibenzylideneacetone; DCE=1,2-dichloroethane; DCM=Dichloromethane; Dibal/Dibal-H=Diisobutylaluminum hydride; DIPEA/DIEA=Diisopropylethylamine; DMAP=N,N-dimethyl-aminopyridine; DME=1,2-dimethoxyethane; DMF=Dimethyl formamide; DMSO=Dimethyl-sulfoxide; Dppf=1,1′-Bis(diphenylphosphino)ferrocene; EDC=N-(3-Dimethylaminopropyl)-N′-ethylcarbodiimide; EtOAc=Ethyl acetate; HATU=N,N,N′,N′-Tetramethyl-O-(7-azabenzotriazol-1-yl)uronium hexafluorophosphate; HMDS=Hexamethyldisilazane; HOBT=1-Hydroxybenzo-triazole; HPLC=high pressure liquid chromatography; IPA=Isopropyl alcohol; LDA=Lithium diisopropylamide; mCPBA=Meta-chloroperoxybenzoic acid; Ms=Methanesulfonyl (mesyl); MTBE=Methyl t-butyl ether; NBS=N-bromosuccinimide; Ph=phenyl; PyBrOP=Bromo-tris-pyrrolidino phosphoniumhexafluorophosphate; SFC=supercritical fluid chromatography; TBAF=t-butylammonium fluoride; TBDMS/TBS=t-butyl dimethylsilyl; TFA=Trifluoroacetic/trifluroacetate; THF=Tetrahydrofuran; TLC=Thin-layer chromatography; TMS=Trimethylsilyl; Ts=Toluenesulfonyl (tosyl); TSA=p-toluenesulfonic acid. Abbreviations for alkyl/cycloalkyl groups: Me=methyl, Et=ethyl, nPr=n-propyl, iPr=isopropyl, nBu=n-butyl, t-Bu=tertiary butyl, cPr=cyclopropyl, cBu=cyclobutyl, cPen=cyclopentyl, cHex=cyclohexyl, cHept=cycloheptyl.

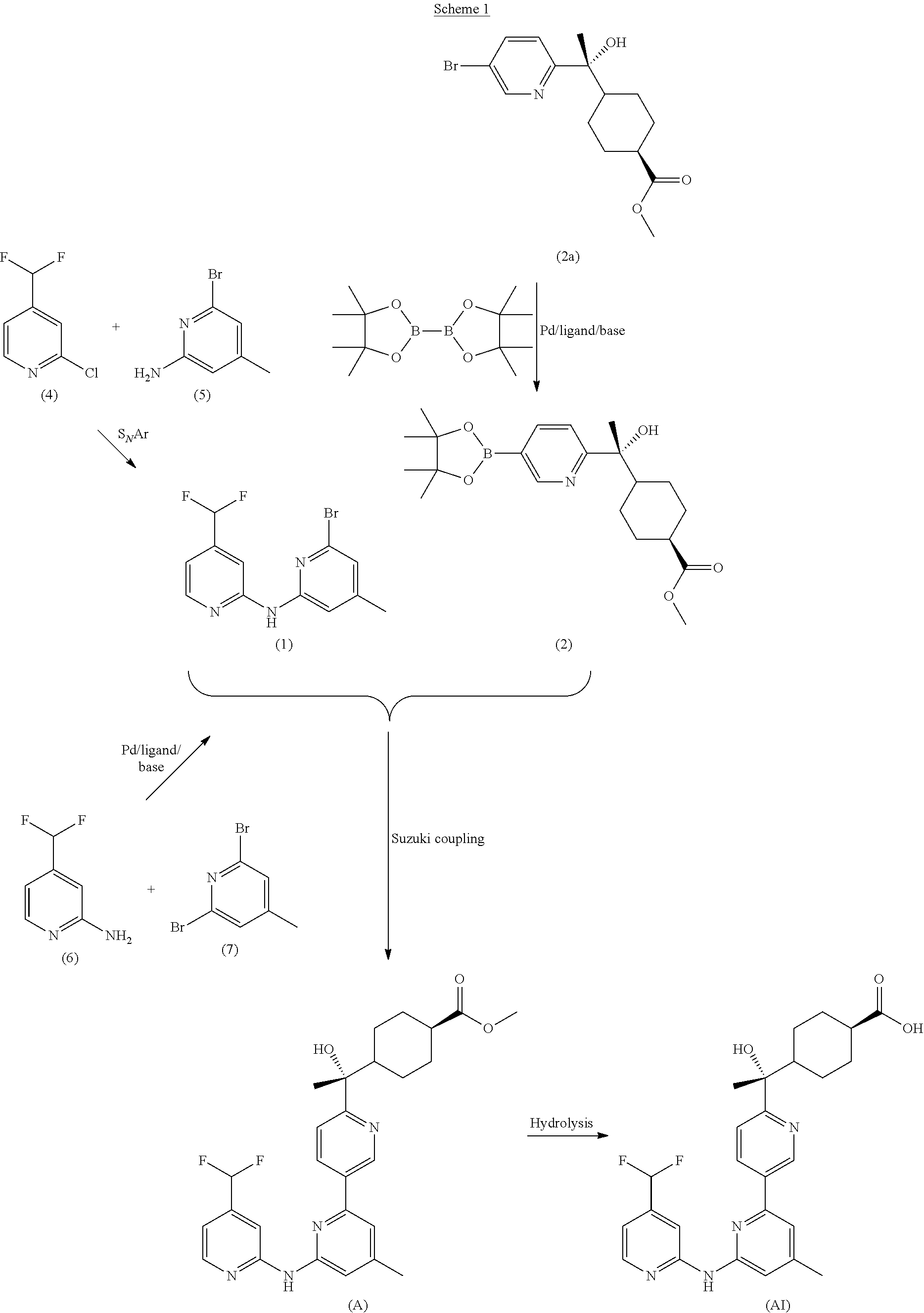

Intermediate (AI) is prepared through hydrolysis of the ester Intermediate (A), which is prepared by Suzuki coupling of pyridyl bromide (1) with pyridyl boronate esters (2). Pyridyl boronate esters (2) are formed by Miyaura borylation of the corresponding bromides (2a). Pyridyl bromides (1) are obtained by reacting 2-chloropyridines (4) and 2-amino-6-bromopyridines under base-mediated S_{N}Ar conditions. Alternatively, pyridyl bromides (1) can be formed by reaction of 2-aminopyridines with 2,6-dibromopyridines under palladium-mediated C—N coupling conditions.

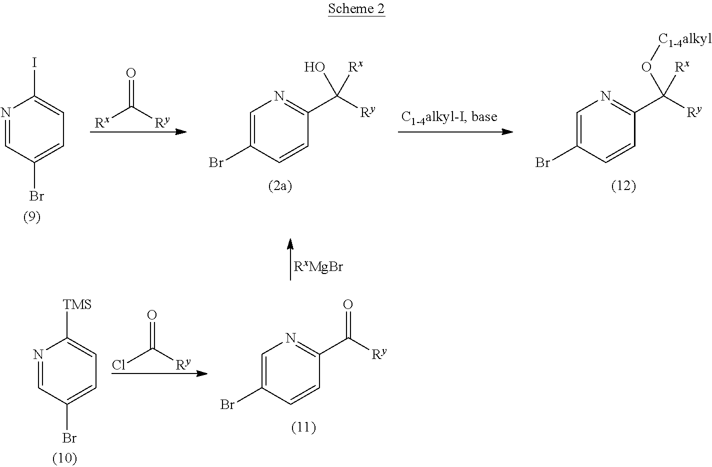

Compounds of formula (2a) are prepared by metallation of 2-iodopyridine (9) and addition to a carbonyl electrophile. Alternatively, 2-trimethylsilyl pyridine (10) reacts with acyl chlorides to afford pyridyl ketones (11), which react with Grignard reagents to form compounds of formula (8). Deprotonation of alcohol (8) and treatment with an electrophile gives compounds of formula (12). Compounds of formula (8) and (12) are converted to pyridyl boronates (2) and ultimately, compounds of Formula (I).

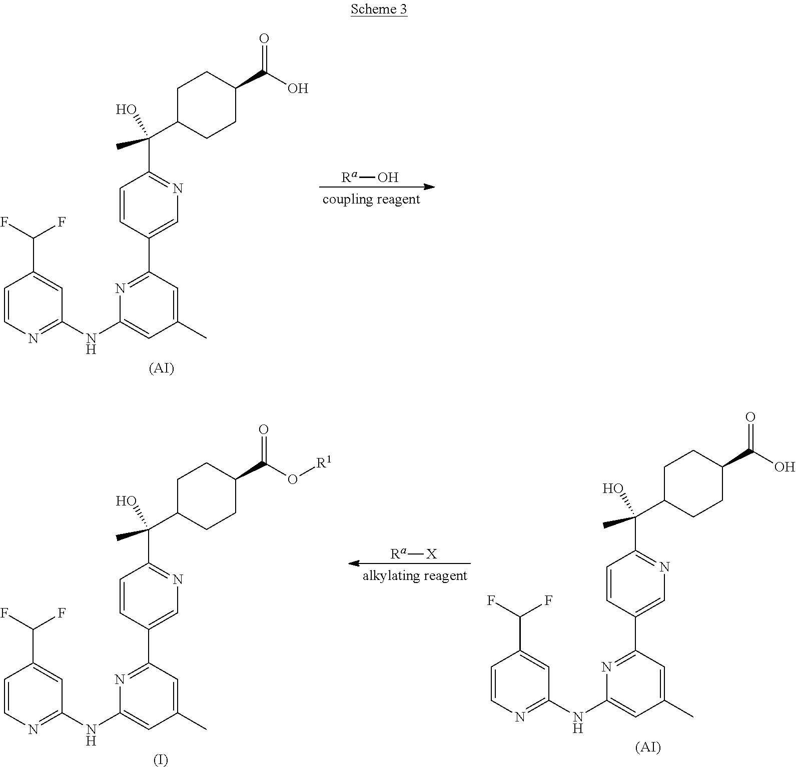

As shown in Scheme 3, certain compounds of Formula (I) are prepared from the trans-cyclohexane carboxylic acid (AI) by reactions with either alcohols (R^{a}—OH) or alkyl halides (R^{a}—X) under the appropriate conditions. In some preparations, compounds of Formula (I) are prepared by the coupling of the alcohol and carboxylic acid (AI) using standard coupling reaction conditions (Mitsunobu conditions, phosphonium-mediated couplings, carbodiimide-mediated couplings). In other preparations, compounds of Formula (I) are made through the coupling of alkyl halides and carboxylic acid (AI) under basic conditions.

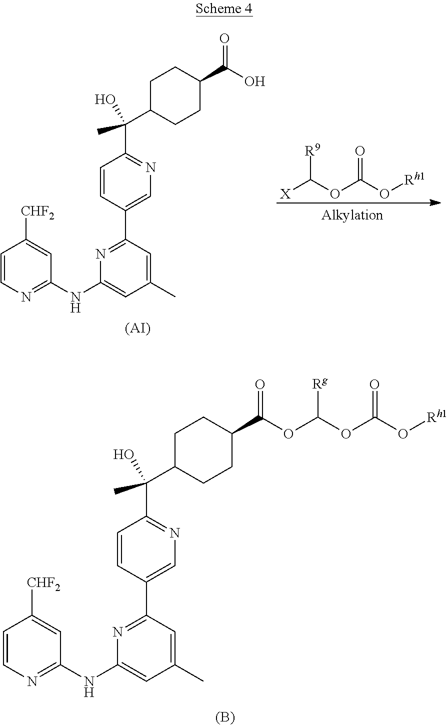

As shown in Scheme 4, ester carbonate acetals of structural subtype B are prepared from the trans-cyclohexane carboxylic acid (AI) and the appropriate haloalkyl carbonate in an alkylation reaction under basic conditions.

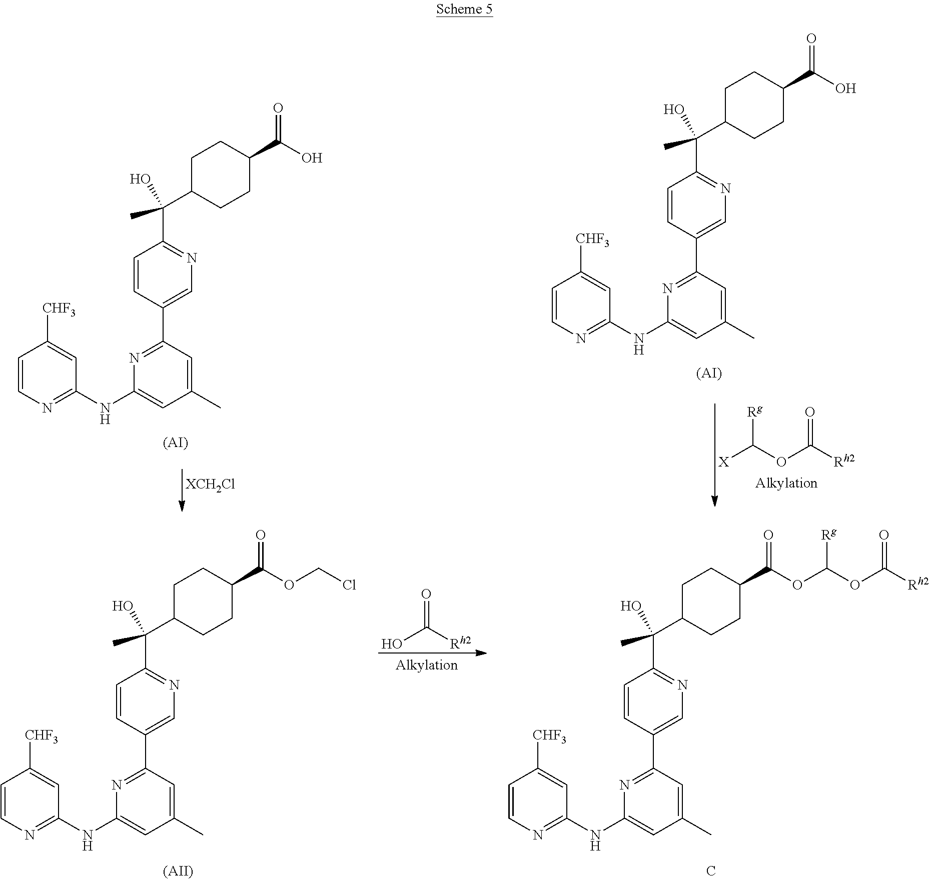

As shown in Scheme 5, diester acetals of structural subtype C are prepared from the trans-cyclohexane carboxylic acid (AI) either through the intermediacy of chloromethyl ester (AII) or direct alkylation of carboxylic acid (AI) with a haloalkylester reagent. The chloromethyl ester (AII) is prepared from a di-substituted methane derivative, such as bromochloromethane, and the carboxylic acid, and ester (AII) is then reacted with a carboxylic acid in an alkylation reaction under basic conditions. Alternatively, the carboxylic acid (AI) is treated with a haloalkylester under basic conditions to give diester acetals of type C.

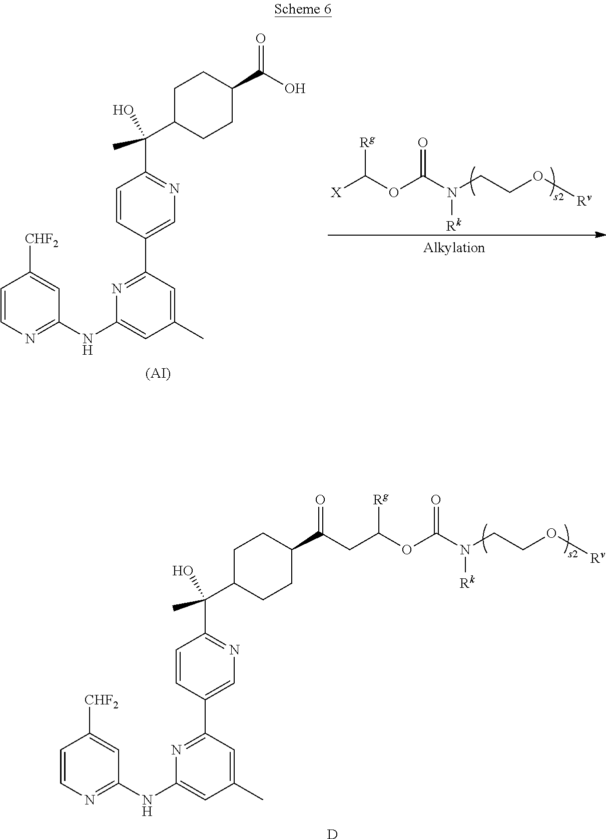

As shown in Scheme 6, ester carbamate acetals of structural subtype D are prepared from the trans-cyclohexane carboxylic acid (AI) and the appropriate haloalkyl carbamate in an alkylation reaction under basic conditions.

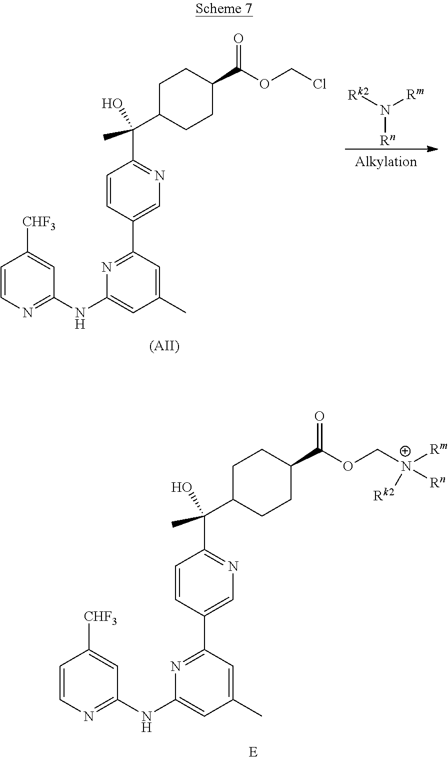

As shown in Scheme 7, quaternary amines of structural subtype E are prepared from the trans-cyclohexane chloromethyl ester (AII) and the appropriate tertiary amine in an alkylation reaction.

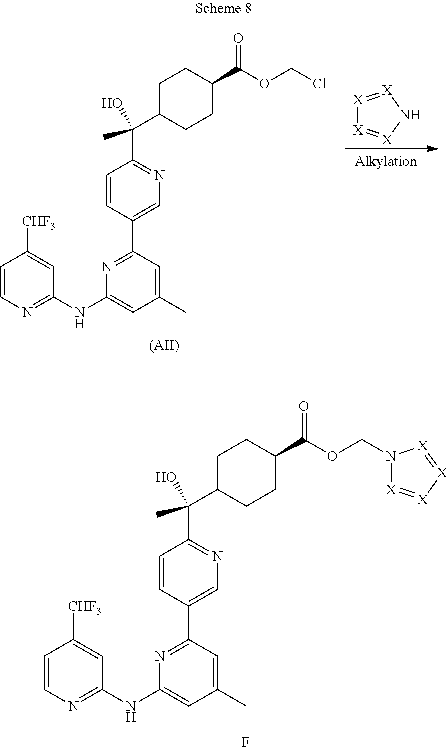

As shown in Scheme 8, ester heteroaryl acetals of structural subtype F (wherein the X in the illustrated ring represents a C or heteroatom) are prepared from the trans-cyclohexane carboxylic acid ester (AII) and the appropriate heteroaryl in an alkylation reaction.

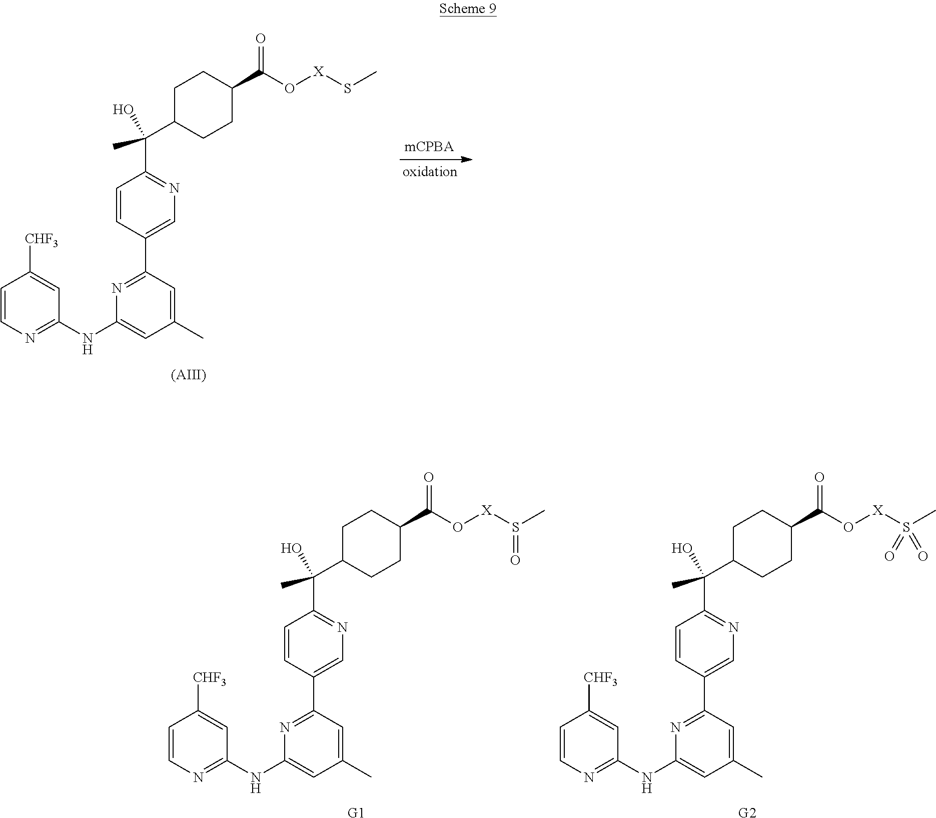

As shown in Scheme 9, sulfone and sulfoxide containing compounds of structural subtype GI and G2 (wherein X is a linking group), respectively, are prepared from the trans-cyclohexane carboxylic acid derivatives (AIII) that contain a sulfide through an oxidation reaction, employing typical oxidants like meta-chloroperoxybenzoic acid.

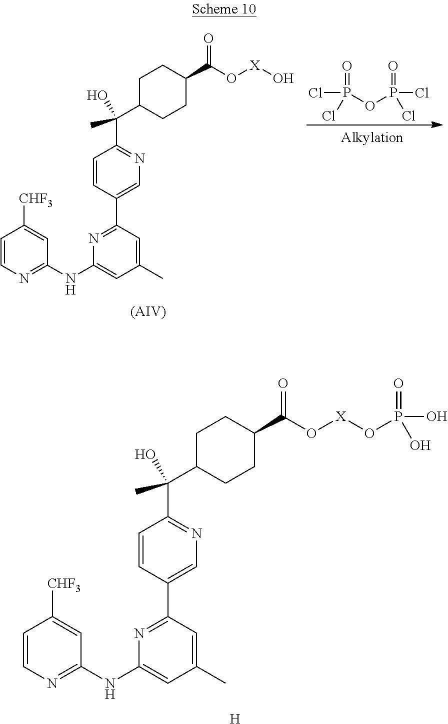

As shown in Scheme 10, phosphate containing compounds of structural subtype H are prepared from the trans-cyclohexane carboxylic acid derivatives (AIV) that contain an alcohol (wherein X is a linking group) by reaction with phosphorylating reagents and subsequent hydrolysis or deprotection reactions to afford free phosphate containing compounds of structural subtype H.

The preparations of compounds of Formula (I) are further exemplified by the following specific examples. The compounds exemplified are illustrative of the invention and are not, however, to be construed as limiting the scope of the invention in any manner. The examples further illustrate details for the preparation of the compounds of the present invention. Those skilled in the art will readily understand that known variations of protecting groups, of reagents, as well as of the conditions and processes of the following preparative procedures, can be used to prepare these compounds. It is also understood that whenever a chemical reagent is not commercially available, such a chemical reagent can be readily prepared by those skilled in the art by either following or adapting known methods described in the literature. All temperatures are degrees Celsius unless otherwise noted.

Nuclear magnetic resonance (NMR) spectra of routine samples were acquired on a Varian VNMRS 500 MHz spectrometer, using VNMRJ 2.1B software. The spectral window used was from 14 to −2 ppm, digitized to give a resolution of 0.3 Hz. The signal-to-noise ratio of spectra was typically 50:1 or better. Spectra of flow samples were acquired on a Varian Inova 600 MHz spectrometer using VNMRJ 2.1B software, and equipped with a Protasis microflow probe (cell volume 10 μL). Presaturation of residual solvent and water resonances was used. The spectral window was 14 to −2 ppm, digitized to produce 0.5 Hz resolution. Signal-to-noise ratio was 25:1 or better. All spectra were processed with 0.2 Hz line broadening. Liquid chromatography-mass spectrometry (LC-MS) spectra were recorded using either an Agilent Technologies 1200 series liquid chromatography system coupled to a 6120 single quadrupole mass spectrometer or an Agilent Technologies 1290 infinity series liquid chromatography system coupled to a 6150 single quadrupole mass spectrometer.

### EXAMPLES

### Preparative Example 1

### Preparation of Trans-4-[(1R)-(6-{[4-(Difluoromethyl)pyridin-2-yl]amino}-4-methyl-2,3′-bipyridin-6′-yl)-1-hydroxyethyl]cyclohexanecarboxylic acid

### Preparative Example 1-1

### 6-Bromo-N-[4-(difluoromethyl)pyridin-2-yl]-4-methylpyridin-2-amine

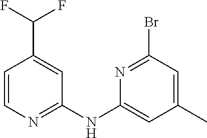 [SMILES: Cc1cc(Br)nc(Nc2cc(C(F)F)ccn2)c1]

Potassium t-butoxide (1.0 M in THF, 198 mL, 198 mmol) was added to a solution of 6-bromo-4-methyl pyridine-2-amine (37 g, 198 mmol) and 2-chloro-4-(difluoromethyl)pyridine (42.1 g, 257 mmol) in THF (60 mL) at 0° C. The resulting mixture was heated to reflux for 30 minutes then cooled to 0° C., and a second portion of potassium t-butoxide (1.0 M in THF, 80 mL, 80 mmol) was added. The mixture was again heated to reflux for 30 minutes, cooled to 0° C., and a third portion of potassium t-butoxide (1.0 M in THF, 80 mL, 80 mmol) was added. The mixture was again heated to reflux for 30 minutes. After cooling to 0° C., a fourth portion of potassium t-butoxide (1.0 M in THF, 20 mL, 20 mmol) was added. Upon refluxing for 30 minutes, the reaction was allowed to cool to room temperature, then was diluted with saturated aqueous NH₄Cl solution (500 mL) and DCM (500 mL). The layers were separated, and the aqueous layer was extracted a second time with DCM (500 mL). The combined organic layers were dried with sodium sulfate, filtered through a pad of CELITE (150 g), and concentrated under reduced pressure. The residue was triturated with DCM (100 mL), filtered, and washed with hexanes (2×50 mL) to afford one portion of 6-bromo-N-[4-(difluoromethyl)pyridin-2-yl]-4-methylpyridin-2-amine. The filtrate was concentrated, absorbed on silica gel and purified via silica gel column chromatography (ethyl acetate/Hex) to afford a second portion of 6-bromo-N-[4-(difluoromethyl)pyridin-2-yl]-4-methylpyridin-2-amine.

MS ESI calc'd. for C₁₂H₁₁BrF₂N₃ [M+H]⁺ 314 and 316. found 314 and 316. ¹H NMR (600 MHz, DMSO-d₆) δ 10.20 (s, 1H), 8.35 (d, J=5.1 Hz, 1H), 7.69 (s, 1H), 7.64 (s, 1H), 7.01 (d, J=5.1 Hz, 1H), 6.96 (t, J=22.3 Hz, 1H), 6.95 (s, 1H), 2.24 (s, 3H).

### Preparative Example 1-2

### Methyl trans-4-acetylcyclohexanecarboxylate

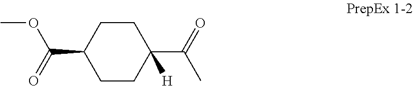

To a cooled solution (0° C.) under nitrogen of trans-4-(methoxycarbonyl)cyclohexanecarboxylic acid (18.9 g, 83 mmol) in DCM (150 mL) was added a catalytic amount of DMF (0.25 mL) followed by oxalyl chloride (7.97 mL, 91 mmol). The reaction mixture was then allowed to slowly warm to room temperature where it was stirred for 18 hours at which point it was concentrated to a yellow oil and dried under vacuum for 3 hours. To this residue (consisting primarily of methyl trans-4-(chlorocarbonyl)cyclohexanecarboxylate) was added [1,1′-Bis(diphenylphosphino)ferrocene]dichloropalladium(II)-DCM adduct (3.38 g, 4.14 mmol) and toluene (200 mL) and this reaction mixture was cooled in an ice bath. To this mixture was added dimethyl zinc (1.2 M in toluene, 81 mL, 97 mmol) at such a rate that the internal temperature did not exceed 15° C. The cooling bath was then removed and after 3 hours of stirring at room temperature the reaction mixture was re-cooled to 0° C. where it was diluted carefully with water. After the initial exotherm had subsided, sufficient 1M HCl and ethyl acetate were introduced such that a biphasic mixture formed. The layers were separated, the aqueous extracted a second time with ethyl acetate, and then the combined organics were dried with magnesium sulfate, filtered and concentrated under reduced pressure. The residue was purified by flash chromatography on silica to afford methyl trans-4-acetylcyclohexanecarboxylate. ¹H NMR (600 MHz, CDCl₃) δ 3.64 (s, 3H), 2.30 (tt, J=3.5, 12.1 Hz, 1H), 2.23 (tt, J=3.6, 12.1 Hz, 1H), 2.12 (s, 3H), 2.08-2.02 (m, 2H), 1.98-1.92 (m, 2H), 1.47-1.38 (m, 2H), 1.36-1.28 (m, 2H).

### Preparative Example 1-3

### Methyl trans-4-[1-(5-bromopyridin-2-yl)-1-hydroxyethyl]cyclohexanecarboxylate

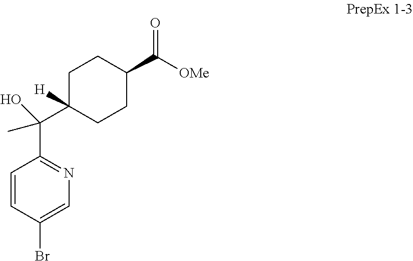 [SMILES: COC(=O)[C@H]1CC[C@H](C(C)(O)c2ccc(Br)cn2)CC1]

To a solution of 5-bromo-2-iodopyridine (26.3 g, 85 mmol) in DCM (180 mL) at 0° C. was slowly added a solution of isopropylmagnesium chloride in THF (44 g, 90 mmol) at such a rate that the internal temperature did not exceed 5° C. The reaction mixture was allowed to stir for 40 minutes at 0° C. To a solution of methyl trans-4-acetylcyclohexanecarboxylate (21.7 g, 81 mmol, PrepEx 1-2) in THF (93 mL) was added lanthanum(III) chloride bis(lithium chloride) complex solution (0.6 M in THF, 27.0 mL, 16.2 mmol). This mixture was allowed to stir for 30 minutes before cooling to 0° C. The aryl Grignard reagent solution was then added dropwise to the ketone solution at 0° C. over 45 minutes. After 90 minutes at 0° C., methanol (5 mL) was added and the reaction mixture was allowed to warm to room temperature overnight. The reaction mixture was diluted with water (100 mL), aqueous hydrochloric acid (2 M, 45 mL), and methanol (50 mL), the layers were separated, and the aqueous layer was extracted with ethyl acetate (50 mL). The combined organic layers were washed with half-saturated aqueous sodium bicaronate solution (150 mL) and water (100 mL) and then concentrated under reduced pressure. The residue was crystallized using ethyl acetate/heptane to afford racemic methyl trans-4-[1-(5-bromopyridin-2-yl)-1-hydroxyethyl]cyclohexane carboxylate as a yellow oil. MS ESI calc'd. for C₁₅H₂₁BrNO₃ [M+H]⁺ 342 and 344. found 342 and 344. ¹H NMR (600 MHz, CDCl₃) δ 8.54 (d, J=1.8 Hz, 1H), 7.79 (dd, J=2.3, 8.4 Hz, 1H), 7.19 (d, J=8.4 Hz, 1H), 4.65 (s, 1H), 3.61 (s, 3H), 2.16 (m, 1H), 2.05-1.97 (m, 1H), 1.97-1.90 (m, 1H), 1.90-1.83 (m, 1H), 1.56 (m, 1H), 1.45 (s, 3H), 1.37 (m, 1H), 1.31-1.15 (m, 3H), 1.09 (m, 1H).

### Preparative Example 1-3a and 1-3b

### Separation of methyl trans-4-[(1R)-1-(5-bromopyridin-2-yl)-1-hydroxyethyl]cyclohexane carboxylate and methyl trans-4-[(1S)-1-(5-bromopyridin-2-yl)-1-hydroxyethyl]cyclohexanecarboxylate

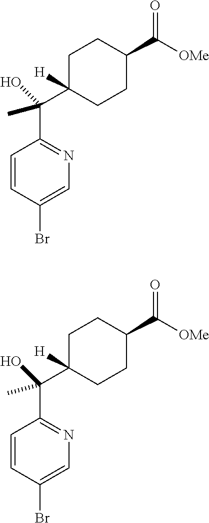 [SMILES: COC(=O)[C@H]1CC[C@H]([C@@](C)(O)c2ccc(Br)cn2)CC1 ; COC(=O)[C@H]1CC[C@H]([C@](C)(O)c2ccc(Br)cn2)CC1]

A racemic mixture of methyl trans-4-[1-(5-bromopyridin-2-yl)-1-hydroxyethyl]cyclohexane carboxylate was separated by chiral SFC purification [Thar 350 preparative SFC, ChiralPak AD-10 um, 300×50 mm I.D., 40% EtOH/CO₂ mobile phase, sample dissolved in MeOH ˜300 mg/mL, 4.5 mL per injection] to afford methyl trans-4-[(1R)-1-(5-bromopyridin-2-yl)-1-hydroxyethyl]cyclohexane carboxylate and methyl trans-4-[(1S)-1-(5-bromopyridin-2-yl)-1-hydroxyethyl]cyclohexanecarboxylate as single enantiomers.

### PrepEx 1-3a

Faster eluting enantiomer (R): MS ESI calc'd for C₁₅H₂₁BrNO₃ [M+H⁺ 342 and 344. found 342 and 344.

### PrepEx 1-3b

Slower eluting enantiomer (S): MS ESI calc'd for C₁₅H₂₁BrNO₃ [M+H⁺ 342 and 344. found 342 and 344.

### Preparative Example 1-4

### Methyl trans-4-[(1R)-(6-{[4-(difluoromethyl)pyridin-2-yl]amino}-4-methyl-2,3′-bipyridin-6′-yl)-1-hydroxyethyl]cyclohexanecarboxylate

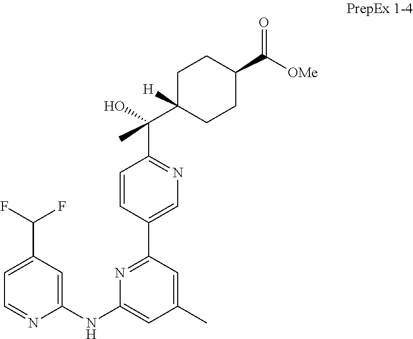 [SMILES: COC(=O)[C@H]1CC[C@H]([C@@](C)(O)c2ccc(-c3cc(C)cc(Nc4cc(C(F)F)ccn4)n3)cn2)CC1]

To a nitrogen degassed solution of methyl trans-4-[(1R)-(5-bromopyridin-2-yl)-1-hydroxyethyl]cyclohexane carboxylate (35 g, 102 mmol, PrepEx 1-3a, faster eluting isomer from chiral SFC separation of the racemate) in dioxane (350 mL) was added potassium acetate (20.1 g, 205 mmol), bis(pinacolate)diborane (28.6 g, 112 mmol), and [1,1-bis(diphenylphosphino)ferrocene]dichloropalladium(II)-dichloromethane complex (4.2 g, 5.11 mmol). The mixture was degassed by sparging with nitrogen for 30 minutes and the reaction was heated to 90° C. for 2 hours. The reaction mixture was allowed to cool to room temperature. 6-Bromo-N-[4-(difluoromethyl)pyridin-2-yl]-4-methylpyridin-2-amine (32.8 g, 104 mmol) was added followed by aqueous potassium carbonate (2 M, 128 mL, 256 mmol). The resulting mixture was degassed by sparging for 5 minutes with nitrogen and heated at 90° C. for 2.5 hours. The reaction was allowed to cool to room temperature, then diluted with water (50 mL) and ethyl acetate (50 mL). The reaction was filtered through a pad of CELITE. The residue was washed with water (200 mL) and ethyl acetate (200 mL). The filtered suspension was transferred into a separatory funnel. The layers were separated, and the aqueous layer was washed with Ethyl acetate (2×150 mL). The organic layers were combined, dried with MgSO₄, filtered, and concentrated in vacuo to afford a light red oil. The oil was absorbed on silica gel and purified via silica gel column chromatography (ethyl acetate/hexanes). Fractions containing the desired product were concentrated, reabsorbed on silica gel and purified again via silica gel column chromatography (ethyl acetate/hexanes). The desired product fractions were concentrated under reduced pressure. The residue was dissolved in ethyl acetate (1.5 L) and stirred for 1 hour with DARCO 174 KB-G (50 g). The mixture was filtered through a pad of CELITE (200 g), and the residue was washed with ethyl acetate (1500 mL). The filtrate was concentrated under reduced pressure to afford methyl trans-4-[(1R)-(6-{[4-(difluoromethyl)pyridin-2-yl]amino}-4-methyl-2,3′-bipyridin-6′-yl)-1-hydroxyethyl]cyclohexanecarboxylate.

MS ESI calc'd. for C₂₇H₃₁F₂N₄O₃ [M+H]⁺ 497. found 497.

¹H NMR (600 MHz, CDCl₃) δ 9.05 (d, J=1.5 Hz, 1H), 8.35 (d, J=5.1 Hz, 1H), 8.33 (dd, J=2.2, 8.3 Hz, 1H), 8.15 (s, 1H), 7.51 (s, 1H), 7.36 (d, J=8.3 Hz, 1H), 7.19 (s, 1H), 7.09 (s, 1H), 6.97 (d, J=5.1 Hz, 1H), 6.63 (t, J=56.0 Hz, 1H), 5.24 (s, 1H), 3.61 (s, 3H), 2.40 (s, 3H), 2.18 (tt, J=3.4, 12.3 Hz, 1H), 2.10-1.97 (m, 2H), 1.92-1.83 (m, 1H), 1.64 (tt, J=3.2, 11.8 Hz, 1H), 1.51 (s, 3H), 1.46-1.35 (m, 1H), 1.33-1.20 (m, 3H), 1.15 (m, 1H).

### Preparative Example 1-5

### trans-4-[(1R)-(6-{[4-(Difluoromethyl)pyridin-2-yl]amino}-4-methyl-2,3′-bipyridin-6′-yl)-1-hydroxyethyl]cyclohexanecarboxylic acid

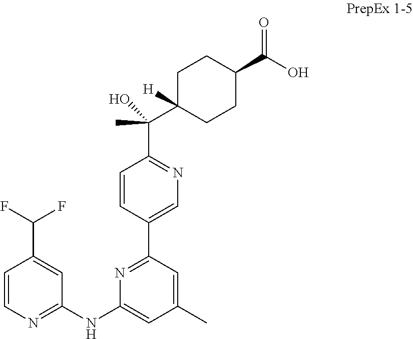 [SMILES: Cc1cc(Nc2cc(C(F)F)ccn2)nc(-c2ccc([C@](C)(O)[C@H]3CC[C@H](C(=O)O)CC3)nc2)c1]

To a solution of methyl trans-4-[(1R)-(6-{[4-(difluoromethyl)pyridin-2-yl]amino}-4-methyl-2,3′-bipyridin-6′-yl)-1-hydroxyethyl]cyclohexanecarboxylate (18.66 g, 37.6 mmol, PrepEx 1-4) from the previous step in methanol (186 mL) was slowly added aqueous sodium hydroxide (1 M, 132 mL, 132 mmol). An exotherm (˜15° C.) was observed and the reaction changed from a yellow solution to a milky white heterogenous mixture. The reaction was then heated to 65° C. for 1 hour. The reaction was allowed to cool to 50° C., and aqueous hydrochloric acid (1 M, 132 mL, 132 mmol) was added via addition funnel (˜30 min). Crystallization occurred upon neutralization and stirring was continued for 1 hour. The reaction was filtered at 30° C. The residue was washed with water (2×250 mL) and dried under nitrogen to give a solid. The solid was transferred to a flask, diluted with ethyl acetate (170 mL) and heated to 65° C. with stirring for 2 hours. Hexanes (170 mL) were added, and the mixture was allowed to cool to room temperature and aged for 1 hour. The mixture was filtered, the residue was washed with hexanes (170 mL) and dried under nitrogen to afford trans-4-[(1R)-(6-{[4-(difluoromethyl)pyridin-2-yl]amino}-4-methyl-2,3′-bipyridin-6′-yl)-1-hydroxyethyl]cyclohexanecarboxylic acid as a white solid. MS ESI calc'd. for C₂₆H₂₉F₂N₄O₃ [M+H]⁺ 483. found 483. ¹H NMR (500 MHz, CD₃OD) δ 9.16 (s, 1H), 8.44 (d, J=8.4 Hz, 1H), 8.34 (d, J=5.2 Hz, 1H), 8.28 (s, 1H), 7.71 (d, J=8.4 Hz, 1H), 7.36 (s, 1H), 7.30 (s, 1H), 7.02 (d, J=5.2 Hz, 1H), 6.82 (t, J=55.8 Hz, 1H), 2.42 (s, 3H), 2.20-1.79 (m, 5H), 1.56 (s, 3H), 1.47-1.14 (m, 5H).

### Preparative Example 2

### Preparation of Chloromethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

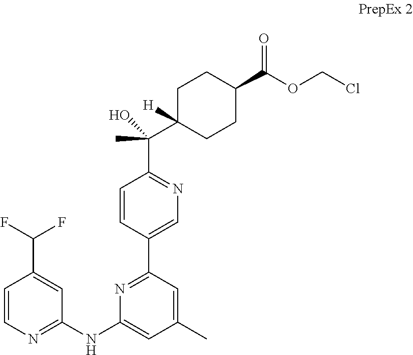 [SMILES: Cc1cc(Nc2cc(C(F)F)ccn2)nc(-c2ccc([C@](C)(O)[C@H]3CC[C@H](C(=O)OCCl)CC3)nc2)c1]

To a solution of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (5.00 g, 10.4 mmol, PrepEx 1-5) in DMF (100 mL) was added cesium carbonate (3.38 mg, 10.4 mmol). The reaction mixture was stirred for 30 minutes before bromochloromethane (100 mL, 1.49 mol) was added over 2 minutes. After 20 hours the reaction was diluted with diethyl ether (300 mL), ethyl acetate (200 mL), and water (200 mL). The layers were separated and the organic layer was washed with water (3×200 mL) and brine (50 mL), dried over sodium sulfate, filtered, and concentrated under reduced pressure. The residue was purified by chromatography on silica gel (30-100% ethyl acetate/hexanes) to provide chloromethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. ESI calc'd. for C₂₇H₃₀ClF₂N₄O₃ [M+H]⁺ 531. found 531. ¹H NMR (500 MHz, DMSO_{d-6}) δ 10.00 (s, 1H), 9.13 (d, J=2.2 Hz, 1H), 8.41-8.35 (m, 2H), 8.32 (s, 1H), 7.67 (d, J=8.3 Hz, 1H), 7.41 (d, J=5.5, 1H), 7.32 (s, 1H), 7.09 (d, J=55.6 Hz, 1H), 7.02 (d, J=5.1 Hz, 1H), 5.80 (s, 2H), 5.09 (s, 1H), 2.34 (s, 3H), 2.23-2.19 (m, 1H), 2.00-1.92 (m, 1H), 1.88-1.80 (m, 2H), 1.78-1.70 (m, 1H), 1.43 (s, 3H), 1.36-1.03 (m, 5H).

### Preparative Example 3

### Preparation of 2-Hydroxyethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

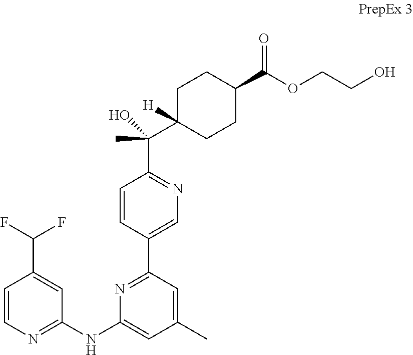 [SMILES: Cc1cc(Nc2cc(C(F)F)ccn2)nc(-c2ccc([C@](C)(O)[C@H]3CC[C@H](C(=O)OCCO)CC3)nc2)c1]

To a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (150 mg, 0.311 mmol, PrepEx 1-5), 2-hydroxyethyl acetate (162 mg, 1.55 mmol), and triphenylphosphine (resin-bound, 1.6 mmol/g loading, 389 mg, 0.622 mmol) in THF (3 mL) was added di-tert-butyl azodicarboxylate (143 mg, 0.622 mmol) at 20° C. The reaction mixture was stirred at 20° C. for 16 hours. The reaction mixture was diluted with TFA (1 mL) and water (1 drop). The mixture was stirred for 30 minutes. The mixture was then filtered through CELITE, washing with dichloromethane (3×10 mL). The filtrate was concentrated under reduced pressure to afford the crude residue TFA salt. The residue was diluted carefully with saturated aqueous sodium bicarbonate solution (25 mL) and ethyl acetate (100 mL). The organic layer was separated, washed with brine (25 mL), dried over magnesium sulfate, filtered, and concentrated under reduced pressure to afford the crude residue free base. The residue was purified by silica gel chromatography (0-100% ethyl acetate/hexanes, followed by 0-10% methanol/ethyl acetate). The purified material was dissolved in acetonitrile (2 mL) and diluted with water (6 mL). The resulting suspension was frozen and lyophilized to afford 2-hydroxyethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₂₈H₃₃F₂N₄O₄ [M+H]⁺ 527. found 527. ¹H NMR (500 MHz, DMSO-d₆) δ 10.00 (s, 1H), 9.13 (d, J=2.0 Hz, 1H), 8.39-8.35 (m, 2H), 8.32 (s, 1H), 7.68 (d, J=8.0 Hz, 1H), 7.42 (s, 1H), 7.33 (s, 1H), 7.09 (t, J=56.0 Hz, 1H), 7.02 (d, J=5.0 Hz, 1H), 5.06 (s, 1H), 4.74 (t, J=5.5 Hz, 1H), 3.96 (t, J=5.0 Hz, 2H), 3.53-3.48 (m, 2H), 2.34 (s, 3H), 2.14-2.06 (m, 1H), 1.96-1.90 (m, 1H), 1.86-1.70 (m, 3H), 1.43 (s, 3H), 1.32-1.09 (m, 5H).

### Example 1

### Preparation of Esters Using the General Methods Illustrated in Scheme 3

### Method A—Alkylation

### Example 1-1

### 2-Hydroxy-2-methylpropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

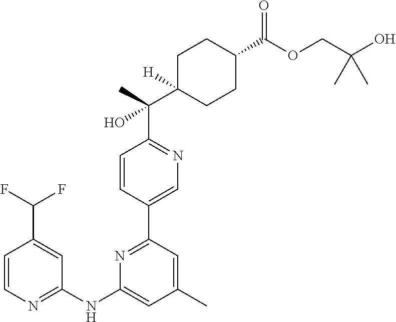 [SMILES: Cc1cc(Nc2cc(C(F)F)ccn2)nc(-c2ccc([C@](C)(O)[C@H]3CC[C@H](C(=O)OCC(C)(C)O)CC3)nc2)c1]

To a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (0.100 g, 0.21 mmol), potassium carbonate (0.057 g, 0.41 mmol), and sodium iodide (6 mg, 0.04 mmol) in DMF (1 mL) was added 1-chloro-2-methylpropan-2-ol (0.045 g, 0.41 mmol) at 20° C. The reaction mixture was heated at 70° C. for 2 hours, after which time analysis by LCMS indicated no conversion of starting material to desired product. The reaction mixture was heated at 100° C. for an additional 16 hours, after which time analysis by LCMS indicated partial conversion to desired product. The reaction mixture was heated at 120° C. for an additional 6 hours. LCMS indicated further conversion to desired product. Additional 1-chloro-2-methylpropan-2-ol (0.090 g, 0.83 mmol) and potassium carbonate (0.228 g, 1.66 mmol) were added and the reaction mixture was heated for an additional hour at 120° C. The reaction mixture was cooled to ambient temperature and diluted with ethyl acetate (100 mL). The mixture was washed with water (3×20 mL) and brine (1×15 mL). The organic layer was separated, dried over magnesium sulfate, filtered, and concentrated under reduced pressure. The residue was purified by silica gel chromatography (0-100% ethyl acetate/hexanes). The purified material was dissolved in acetonitrile (10 mL) and then diluted with water (20 mL). The resulting suspension was frozen and lyophilized to afford 2-hydroxy-2-methylpropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₃₀H₃₇F₂N₄O₄ [M+H]⁺ 555. found 555. ¹H NMR (500 MHz, DMSO-d₆) δ 10.00 (s, 1H), 9.13 (d, J=2.0 Hz, 1H), 8.39-8.35 (m, 2H), 8.32 (s, 1H), 7.68 (d, J=8.5 Hz, 1H), 7.42 (s, 1H), 7.32 (s, 1H), 7.09 (t, J=56 Hz, 1H), 7.02 (d, J=5.0 Hz, 1H), 5.06 (s, 1H), 4.54 (s, 1H), 3.73 (s, 2H), 2.34 (s, 3H), 2.16-2.10 (m, 1H), 1.97-1.92 (m, 1H), 1.86-1.79 (m, 2H), 1.78-1.70 (m, 1H), 1.44 (s, 3H), 1.34-1.08 (m, 5H), 1.04 (s, 6H).

### Method B—Coupling Using the Reagent EDC

### Example 1-2

### cis-4-Aminocyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

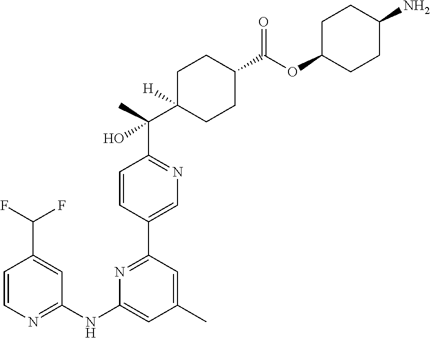 [SMILES: Cc1cc(Nc2cc(C(F)F)ccn2)nc(-c2ccc([C@](C)(O)[C@H]3CC[C@H](C(=O)O[C@H]4CC[C@@H](N)CC4)CC3)nc2)c1]

Step 1:

Di-tert-butyl azodicarboxylate (143 mg, 0.62 mmol) was added to a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (150 mg, 0.31 mmol), tert-butyl (trans-4-hydroxycyclohexyl)carbamate (335 mg, 1.55 mmol) and triphenylphosphine (Resin-bound, 1.6 mmol/g) (389 mg, 0.62 mmol) in THF (3.1 mL). The reaction mixture was stirred at room temperature for 48 hours. The reaction mixture was diluted with TFA (1 mL) and water (1 drop) and allowed to stir for 30 minutes. The mixture was then filtered through CELITE, washing with DCM (3×10 mL). The filtrate was concentrated under reduced pressure to afford the crude residue TFA salt. The residue was diluted with ethyl acetate and carefully quenched with aqueous sodium bicarbonate solution. The layers were separated and the aqueous layer was extracted with ethyl acetate. The combined organic fractions were washed with brine, dried over anhydrous sodium sulfate, filtered and concentrated under reduced pressure. The residue was purified by column chromatography on silica gel (0-100% ethyl acetate/hexanes) to afford cis-4-[(tert-butoxycarbonyl)amino]cyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₃₇H₄₈F₂N₅O₅ [M+H]⁺ 680. found 680.

Step 2:

TFA (1.0 mL, 13.0 mmol) was added to a mixture of cis-4-[(tert-butoxycarbonyl)amino]cyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (90 mg, 0.13 mmol) and dichloromethane (1.0 mL) and the resulting reaction mixture was stirred at room temperature for 30 minutes. The reaction mixture was concentrated under reduced pressure. The resulting residue was diluted with DMSO (1 mL) and purified by HPLC Reverse phase (acetonitrile/water+0.1% TFA). The fractions containing product were combined and diluted with ethyl acetate and aqueous sodium bicarbonate solution. The layers were separated and the organic layer was washed with brine, dried over anhydrous sodium sulfate, filtered and concentrated under reduced pressure to afford cis-4-aminocyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₃₂H₄₀F₂N₅O₃ [M+H]⁺ 580. found 580. ¹H NMR (500 MHz, DMSO-d₆) δ 10.0 (s, 1H), 9.13 (d, J=1.5 Hz, 1H), 8.39-8.32 (m, 3H), 7.68 (d, J=8.5 Hz, 1H), 7.42 (s, 1H), 7.32 (s, 1H), 7.10 (t, J=55.5 Hz, 1H), 7.03 (d, 4.5 Hz, 1H), 5.07 (s, 1H), 4.54 (s, 1H), 2.74-2.68 (m, 1H), 2.34 (s, 3H), 2.12-2.04 (m, 1H), 1.98-1.90 (m, 1H), 1.86-1.78 (m, 2H), 1.77-1.70 (m, 3H), 1.58-1.51 (m, 2H), 1.50-1.45 (m, 3H), 1.44 (s, 3H), 1.35-1.26 (m, 3H), 1.26-1.20 (m, 2H), 1.89-1.06 (m, 3H).

### EDC Coupling and Acetonide Removal

### Example 1-3

### (2S)-2,3-Dihydroxypropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

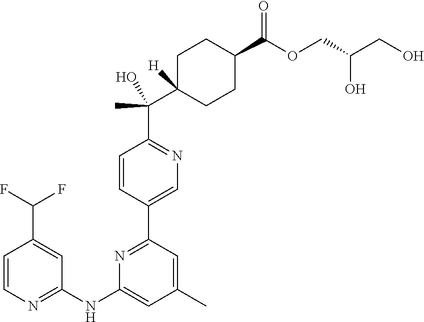 [SMILES: Cc1cc(Nc2cc(C(F)F)ccn2)nc(-c2ccc([C@](C)(O)[C@H]3CC[C@H](C(=O)OC[C@@H](O)CO)CC3)nc2)c1]

Step 1:

To a suspension of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (10.00 g, 20.72 mmol), [(4R)-2,2-dimethyl-1,3-dioxolan-4-yl]methanol (2.74 g, 20.72 mmol), and DMAP (0.633 g, 5.18 mmol) in THF (200 mL) was added EDC (4.77 g, 24.87 mmol). Triethylamine (3.47 mL, 24.9 mmol) and N,N-dimethylacetamide (10 mL) were added to the reaction mixture at 1 and 2 hours, respectively. After 18 hours, the reaction mixture was concentrated under reduced pressure to remove much of the THF and then it was diluted with diethyl ether (100 mL), ethyl acetate (200 mL), aqueous phosphate buffer (2 M, pH 7, 50 mL), and water (50 mL). The top organic layer was isolated and washed with aqueous phosphate buffer (2 M, pH 7, 75 mL), dilute aqueous sodium bicarbonate solution (4×(95 mL water+5 mL saturated aqueous sodium bicarbonate solution)), and brine (25 mL). The organic layer was dried over sodium sulfate, filtered, and concentrated under reduced pressure. The residue was purified by chromatography on silica gel (25-55% ethyl acetate/hexanes) to afford [(4S)-2,2-dimethyl-1,3-dioxolan-4-yl]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. ESI calc'd. for C₃₂H₃₉F₂N₄O₅ [M+H]⁺597. found 597. ¹H NMR (500 MHz, DMSO_{d-6}) δ 10.00 (s, 1H), 9.13 (d, J=1.7 Hz, 1H), 8.43-8.34 (m, 2H), 8.32 (s, 1H), 7.68 (d, J=8.3 Hz, 1H), 7.42 (s, 1H), 7.33 (s, 1H), 7.09 (d, J=55.5 Hz, 1H), 7.02 (d, J=4.8 Hz, 1H), 5.06 (s, 1H), 4.22-4.18 (m, 1H), 4.04 (dd, J=4.2, 11.6 Hz, 1H), 4.00-3.89 (m, 2H), 3.61 (dd, J=6.2, 8.4 Hz, 1H), 2.34 (s, 3H), 2.16-2.09 (m, 1H), 1.95-1.90 (m, 1H), 1.88-1.78 (m, 2H), 1.78-1.70 (m, 1H), 1.43 (s, 3H), 1.36-1.05 (m, 1H), 1.23-1.05 (m, 10H).

Step 2:

A solution of [(4S)-2,2-dimethyl-1,3-dioxolan-4-yl]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (8.25 g, 13.83 mmol) in TFA (50 mL) and water (5 mL) was stirred at 20° C. for 1 hour before toluene (4 mL) was added and the mixture was concentrated under reduced pressure to near dryness. More toluene (4 mL) was added and again the mixture was concentrated under reduced pressure to near dryness. The residue was diluted with 10% IPA/CHCl₃ (200 mL), water (30 mL), saturated aqueous sodium carbonate solution (30 mL), and saturated aqueous sodium bicarbonate solution (30 mL) (CAUTION: GAS EVOLUTION). The layers were separated, affording an aqueous layer with a pH of 9. The organic layer was washed with water (2×50 mL) and brine (50 mL), dried over sodium sulfate, filtered, and concentrated under reduced pressure. The residue was purified by chromatography on silica gel (1-10% methanol/dichloromethane) to afford a white solid, which was further purified through precipitation from dichlormethane/methanol/heptanes. The solid was filtered and dried under nitrogen to provide (2S)-2,3-Dihydroxypropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate.

ESI calc'd. for C₂₉H₃₅F₂N₄O₅ [M+H]⁺ 557. found 557.

### EDC Coupling and Benzyl Deprotection

### Example 1-4

### 6-O-({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)-D-glucopyranose

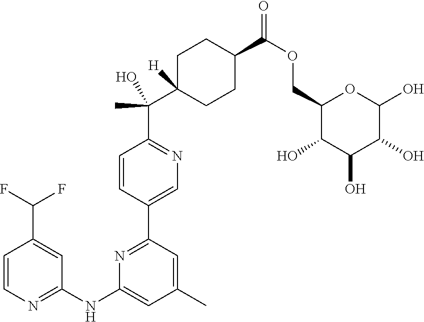 [SMILES: Cc1cc(Nc2cc(C(F)F)ccn2)nc(-c2ccc([C@](C)(O)[C@H]3CC[C@H](C(=O)OC[C@H]4OC(O)[C@H](O)[C@@H](O)[C@@H]4O)CC3)nc2)c1]

Step 1:

To a flask containing trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (900 mg, 1.87 mmol), benzyl 2,3,4-tri-O-benzyl-D-glucopyranoside (Tet. 2007, 63, 10042) (1.00 g, 1.87 mmol), EDC (715 mg, 3.73 mmol) and DMAP (11 mg, 0.093 mmol) was added DMF (11 mL) and diisopropylethylamine (0.98 mL, 5.60 mmol) and the mixture was stirred overnight. The reaction was diluted with ethyl acetate and water. The organic layer was separated, dried over magnesium sulfate, filtered and concentrated under reduced pressure. The crude product was purified by silica gel chromatography to give benzyl 2,3,4-tri-O-benzyl-6-O-({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)-D-glucopyranoside. MS ESI calc'd for C₆₀H₆₃F₂N₄O₈ [M+H]⁺ 1006. found 1006.

Step 2:

To a mixture of benzyl 2,3,4-tri-O-benzyl-6-O-({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)-D-glucopyranoside (280 mg, 0.28 mmol) in dichloromethane (9 mL) at −78° C. was added boron tribromide (1 M in DCM, 1.4 mL, 1.4 mmol). The mixture was allowed to warm to room temperature and then stirred for 30 minutes. The mixture was diluted with water and then with ethyl acetate. The organic layer was separated, dried over magnesium sulfate, filtered and concentrated under reduced pressure. The crude residue was purified by reverse phase HPLC (10-100% acetonitrile/water gradient with a 0.1% TFA modifier) to afford 6-O-({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)-D-glucopyranose. MS ESI calc'd for C₃₂H₃₉F₂N₄O₈ [M+H]⁺ 646. found 646.

### Method C—Mitsunobu-Type Coupling

### Example 1-5

### trans-3-Aminocyclobutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

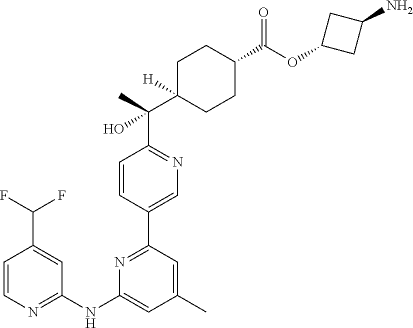 [SMILES: Cc1cc(Nc2cc(C(F)F)ccn2)nc(-c2ccc([C@](C)(O)[C@H]3CC[C@H](C(=O)O[C@H]4C[C@H](N)C4)CC3)nc2)c1]

Step 1:

A solution of (3-oxo-cyclobutyl)-carboxylic acid (20 g, 175 mmol) and triethylamine (30 mL, 215 mmol) in 1:1 THF-toluene (150 mL) was treated with diphenyl phosphoryl azide (38 mL, 175 mmol). The solution was warmed to 60° C. over 45 minutes, at which point nitrogen evolution was noted. After 3 hours, benzyl alcohol (18 mL, 175 mmol) was added and the solution was kept at 60° C. for 4 hours. After cooling to room temperature, the solution was diluted with ethyl acetate, washed with saturated aqueous sodium bicarbonate, 0.5 M HCl (2×), saturated aqueous sodium bicarbonate, dried over anhydrous magnesium sulfate, filtered, and concentrated under reduced pressure. The crude product was recrystallized from ethyl acetate and petroleum ether to afford benzyl (3-oxocyclobutyl)carbamate.

Step 2:

To a 0° C. solution of benzyl (3-oxocyclobutyl)carbamate (11.5 g, 52.5 mmol) in ethanol (50 mL) was added portionwise sodium borohydride (1.0 g, 26.4 mmol). The reaction mixture was stirred at room temperature until complete conversion was achieved. The mixture was concentrated under reduced pressure. The residue was taken up in dichloromethane and treated with saturated sodium bicarbonate solution. The phases were separated and the aqueous phase extracted with dichloromethane (2×). The organic phases were combined, dried over magnesium sulfate and concentrated under reduced pressure. The residue was purified by silica chromatography to afford a mixture of benzyl (cis-3-hydroxycyclobutyl)carbamate and benzyl (trans-3-hydroxycyclobutyl)carbamate. Benzyl (cis-3-hydroxycyclobutyl)carbamate was purified by recrystallization from ethanol. MS ESI calc'd. for C₁₂H₁₆NO₃ [M+H]⁺ 222. found 222. ¹H NMR (300 MHz, CD₃OD): δ 7.34-7.32 (m, 5H), 5.04 (s, 2H), 3.96-3.88 (m, 1H), 3.66-3.56 (m, 1H), 2.68-2.59 (m, 2H), 1.85-1.76 (m, 2H).

The mother liquid was concentrated and purified by prep-HPLC to afford benzyl (trans-3-hydroxycyclobutyl)carbamate. ¹H NMR (300 MHz, CD₃OD): δ 7.34-7.28 (m, 5H), 5.05 (s, 2H), 4.39-4.29 (m, 1H), 4.21-4.10 (m, 1H), 2.24-2.22 (m, 4H).

Step 3:

To a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (200 mg, 0.41 mmol), benzyl (cis-3-hydroxycyclobutyl)carbamate (388 mg, 2.07 mmol) and triphenylphosphine (Resin-bound, 1.6 mmol/g) (518 mg, 0.83 mmol) in THF (4.1 mL) was added di-tert-butyl azodicarboxylate (191 mg, 0.83 mmol). The reaction mixture was stirred at room temperature for 18 hours. The reaction mixture was diluted with TFA (1 mL) and water (1 drop) and allowed to stir for 30 minutes. The mixture was then filtered through CELITE, washing with DCM (3×10 mL). The filtrate was concentrated under reduced pressure to afford the crude residue TFA salt. The residue was diluted with ethyl acetate and carefully quenched with aqueous sodium bicarbonate solution. The aqueous layer was extracted with ethyl acetate. The combined organic fractions were washed with brine, dried over sodium sulfate, filtered and concentrated under reduced pressure. The residue was purified by column chromatography on silica gel, (0-100% ethyl acetate/hexanes) to afford trans-3-{[(benzyloxy)carbonyl]amino}cyclobutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate as a colorless oil. MS ESI calc'd. for C₃₈H₄₂F₂N₅O₅ [M+H]⁺ 686. found 686.

Step 4:

To a flask was added trans-3-{[(benzyloxy)carbonyl]amino}cyclobutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (196 mg, 0.29 mmol) and Pd/C (3 mg, 0.03 mmol). The flask was evacuated and backfilled with nitrogen 5 times. Ethanol (5.7 mL) was added and the flask was evacuated and backfilled with nitrogen 5 times. A balloon of hydrogen was attached and the mixture was stirred under a blanket of hydrogen at room temperature for 18 hours. The reaction was filtered through CELITE, washing with methanol, and the filtrate was concentrated under reduced pressure. The resulting oil was taken up in acetonitrile and water, frozen, and lyophilized to afford trans-3-aminocyclobutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₃₀H₃₆F₂N₅O₃ [M+H]⁺ 552. found 552. ¹H NMR (500 MHz, DMSO-d₆) δ 10.0 (s, 1H), 9.13 (d, J=1.5 Hz, 1H), 8.39-8.30 (m, 3H), 7.68 (d, J=1.5 Hz, 1H), 7.42 (s, 1H), 7.33 (s, 1H), 7.09 (t, J=55.5 Hz, 1H), 7.02 (d, J=5.0 Hz, 1H), 4.98-4.91 (m, 1H), 3.49-3.43 (m, 1H), 2.34 (s, 3H), 2.12-2.00 (m, 3H), 1.99-1.87 (m, 2H), 1.86-1.80 (m, 2H), 1.80-1.69 (m, 3H), 1.64-1.56 (m, 1H), 1.43 (s, 3H), 1.30-1.18 (m, 3H), 1.18-1.08 (m, 3H).

Method D—Coupling using the Reagent PyBrop

### Example 1-6

### Phenyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

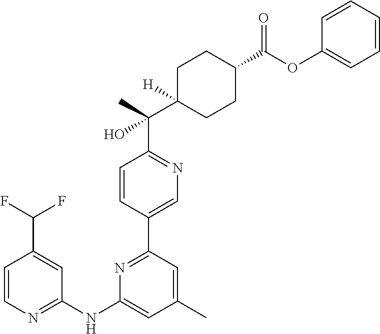 [SMILES: Cc1cc(Nc2cc(C(F)F)ccn2)nc(-c2ccc([C@](C)(O)[C@H]3CC[C@H](C(=O)Oc4ccccc4)CC3)nc2)c1]

To a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (500 mg, 1.04 mmol), phenol (195 mg, 2.07 mmol), and PyBrop (483 mg, 1.04 mmol) in THF (5 mL) was added Hunig's base (0.54 mL, 3.1 mmol) at 20° C. After 16 hours, the reaction mixture was diluted with water (25 mL) and ethyl acetate (100 mL). The organic layer was separated, washed with brine (2×25 mL), dried over magnesium sulfate, filtered, and concentrated under reduced pressure. The residue was purified by silica gel chromatography (0-100% ethyl acetate/hexanes). The resulting product was dissolved in acetonitrile (4 mL) and then diluted with water (8 mL). The resulting suspension was frozen and lyophilized to afford phenyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₃₂H₃₃F₂N₄O₃ [M+H]⁺ 559. found 559. ¹H NMR (500 MHz, DMSO-d₆) δ 10.00 (s, 1H), 9.15 (d, J=2.0 Hz, 1H), 8.39-8.35 (m, 2H), 8.33 (s, 1H), 7.70 (d, J=8.5 Hz, 1H), 7.43 (s, 1H), 7.37 (t, J=7.5 Hz, 2H), 7.33 (s, 1H), 7.24-7.19 (m, 1H), 7.10-6.98 (m, 4H), 5.11 (s, 1H), 2.42-2.35 (m, 1H), 2.35 (s, 3H), 2.14-2.08 (m, 1H), 2.00-1.94 (m, 1H), 1.93-1.87 (m, 1H), 1.83-1.76 (m, 1H), 1.46 (s, 3H), 1.45-1.36 (m, 1H), 1.32-1.26 (m, 4H).

The compounds in the following table were prepared by using methods similar to those described above in Methods A-D of Example 1.

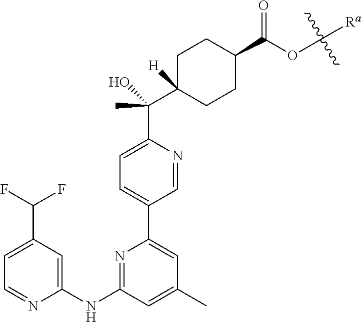

| Ex. | | | [M + H]⁺ | [M + H]⁺ | | |
|---|---|---|---|---|---|---|
| No. | R^{a} | Name | Calc'd | Obsv'd | Form(s) | Method¹ |
| 1-7 | 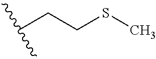 [SMILES: *CCSC] | 2- (methylsulfanyl)ethyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 557 | 557 | Free Base | B |
| 1-8 | 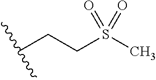 [SMILES: *CCS(C)(=O)=O] | 2- (methylsulfonyl)ethyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 589 | 589 | Free Base | B |
| 1-9 | 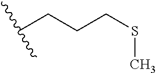 [SMILES: *CCCSC] | 3- (methylsulfanyl)propyl trans-4-[(1R)-1-(6- {[4- (difluoromethyl)pyridin- 2-yl]amino}-4- methyl-2,3′-bipyridin- 6′-yl)-1- hydroxyethyl] cyclohexanecarboxylate | 571 | 571 | Free Base | B |
| 1-10 | 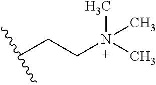 | 2-[({trans-4-[(1R)-1- (6-{[4- (difluoromethyl)pyridin- 2-yl]amino}-4- methyl-2,3′-bipyridin- 6′-yl)-1- hydroxyethyl]cyclohexyl} carbonyl)oxy]- N,N,N- trimethylethanaminium | 568* | 568* | TFA Salt | B |
| 1-11 | 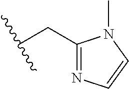 [SMILES: *Cc1nccn1C] | (1-methyl-1H- imidazol-2-yl)methyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 577 | 577 | TFA Salt | B |
| 1-12 | 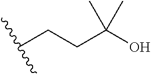 [SMILES: *CCC(C)(C)O] | 3-hydroxy-3- methylbutyl trans-4- [(1R)-1-(6-{[4- (difluoromethyl)pyridin- 2-yl]amino}-4- methyl-2,3′-bipyridin- 6′-yl)-1- hydroxyethyl] cyclohexanecarboxylate | 569 | 569 | Free Base | B |
| 1-13 | 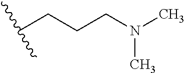 [SMILES: *CCCN(C)C] | 3- (dimethylamino)propyl trans-4-[(1R)-1-(6- {[4- (difluoromethyl)pyridin- 2-yl]amino}-4- methyl-2,3′-bipyridin- 6′-yl)-1- hydroxyethyl] cyclohexanecarboxylate | 568 | 568 | Free Base | C |
| 1-14 | 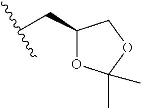 [SMILES: *C[C@H]1COC(C)(C)O1] | [(4R)-2,2-dimethyl- 1,3-dioxolan-4- yl]methyl trans-4- [(1R)-1-(6-{[4- (difluoromethyl)pyridin- 2-yl]amino}-4- methyl-2,3′-bipyridin- 6′-yl)-1- hydroxyethyl] cyclohexanecarboxylate | 597 | 597 | Free Base | C |
| 1-15 | 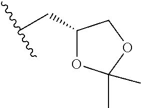 [SMILES: *C[C@@H]1COC(C)(C)O1] | [(4S)-2,2-dimethyl- 1,3-dioxolan-4- yl]methyl trans 4- [(1R)-1-(6-{[4- (difluoromethyl)pyridin- 2-yl]amino}-4- methyl-2,3′-bipyridin- 6′-yl)-1- hydroxyethyl] cyclohexanecarboxylate | 597 | 597 | Free Base | B |
| 1-16² | 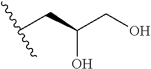 [SMILES: *C[C@H](O)CO] | (2R)-2,3- dihydroxypropyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 557 | 557 | Free Base | C |
| 1-17³ | 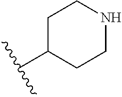 [SMILES: *C1CCNCC1] | piperidin-4-yl trans-4- [(1R)-1-(6-{[4- (difluoromethyl)pyridin- 2-yl]amino}-4- methyl-2,3′-bipyridin- 6′-yl)-1- hydroxyethyl] cyclohexanecarboxylate | 566 | 566 | Free Base | C |
| 1-18³ | 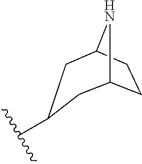 [SMILES: *C1CC2CCC(C1)N2] | 8- azabicyclo[3.2.1]oct- 3-yl trans-4-[(1R)-1- (6-{[4- (difluoromethyl)pyridin- 2-yl]amino}-4- methyl-2,3′-bipyridin- 6′-yl)-1- hydroxyethyl] cyclohexanecarboxylate | 592 | 592 | Free Base | C |
| 1-19⁴ | 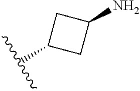 [SMILES: *[C@H]1C[C@H](N)C1] | trans-3- aminocyclobutyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 552 | 552 | Free Base | C |
| 1-20 | 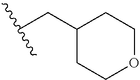 [SMILES: *CC1CCOCC1] | tetrahydro-2H-pyran- 4-ylmethyl trans-4- [(1R)-1-(6-{[4- (difluoromethyl)pyridin- 2-yl]amino}-4- methyl-2,3′-bipyridin- 6′-yl)-1- hydroxyethyl] cyclohexanecarboxylate | 581 | 581 | Free Base | C |
| 1-21³ | 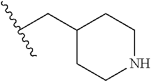 [SMILES: *CC1CCNCC1] | piperidin-4-ylmethyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 580 | 580 | Free Base | C |
| 1-22³ | 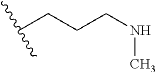 [SMILES: *CCCNC] | 3- (methylamino)propyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 554 | 554 | Free Base | C |
| 1-23³ | 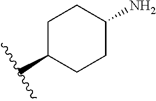 [SMILES: *[C@H]1CC[C@H](N)CC1] | trans-4- aminocyclohexyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 580 | 580 | Free Base | C |
| 1-24³ | 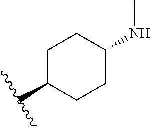 [SMILES: *[C@H]1CC[C@H](NC)CC1] | trans-4- (methylamino)cyclohexyl trans-4-[(1R)-1-(6- {[4- (difluoromethyl)pyridin- 2-yl]amino}-4- methyl-2,3′-bipyridin- 6′-yl)-1- hydroxyethyl] cyclohexanecarboxylate | 594 | 594 | Free Base | C |
| 1-25 | 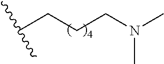 | 6- (dimethylamino)hexyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 610 | 610 | TFA Salt | B |
| 1-26 | 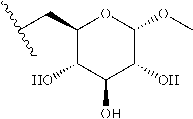 [SMILES: *C[C@H]1O[C@H](OC)[C@H](O)[C@@H](O)[C@@H]1O] | methyl 6-O-({trans-4- [(1R)-1-(6-{[4- (difluoromethyl)pyridin- 2-yl]amino}-4- methyl-2,3′-bipyridin- 6′-yl)-1- hydroxyethyl]cyclohexyl} carbonyl)-alpha- D-glucopyranoside | 659 | 659 | Free Base | C |
| 1-27 | 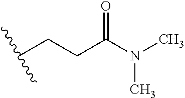 [SMILES: *CCC(=O)N(C)C] | 3-(dimethylamino)-3- oxopropyl trans-4- [(1R)-1-(6-{[4- (difluoromethyl)pyridin- 2-yl]amino}-4- methyl-2,3′-bipyridin- 6′-yl)-1- hydroxyethyl] cyclohexanecarboxylate | 582 | 582 | Free Base | C |
| 1-28 | 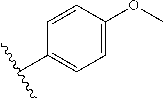 [SMILES: *c1ccc(OC)cc1] | 4-methoxyphenyl trans-4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 589 | 589 | Free Base | D |
| 1-29 | 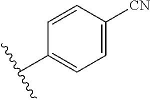 [SMILES: *c1ccc(C#N)cc1] | 4-cyanophenyl trans- 4-[(1R)-1-(6-{[4- (difluoromethyl)pyridin- 2-yl]amino}-4- methyl-2,3′-bipyridin- 6′-yl)-1- hydroxyethyl] cyclohexanecarboxylate | 584 | 584 | Free Base | D |

¹Method B refers to the experimental conditions described in Example 1-2; Method C refers to the experimental conditions described in Example 1-5; Method C refers to the experimental conditions described in Example 1-6
²Synthesized through a combination of EDC coupling and acetonide deprotection as in Example 1-3.
³Final step involved a Boc deprotection as in Example 1-2, Step 2.
⁴Final step involved a Cbz deprotection as in Example 1-5, Step 4.
*Parent ion calculated and determined as [M⁺].

### Example 2

### Preparation of Ester Acetals Using the General Method Illustrated in Scheme 4

### Example 2-1

### ({[(6-Aminohexyl)oxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

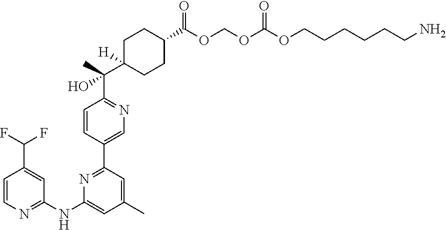 [SMILES: Cc1cc(Nc2cc(C(F)F)ccn2)nc(-c2ccc([C@](C)(O)[C@H]3CC[C@H](C(=O)OCOC(=O)OCCCCCCN)CC3)nc2)c1]

Step 1:

To a solution of tert-butyl(6-hydroxyhexyl)carbamate (420 mg, 1.93 mmol) in DCM (20 mL) at −10° C. was added chloromethyl carbonochloridate (0.22 mL, 2.5 mmol). After stirring for 10 minutes at −10° C., a solution of pyridine (0.47 mL, 5.8 mmol) in DCM (5 mL) was added dropwise over 10 minutes. After the addition was complete, the reaction mixture was allowed to warm to ambient temperature and was stirred for an additional 16 hours. The reaction mixture was then concentrated under reduced pressure. The residue was purified by silica gel chromatography (0-100% ethyl acetate in hexanes, linear gradient) to afford tert-butyl (6-(((chloromethoxy)carbonyl)oxy)hexyl)carbamate. MS ESI calc'd. for C₁₃H₂₄ClNO₅Na [M+Na]⁺ 332. found 332. ¹H NMR (500 MHz, DMSO-d₆) δ 6.76 (t, J=5.0 Hz, 1H), 5.87 (s, 2H), 4.15 (t, J=6.5 Hz, 2H), 2.90-2.83 (m, 2H), 1.62-1.56 (m, 2H), 1.38-1.21 (m, 6H), 1.35 (s, 9H).

Step 2:

To a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (470 mg, 0.97 mmol), potassium carbonate (404 mg, 2.92 mmol), and sodium iodide (29 mg, 0.20 mmol) was added a solution of tert-butyl (6-(((chloromethoxy)carbonyl)oxy)hexyl)carbamate (453 mg, 1.46 mmol) in DMF (5 mL) at 20° C. The resulting suspension was stirred for 24 hours at 20° C. The reaction mixture was then diluted with ethyl acetate (200 mL) and washed with water (2×50 mL) and brine (1×25 mL). The organic layer was separated, dried over magnesium sulfate, filtered, and concentrated under reduced pressure. The residue was purified by silica gel chromatography (0-100% ethyl acetate/hexanes) to afford 14,14-dimethyl-3,12-dioxo-2,4,13-trioxa-11-azapentadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₃₉H₅₂F₂N₅O₈ [M+H]⁺ 756. found 756.

Step 3:

To a solution of 14,14-dimethyl-3,12-dioxo-2,4,13-trioxa-11-azapentadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (660 mg, 0.87 mmol) in dichloromethane (10 mL) was added TFA (2.0 mL, 26 mmol). The reaction mixture was stirred for 3 hours at 20° C. The reaction mixture was concentrated under reduced pressure. The residue was azeotroped with acetonitrile (2×15 mL) and then diluted with acetonitrile (10 mL) and water (20 mL), frozen, and lyophilized to afford the desired product. The isolated product residue was dissolved again in acetonitrile (15 mL), diluted with water (40 mL), frozen, and lyophilized to afford ({[(6-aminohexyl)oxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. Product was isolated as a TFA salt.

MS ESI calc'd. for C₃₄H₄₄F₂N₅O₆ [M+H]⁺ 656. found 656. ¹H NMR (500 MHz, DMSO-d₆) δ 10.13 (s, 1H), 9.14 (d, J=2.0 Hz, 1H), 8.55 (d, J=8.0 Hz, 1H), 8.40 (d, J=5.0 Hz, 1H), 8.25 (s, 1H), 7.81 (d, J=8.5 Hz, 1H), 7.64 (s, 3H), 7.49 (s, 1H), 7.38 (s, 1H), 7.10 (t, J=55.5 Hz, 1H), 7.05 (d, J=5.0 Hz, 1H), 5.64 (s, 2H), 4.10 (t, J=6.5 Hz, 2H), 2.78-2.70 (m, 2H), 2.36 (s, 3H), 2.24-2.16 (m, 1H), 1.97-1.90 (m, 1H), 1.90-1.72 (m, 3H), 1.60-1.54 (m, 2H), 1.52-1.45 (m, 5H), 1.34-1.10 (m, 9H).

The compounds in the following table were prepared by using methods similar to those described above in Example 2.

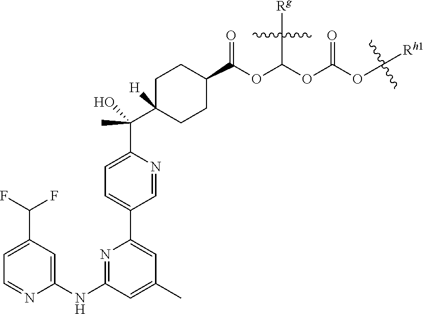

| Ex. | | | | [M + H]⁺ | [M + H]⁺ | |
|---|---|---|---|---|---|---|
| No. | R^{g} | R^{h1} | Name | Calc'd | Obsv'd | Form(s) |
| 2-2 | —H | 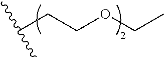 | 3-oxo-2,4,7,10- tetraoxadodec-1-yl trans-4-[(1R)-1-(6- {[4-(difluoromethyl) pyridin-2-yl]amino}-4- methyl-2,3′- bipyridin-6′-yl)-1- hydroxyethyl]cyclo hexanecarboxylate | 673 | 673 | Free Base, Chloride Salt |
| 2-3 | —H | 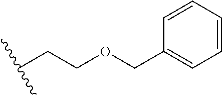 [SMILES: *CCOCc1ccccc1] | ({[2-(benzyloxy)ethoxy] carbonyl}oxy) methyl trans-4-[(1R)-1- (6-{[4-(difluoromethyl) pyridin-2-yl]amino}-4- methyl-2,3′- bipyridin-6′-yl)-1- hydroxyethyl]cyclo hexanecarboxylate | 691 | 691 | Free Base |
| 2-4 | —H | 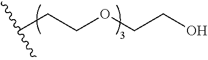 | 15-hydroxy-3-oxo- 2,4,7,10,13- pentaoxapentadec- 1-yl trans-4-[(1R)- 1-(6-{[4- (difluoromethyl) pyridin-2-yl]amino}-4- methyl-2,3′- bipyridin-6′-yl)-1- hydroxyethyl]cyclo hexanecarboxylate | 733 | 733 | Free Base |
| 2-5 | —H | 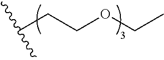 | 3-oxo-2,4,7,10,13- pentaoxapentadec- 1-yl trans-4-[(1R)- 1-(6-{[4- (difluoromethyl) pyridin-2-yl]amino}-4- methyl-2,3′- bipyridin-6′-yl)-1- hydroxyethyl]cyclo hexanecarboxylate | 717 | 717 | TFA Salt |
| 2-6 | —H | 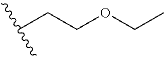 [SMILES: *CCOCC] | {[(2-ethoxyethoxy) carbonyl]oxy}methyl trans-4-[(1R)-1-(6- {[4-(difluoromethyl) pyridin-2-yl]amino}-4- methyl-2,3′- bipyridin-6′-yl)-1- hydroxyethyl]cyclo hexanecarboxylate | 629 | 629 | TFA Salt |
| 2-7 | —H | 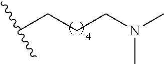 [SMILES: *CCCN(C)C] | [({[6-(dimethylamino) hexyl]oxy}carbonyl) oxy]methyl trans-4- [(1R)-1-(6-{[4- (difluoromethyl) pyridin-2-yl]amino}-4- methyl-2,3′- bipyridin-6′-yl)-1- hydroxyethyl]cyclo hexanecarboxylate | 684 | 684 | TFA Salt |
| 2-8 | —H | 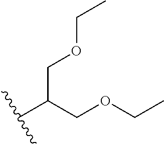 [SMILES: *C(COCC)COCC] | ({[2-ethoxy-1- (ethoxymethyl) ethoxy]carbonyl}oxy) methyl trans-4-[(1R)- 1-(6-{[4- (difluoromethyl) pyridin-2-yl]amino}-4- methyl-2,3′- bipyridin-6′-yl)-1- hydroxyethyl]cyclo hexanecarboxylate | 687 | 687 | Free Base |
| 2-9 | —H | 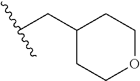 [SMILES: *CC1CCOCC1] | {[(tetrahydro-2H- pyran-4- ylmethoxy)carbonyl] oxy}methyl trans- 4-[(1R)-1-(6-{[4- (difluoromethyl) pyridin-2-yl]amino}-4- methyl-2,3′- bipyridin-6′-yl)-1- hydroxyethyl]cyclo hexanecarboxylate | 655 | 655 | Free Base |
| 2-10 | —H | 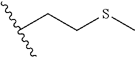 [SMILES: *CCSC] | ({[2-(methylsulfanyl) ethoxy]carbonyl}oxy) methyl trans-4- [(1R)-1-(6-{[4- (difluoromethyl) pyridin-2-yl]amino}-4- methyl-2,3′- bipyridin-6′-yl)-1- hydroxyethyl]cyclo hexanecarboxylate | 631 | 631 | Free Base |
| 2-11¹ | —H | 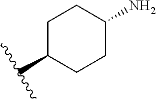 [SMILES: *[C@H]1CC[C@H](N)CC1] | ({[(trans-4- aminocyclohexyl) oxy]carbonyl}oxy) methyl trans-4-[(1R)- 1-(6-{[4- (difluoromethyl) pyridin-2-yl]amino}-4- methyl-2,3′- bipyridin-6′-yl)-1- hydroxyethyl]cyclo hexanecarboxylate | 654 | 654 | TFA Salt |
| 2-12 | —H | 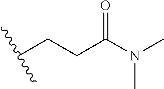 [SMILES: *CCC(=O)N(C)C] | ({[3-(dimethylamino)- 3-oxopropoxy] carbonyl}oxy)methyl trans-4-[(1R)-1-(6- {[4-(difluoromethyl) pyridin-2-yl]amino}-4- methyl-2,3′- bipyridin-6′-yl)-1- hydroxyethyl]cyclo hexanecarboxylate | 656 | 656 | Free Base |
| 2-13 | —H | 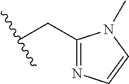 [SMILES: *Cc1nccn1C] | ({[(1-methyl-1H- imidazol-2- yl)methoxy]carbonyl} oxy)methyl trans-4-[(1R)-1-(6- {[4-(difluoromethyl) pyridin-2-yl]amino}-4- methyl-2,3′- bipyridin-6′-yl)-1- hydroxyethyl]cyclo hexanecarboxylate | 651 | 651 | Free Base |
| 2-14 | —CH₃ | 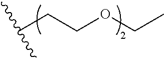 | 1-methyl-3-oxo- 2,4,7,10- tetraoxadodec-1-yl trans-4-[(1R)-1-(6- {[4-(difluoromethyl) pyridin-2-yl]amino}-4- methyl-2,3′- bipyridin-6′-yl)-1- hydroxyethyl]cyclo hexanecarboxylate | 687 | 687 | Free Base |
| 2-15 | —CH₃ | 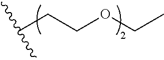 | 1-methyl-3-oxo- 2,4,7,10- tetraoxadodec-1-yl trans-4-[(1R)-1-(6- {[4-(difluoromethyl) pyridin-2-yl]amino}-4- methyl-2,3′- bipyridin-6′-yl)-1- hydroxyethyl]cyclo hexanecarboxylate | 687 | 687 | Free Base |
| 2-16 | —C(H)(CH₃)₂ | 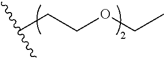 | 1-(1-methylethyl)- 3-oxo-2,4,7,10- tetraoxadodec-1-yl trans-4-[(1R)-1-(6- {[4-(difluoromethyl) pyridin-2-yl]amino}-4- methyl-2,3′- bipyridin-6′-yl)-1- hydroxyethyl]cyclo hexanecarboxylate | 715 | 715 | Free Base |
| 2-17 | —C(H)(CH₃)₂ | 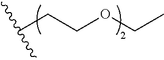 | 1-(1-methylethyl)- 3-oxo-2,4,7,10- tetraoxadodec-1-yl trans-4-[(1R)-1-(6- {[4-(difluoromethyl) pyridin-2-yl]amino}-4- methyl-2,3′- bipyridin-6′-yl)-1- hydroxyethyl]cyclo hexanecarboxylate | 715 | 715 | Free Base |

¹Final step included removal of the Boc protecting group as in Example 2-1, Step 3.

### Example 3

### Preparation of Compounds Using the General Method Illustrated in Scheme 5

### Example 3-1

### [({Trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl oxy]methyl piperidine-4-carboxylate

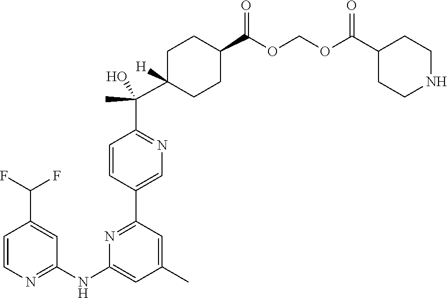 [SMILES: Cc1cc(Nc2cc(C(F)F)ccn2)nc(-c2ccc([C@](C)(O)[C@H]3CC[C@H](C(=O)OCOC(=O)C4CCNCC4)CC3)nc2)c1]

Step 1:

To a solution of 1-(tert-butoxycarbonyl)piperidine-4-carboxylic acid (1.38 g, 4.97 mmol) in DMF (15 mL) was added cesium carbonate (1.499 g, 4.60 mmol). After 30 minutes, bromochloromethane (15 mL, 224 mmol) was added over 3 minutes. The reaction mixture was diluted with diethyl ether (100 mL), ethyl acetate (50 mL) and water (25 mL) after stirring for 20 hours and then the layers were separated. The organic layer was washed with water (4×50 mL) and brine, dried over sodium sulfate, filtered, and concentrated under reduced pressure to provide 1-tert-butyl 4-(chloromethyl) piperidine-1,4-dicarboxylate, which was used without further purification.

¹H NMR (500 MHz, CDCl₃) δ 5.72 (s, 2H), 4.06 (br s, 2H), 2.91-2.84 (m, 2H), 2.59-2.44 (m, 1H), 1.93-1.86 (m, 1H), 1.69-1.61 (m, 2H), 1.45 (s, 9H).

Step 2:

To a solution of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (1.00 g, 2.07 mmol) in DMF (8 mL) was added potassium carbonate (573 mg, 4.14 mmol). After 10 minutes, sodium iodide (311 mg, 2.07 mmol) was added followed by 1-tert-butyl 4-(chloromethyl) piperidine-1,4-dicarboxylate (677 mg, 2.072 mmol) as a solution in DMF (4 mL) after another 20 minutes. The reaction mixture was diluted with diethyl ether (100 mL), ethyl acetate (50 mL), saturated aqueous sodium bicarbonate solution (25 mL), and water (20 mL) after 20 hours and then the layers were separated. The organic layer was washed with dilute aqueous sodium bicarbonate solution (4×50 mL (water (47 mL) plus saturated aqueous sodium bicarbonate solution (3 mL)) and brine (25 mL), dried over sodium sulfate, filtered, and concentrated under reduced pressure. The residue was purified by chromatography on silica gel (40-70% ethyl acetate/hexanes) to give 1-tert-butyl 4-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}piperidine-1,4-dicarboxylate. ESI calc'd. for C₃₈H₄₈F₂N₅O₇ [M+H]⁺ 724. found 724. ¹H NMR (500 MHz, CDCl₃) δ 9.07 (d, J=1.9 Hz, 1H), 8.41-8.31 (m, 2H), 8.17 (s, 1H), 7.46 (s, 1H), 7.38 (d, J=8.3 Hz, 1H), 7.21 (s, 1H), 7.12 (s, 1H), 6.99 (d, J=5.1 Hz, 1H), 6.66 (t, J=56.0 Hz, 1H), 5.73 (s, 2H), 5.26 (s, 1H), 4.00 (br s, 2H), 2.84-2.78 (br m, 2H), 2.53-2.45 (m, 1H), 2.45 (s, 3H), 2.31-2.18 (m, 1H), 2.11-1.98 (m, 1H), 1.95-1.78 (m, 3H), 1.71-1.55 (m, 3H), 1.54 (s, 3H), 1.46-1.38 (m, 1H), 1.44 (s, 9H), 1.37-1.24 (m, 4H), 1.22-1.13 (m, 1H).

Step 3:

To a solution of 1-tert-butyl 4-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}piperidine-1,4-dicarboxylate (150 mg, 0.207 mmol) in DCM (4 mL) was added TFA (1 mL). After 30 minutes, toluene (1 mL) was added and then the reaction mixture was concentrated under reduced pressure. The residue was dissolved in a mixture of 10% IPA/CHCl₃ (25 mL), saturated aqueous sodium carbonate solution (5 mL), and 5 mL saturated aqueous sodium bicarbonate solution (5 mL). The layers were separated and the aqueous layer was extracted with 10% IPA/CHCl₃ (2×12 mL). The combined organic layers were dried over sodium sulfate, filtered, and concentrated under reduced pressure. The residue was lyophilized from acetonitrile and water to provide [({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl piperidine-4-carboxylate. ESI calc'd. for C₃₃H₄₀F₂N₅O₅ [M+H]⁺ 624. found 624. ¹H NMR (500 MHz, CDCl₃) δ 9.07 (d, J=2.0 Hz, 1H), 8.43-8.31 (m, 2H), 8.17 (s, 1H), 7.44 (s, 1H), 7.37 (d, J=8.3 Hz, 1H), 7.21 (s, 1H), 7.11 (s, 1H), 6.99 (d, J=5.2 Hz, 1H), 6.66 (t, J=56.0 Hz, 1H), 5.73 (s, 2H), 5.26 (s, 1H), 3.08 (dt, J=3.6, 12.4 Hz, 2H), 2.62 (td, J=2.7, 12.3 Hz, 2H), 2.48-2.43 (m, 1H), 2.43 (s, 3H), 2.24 (t, J=12.1 Hz, 1H), 2.05 (t, J=14.0 Hz, 2H), 1.94-1.80 (m, 3H), 1.70-1.55 (m, 3H), 1.53 (s, 3H), 1.46-1.40 (m, 1H), 1.37-1.23 (m, 4H), 1.21-1.15 (m, 1H).

### Example 3-2

### 1-[({Trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]ethyl 1-methyl-1H-pyrazole-4-carboxylate

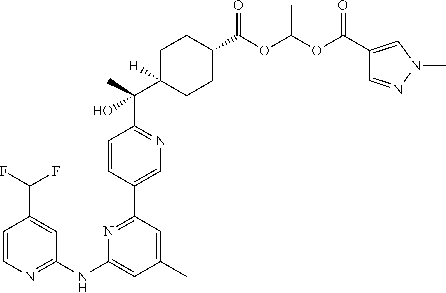 [SMILES: Cc1cc(Nc2cc(C(F)F)ccn2)nc(-c2ccc([C@](C)(O)[C@H]3CC[C@H](C(=O)OC(C)OC(=O)c4cnn(C)c4)CC3)nc2)c1]

Step 1:

To a suspension of 1-methyl-1H-pyrazole-4-carboxylic acid (300 mg, 2.38 mmol) in DCM (5 mL) at 20° C. was added oxalyl chloride (0.416 mL, 4.76 mmol) followed by DMF (0.018 mL, 0.238 mmol). The reaction mixture was stirred for 4 hours at 20° C. The reaction mixture was then concentrated under reduced pressure to afford 1-methyl-1H-pyrazole-4-carbonyl chloride. The crude product residue was used in the subsequent step without workup or purification.

Step 2:

A flask was charged with zinc chloride (32 mg, 0.23 mmol) and 1-methyl-1H-pyrazole-4-carbonyl chloride (344 mg, 2.38 mmol), and the flask was then degassed with vacuum and backfilled with argon. To this sealed flask was added DCM (25 mL) and the resulting suspension was cooled to −5° C. (ice/acetone bath). To this suspension was added acetaldehyde (0.148 mL, 2.62 mmol). The resulting suspension was stirred at −5° C. for 15 minutes, and then warmed to ambient temperature. The suspension was stirred at ambient temperature for an additional 18 hours. The reaction mixture was then diluted with water (25 mL) and stirred vigorously for 10 minutes. The reaction mixture was diluted with diethyl ether (100 mL). The organic layer was separated, dried over magnesium sulfate, filtered, and concentrated under reduced pressure to afford 1-chloroethyl 1-methyl-1H-pyrazole-4-carboxylate. The material was used in the subsequent step without purification. MS ESI calc'd. for C₇H₁₀ClN₂O₂ [M+H]⁺ 189. found 189. ¹H NMR (500 MHz, DMSO-d₆) δ 8.42 (s, 1H), 7.92 (s, 1H), 6.73 (q, J=5.5 Hz, 1H), 3.87 (s, 3H), 1.81 (d, J=6.0 Hz, 3H).

Step 3:

(Example 3-2): To a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (400 mg, 0.83 mmol), sodium iodide (25 mg, 0.17 mmol), and potassium carbonate (573 mg, 4.14 mmol) was added a solution of 1-chloroethyl 1-methyl-1H-pyrazole-4-carboxylate (235 mg, 1.24 mmol) in DMF (4 mL) at ambient temperature. The resulting suspension was heated to 75° C. and stirred for 5 hours. The reaction mixture was cooled to ambient temperature, and then diluted with ethyl acetate (150 mL) and water (50 mL). The organic layer was separated and then washed with saturated sodium carbonate solution (3×25 mL), water (2×25 mL), and brine (25 mL). The organic layer was separated, dried over magnesium sulfate, filtered, and concentrated under reduced pressure. The residue was purified by silica gel chromatography (0-100% (5% methanol in ethyl acetate)/hexanes). The product was dissolved in acetonitrile (10 mL) and diluted with water (30 mL). The resulting suspension was frozen and lyophilized to afford 1-[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]ethyl 1-methyl-1H-pyrazole-4-carboxylate (Example 3-2). MS ESI calc'd. for C₃₃H₃₇F₂N₆O₅ [M+H]⁺ 635. found 635. ¹H NMR (500 MHz, DMSO-d₆) δ 10.00 (s, 1H), 9.13 (s, 1H), 8.39-8.30 (m, 4H), 7.83 (d, J=6.5 Hz, 1H), 7.67 (d, J=8.0 Hz, 1H), 7.42 (s, 1H), 7.33 (s, 1H), 7.09 (t, J=55.5 Hz, 1H), 7.02 (d, J=4.5 Hz, 1H), 6.84 (q, J=5.0 Hz, 1H), 5.07 (s, 1H), 3.84 (app d, J=5.5 Hz, 3H), 2.34 (s, 3H), 2.16-2.10 (m, 1H), 1.96-1.87 (m, 1H), 1.86-1.70 (m, 3H), 1.47-1.40 (m, 6H), 1.30-1.08 (m, 5H).

Step 4:

A racemic mixture of 1-[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]ethyl 1-methyl-1H-pyrazole-4-carboxylate was separated by chiral SFC purification [Thar 350 preparative SFC, Chiralcel OJ-H, 21×250 mm, 25% (MeOH+0.25% dimethylethylamine)/CO₂ mobile phase, sample dissolved in MeOH ˜38 mg/mL, 0.25 mL per injection] to afford (1R)-1-[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]ethyl 1-methyl-1H-pyrazole-4-carboxylate and (1S)-1-[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]ethyl 1-methyl-1H-pyrazole-4-carboxylate as the single enantiomers.

### Example 3-2a

Faster eluting diastereomer: MS ESI calc'd. for C₃₃H₃₇F₂N₆O₅ [M+H]⁺ 635. found 635.

### Example 3-2b

Slower eluting diastereomer: MS ESI calc'd. for C₃₃H₃₇F₂N₆O₅ [M+H]⁺ 635. found 635.

### Example 3-3

### [(2-Hydroxy-2-methylpropanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

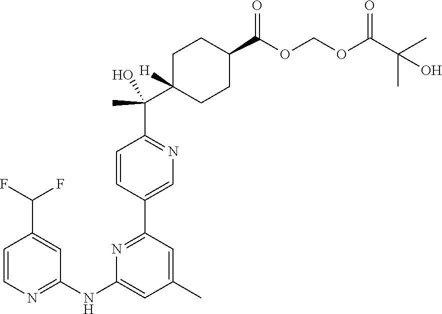 [SMILES: Cc1cc(Nc2cc(C(F)F)ccn2)nc(-c2ccc([C@](C)(O)[C@H]3CC[C@H](C(=O)OCOC(=O)C(C)(C)O)CC3)nc2)c1]

To a suspension of 2-hydroxy-2-methylpropanoic acid (4.9 mg, 0.041 mmol), sodium iodide (6.2 mg, 0.041 mmol), chloromethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (20 mg, 0.038 mmol) in acetonitrile (0.5 mL) was added triethylamine (6 al, 0.04 mmol). The reaction mixture was heated at 80° C. for 90 minutes and then allowed to cool to room temperature. Water (0.1 mL) and DMSO (0.5 mL) were added, the mixture was filtered with a syringe filter, and the filtrate was purified by reverse phase HPLC (acetonitrile/water with a 0.1% TFA modifier) to give [(2-hydroxy-2-methylpropanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. ESI calc'd. for C₃₁H₃₇F₂N₄O₆ [M+H]⁺ 599. found 599. ¹H NMR (500 MHz, DMSO_{d-6}) δ 10.18 (s, 1H), 9.14 (d, J=2.0 Hz, 1H), 8.66 (br s, 1H), 8.41 (d, J=5.2 Hz, 1H), 8.19 (s, 1H), 7.90 (br s, 1H), 7.54 (s, 1H), 7.41 (s, 1H), 7.10 (t, J=56 Hz, 1H), 7.06 (d, J=4.7 Hz, 1H), 5.68 (s, 2H), 2.40-2.35 (m, 4H), 2.23-2.17 (m, 1H), 1.93 (d, J=11.5 Hz, 1H), 1.87 (d, J=11.5 Hz, 1H), 1.80-1.71 (m, 2H), 1.50 (s, 6H), 1.34-1.05 (m, 7H).

The compounds in the following table were prepared by using methods similar to those described above in Example 3.

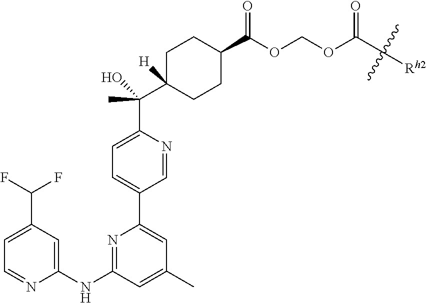

| Ex. | | | [M + H]⁺ | [M + H]⁺ | |
|---|---|---|---|---|---|
| No. | R^{h2} | Name | Calc′d | Obsv′d | Form(s) |
| 3-4 | 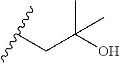 [SMILES: *CC(C)(C)O] | [(3-hydroxy-3- methylbutanoyl)oxy] methyl trans-4-[(1R)-1-(6-{[4- (difluoromethyl)pyridin-2- yl]amino}-4-methyl-2,3′- bipyridin-6′-yl)-1- hydroxyethyl]cyclohexane carboxylate | 613 | 613 | Free Base |
| 3-5 | 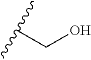 [SMILES: *CO] | [(hydroxyacetyl)oxy] methyl trans-4-[(1R)-1-(6-{[4- (difluoromethyl)pyridin-2- yl]amino}-4-methyl-2,3′- bipyridin-6′-yl)-1- hydroxyethyl]cyclohexane carboxylate | 571 | 571 | Free Base |
| 3-6 | 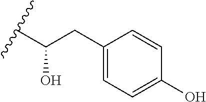 [SMILES: *[C@@H](O)Cc1ccc(O)cc1] | {[(2S)-2-hydroxy-3-(4- hydroxyphenyl)propanoyl] oxy}methyl trans-4-[(1R)- 1-(6-{[4- (difluoromethyl)pyridin-2- yl]amino}-4-methyl-2,3′- bipyridin-6′-yl)-1- hydroxyethyl]cyclohexane carboxylate | 677 | 677 | Free Base |
| 3-7 | 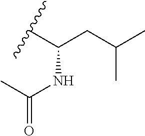 [SMILES: *[C@H](CC(C)C)NC(C)=O] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl N- acetyl-L-leucinate | 668 | 668 | Free Base |
| 3-8 | 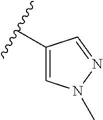 [SMILES: *c1cnn(C)c1] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl 1- methyl-1H-pyrazole-4- carboxylate | 621 | 621 | Free Base |
| 3-9 | 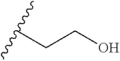 [SMILES: *CCO] | [(3-hydroxypropanoyl)oxy] methyl trans-4-[(1R)-1-(6- {[4-(difluoromethyl)pyridin-2- yl]amino}-4-methyl-2,3′- bipyridin-6′-yl)-1- hydroxyethyl]cyclohexane carboxylate | 585 | 585 | Free Base |
| 3-10 | 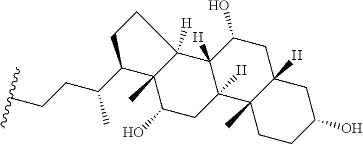 | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl (3alpha,7alpha,12alpha)- 3,7,12-trihydroxycholan- 24-oate | 903 | 903 | Free Base |
| 3-11 | 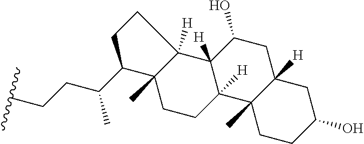 | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl (3alpha,7alpha)-3,7- dihydroxycholan-24-oate | 887 | 887 | Free Base |
| 3-12 | 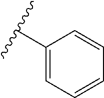 [SMILES: *c1ccccc1] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl benzoate | 617 | 617 | Free Base |
| 3-13 | 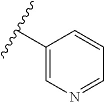 [SMILES: *c1cccnc1] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl pyridine-3-carboxylate | 618 | 618 | Free Base |
| 3-14 | 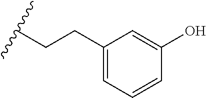 [SMILES: *CCc1cccc(O)c1] | {[3-(3- hydroxyphenyl)propanoyl] oxy}methyl trans-4-[(1R)- 1-(6-{[4- (difluoromethyl)pyridin-2- yl]amino}-4-methyl-2,3′- bipyridin-6′-yl)-1- hydroxyethyl]cyclohexane carboxylate | 661 | 661 | Free Base |
| 3-15 | 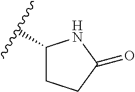 [SMILES: *[C@H]1CCC(=O)N1] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl 5- oxo-D-prolinate | 624 | 624 | Free Base |
| 3-16 | 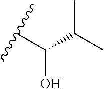 [SMILES: *[C@@H](O)C(C)C] | {[(2S)-2-hydroxy-3- methylbutanoyl]oxy} methyl trans-4-[(1R)-1-(6-{[4- (difluoromethyl)pyridin-2- yl]amino}-4-methyl-2,3′- bipyridin-6′-yl)-1- hydroxyethyl]cyclohexane carboxylate | 613 | 613 | Free Base |
| 3-17 | 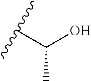 [SMILES: *[C@@H](C)O] | {[(2R)-2- hydroxypropanoyl]oxy} methyl trans-4-[(1R)-1-(6- {[4-(difluoromethyl)pyridin-2- yl]amino}-4-methyl-2,3′- bipyridin-6′-yl)-1- hydroxyethyl]cyclohexane carboxylate | 585 | 585 | Free Base |
| 3-18 | 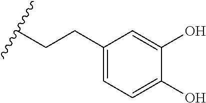 [SMILES: *CCc1ccc(O)c(O)c1] | {[3-(3,4- dihydroxyphenyl)propanoyl] oxy}methyl trans-4- [(1R)-1-(6-{[4- (difluoromethyl)pyridin-2- yl]amino}-4-methyl-2,3′- bipyridin-6′-yl)-1- hydroxyethyl]cyclohexane carboxylate | 677 | 677 | Free Base |
| 3-19 | 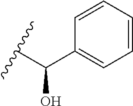 [SMILES: *[C@H](O)c1ccccc1] | {[(2R)-2-hydroxy-2- phenylacetyl]oxy}methyl trans-4-[(1R)-1-(6-{[4- (difluoromethyl)pyridin-2- yl]amino}-4-methyl-2,3′- bipyridin-6′-yl)-1- hydroxyethyl]cyclohexane carboxylate | 647 | 647 | Free Base |
| 3-20 | 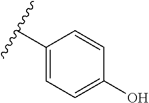 [SMILES: *c1ccc(O)cc1] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl 4- hydroxybenzoate | 633 | 633 | Free Base |
| 3-21 | 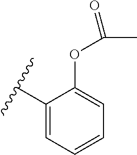 [SMILES: *c1ccccc1OC(C)=O] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl 2- (acetyloxy)benzoate | 675 | 675 | Free Base |
| 3-22 | 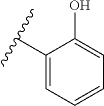 [SMILES: *c1ccccc1O] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl 2- hydroxybenzoate | 633 | 633 | Free Base |
| 3-23 | 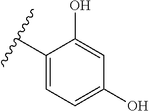 [SMILES: *c1ccc(O)cc1O] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl 2,5- dihydroxybenzoate | 649 | 649 | Free Base |
| 3-24 | 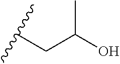 [SMILES: *CC(C)O] | [(3-hydroxybutanoyl)oxy] methyl trans-4-[(1R)-1-(6-{[4- (difluoromethyl)pyridin-2- yl]amino}-4-methyl-2,3′- bipyridin-6′-yl)-1- hydroxyethyl]cyclohexane carboxylate | 599 | 599 | Free Base |
| 3-25 | 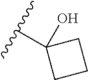 [SMILES: *C1(O)CCC1] | {[(1-hydroxycyclobutyl) carbonyl]oxy}methyl trans-4- [(1R)-1-(6-{[4- (difluoromethyl)pyridin-2- yl]amino}-4-methyl-2,3′- bipyridin-6′-yl)-1- hydroxyethyl]cyclohexane carboxylate | 611 | 611 | Free Base |
| 3-26 | 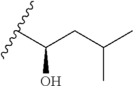 [SMILES: *[C@H](O)CC(C)C] | {[(2S)-2-hydroxy-4- methylpentanoyl]oxy}methyl trans-4-[(1R)-1-(6-{[4- (difluoromethyl)pyridin-2- yl]amino}-4-methyl-2,3′- bipyridin-6′-yl)-1- hydroxyethyl]cyclohexane carboxylate | 627 | 627 | Free Base |
| 3-27 | 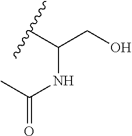 [SMILES: *C(CO)NC(C)=O] | [(N-acetylseryl)oxy]methyl trans-4-[(1R)-1-(6-{[4- (difluoromethyl)pyridin-2- yl]amino}-4-methyl-2,3′- bipyridin-6′-yl)-1- hydroxyethyl]cyclohexane carboxylate | 642 | 642 | Free Base |
| 3-28 | 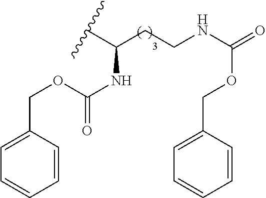 [SMILES: *[C@@H](CCNC(=O)OCc1ccccc1)NC(=O)OCc1ccccc1] | [({trans-4-[(1R)-1-(6-{[4- (difluoromethyl)pyridin-2- yl]amino}-4-methyl-2,3′- bipyridin-6′-yl)-1- hydroxyethyl]cyclohexyl} carbonyl)oxy]methyl N~2~,N~6~- bis[(benzyloxy)carbonyl]- L-lysinate | 909 | 909 | Free Base |
| 3-29 | 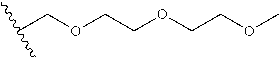 [SMILES: *COCCOCCOC] | 3-oxo-2,5,8,11- tetraoxadodec-1-yl trans- 4-[(1R)-1-(6-{[4- (difluoromethyl)pyridin-2- yl]amino}-4-methyl-2,3′- bipyridin-6′-yl)-1- hydroxyethyl]cyclohexane carboxylate | 673 | 673 | Free Base |

### Example 4

### Preparation of Ester Carbamates Using the General Method Illustrated in Scheme 6

### Example 4-1

### 1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (mixture of diastereomers), 1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (Diastereomer 1) and 1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (Diastereomer 2)

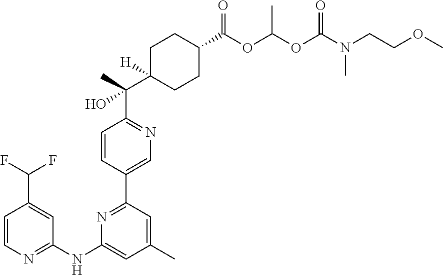 [SMILES: COCCN(C)C(=O)OC(C)OC(=O)[C@H]1CC[C@H]([C@@](C)(O)c2ccc(-c3cc(C)cc(Nc4cc(C(F)F)ccn4)n3)cn2)CC1]

Step 1:

To a solution of 2-methoxy-N-methylethanamine (330 mg, 3.7 mmol) in DCM (10 mL) at −10° C. was added 1-chloroethyl carbonochloridate (0.40 mL, 3.7 mmol). After stirring for 10 minutes at −10° C., a solution of pyridine (0.60 mL, 7.4 mmol) in dichloromethane (5 mL) was added dropwise over 10 minutes. After the addition was complete, the reaction mixture was allowed to warm to ambient temperature and stirred for an additional 16 hours. The reaction mixture was concentrated under reduced pressure. The residue was purified by silica gel chromatography (0-100% ethyl acetate/hexanes) to afford 1-chloroethyl(2-methoxyethyl)(methyl)carbamate. MS ESI calc'd. for C₇H₁₅ClNO₃ [M+H]⁺ 196. found 196. ¹H NMR (500 MHz, DMSO-d₆) δ 6.56-6.51 (m, 1H), 3.44-3.30 (m, 4H), 3.28 (app d, J=2.0 Hz, 3H), 2.87 (app d, J=5.5 Hz, 3H), 1.74 (app t, J=6.0 Hz, 3H).

Step 2:

(Example 4-1): To a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (600 mg, 1.24 mmol), potassium carbonate (516 mg, 3.73 mmol), and sodium iodide (37 mg, 0.25 mmol) was added a solution of 1-chloroethyl(2-methoxyethyl)(methyl)carbamate (414 mg, 2.11 mmol) in DMF (3 mL) at 20° C. The resulting suspension was stirred for 24 hours at 20° C. The reaction mixture was then diluted with ethyl acetate (200 mL) and washed with water (2×50 mL) and brine (1×50 mL). The organic layer was separated, dried over magnesium sulfate, filtered, and concentrated under reduced pressure. The residue was purified by silica gel chromatography (0-100% ethyl acetate/hexanes) to afford the product residue. The product residue was diluted with acetonitrile (15 mL) and water (30 mL). The resulting suspension was frozen and lyophilized to afford 1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate as a mixture of diastereomers (Example 4-1). MS ESI calc'd. for C₃₃H₄₂F₂N₅O₆ [M+H]⁺ 642. found 642. ¹H NMR (500 MHz, DMSO-d₆) δ 10.00 (s, 1H), 9.13 (d, J=2.5 Hz, 1H), 8.39-8.35 (m, 2H), 8.33 (s, 1H), 7.67 (d, J=8.5 Hz, 1H), 7.41 (s, 1H), 7.32 (s, 1H), 7.09 (t, J=55.5 Hz, 1H), 7.02 (d, J=5.5 Hz, 1H), 6.61-6.54 (m, 1H), 5.07 (s, 1H), 3.40-3.25 (m, 4H), 3.22-3.16 (m, 3H), 2.83-2.78 (m, 3H), 2.34 (s, 3H), 2.14-2.06 (m, 1H), 1.93-1.86 (m, 1H), 1.86-1.80 (m, 1H), 1.79-1.70 (m, 2H), 1.43 (s, 3H), 1.39-1.34 (m, 3H), 1.31-1.08 (m, 5H).

Step 3:

A solution of 1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate in methanol (5 mL) was separated into pure diastereomers by SFC chromatography [30% methanol in CO₂ with 0.25% dimethylethylamine on a Phenomenex Lux Cellulose-4 column (5 uM, 21×250 mm)].

### Example 4-1a

The faster eluting peak was concentrated under reduced pressure to afford the product residue. The product residue was diluted with acetonitrile (10 mL) and water (20 mL) and the resulting suspension was frozen and lyophilized to afford 1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (faster eluting diastereomer). MS ESI calc'd. for C₃₃H₄₂F₂N₅O₆ [M+H]⁺ 642. found 642. ¹H NMR (500 MHz, DMSO-d₆) δ 10.00 (s, 1H), 9.13 (d, J=2.0 Hz, 1H), 8.39-8.35 (m, 2H), 8.33 (s, 1H), 7.67 (d, J=8.5 Hz, 1H), 7.42 (s, 1H), 7.33 (s, 1H), 7.09 (t, J=55.5 Hz, 1H), 7.02 (d, J=5.0 Hz, 1H), 6.61-6.55 (m, 1H), 5.06 (s, 1H), 3.42-3.35 (m, 2H), 3.35-3.27 (m, 2H), 3.20 (app d, J=4.0 Hz, 3H), 2.81 (app d, J=7.5 Hz, 3H), 2.34 (s, 3H), 2.13-2.08 (m, 1H), 1.92-1.86 (m, 1H), 1.85-1.80 (m, 1H), 1.80-1.70 (m, 2H), 1.43 (s, 3H), 1.36 (app t, J=5.0 Hz, 3H), 1.30-1.08 (m, 5H).

### Example 4-1b

The slower eluting peak was concentrated under reduced pressure to afford the product residue. The product residue was diluted with acetonitrile (10 mL) and water (20 mL) and the resulting suspension was frozen and lyophilized to afford 1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (slower eluting diastereomer). MS ESI calc'd. for C₃₃H₄₂F₂N₅O₆ [M+H]⁺ 642. found 642. ¹H NMR (500 MHz, DMSO-d₆) δ 10.00 (s, 1H), 9.13 (d, J=2.0 Hz, 1H), 8.39-8.35 (m, 2H), 8.33 (s, 1H), 7.67 (d, J=8.5 Hz, 1H), 7.42 (s, 1H), 7.33 (s, 1H), 7.09 (t, J=55.5 Hz, 1H), 7.02 (d, J=5.0 Hz, 1H), 6.61-6.55 (m, 1H), 5.06 (s, 1H), 3.39-3.34 (m, 2H), 3.34-3.25 (m, 2H), 3.18 (app d, J=6.5 Hz, 3H), 2.80 (app d, J=6.5 Hz, 3H), 2.34 (s, 3H), 2.13-2.08 (m, 1H), 1.93-1.86 (m, 1H), 1.85-1.80 (m, 1H), 1.80-1.70 (m, 2H), 1.43 (s, 3H), 1.36 (app t, J=5.5 Hz, 3H), 1.30-1.08 (m, 5H).

The compounds in the following table were prepared by using methods similar to those described above in Example 4.

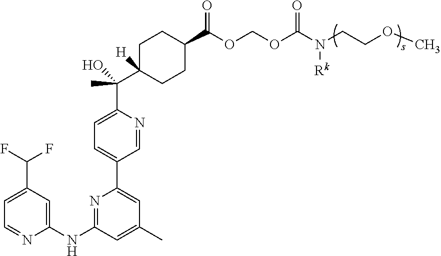

| | | | | [M + H]+ | [M + H]+ | |
|---|---|---|---|---|---|---|
| Ex. No. | R^{k} | s | Name | Calc'd | Obsv'd | Form(s) |
| 4-2 | —CH₃ | 1 | {[(2- | 628 | 628 | Free |
| | | | methoxyethyl)(methyl)carbamoyl]oxy}methyl | | | Base |
| | | | trans-4-[(1R)-1-(6-{[4- | | | |
| | | | (difluoromethyl)pyridin-2-yl]amino}- | | | |
| | | | 4-methyl-2,3′-bipyridin-6′-yl)-1- | | | |
| | | | hydroxyethyl]cyclohexanecarboxylate | | | |
| 4-3 | —H | 2 | 3-oxo-2,7,10-trioxa-4-azaundec-1-yl | 658 | 658 | Free |
| | | | trans-4-[(1R)-1-(6-{[4- | | | Base |
| | | | (difluoromethyl)pyridin-2-yl]amino}- | | | |
| | | | 4-methyl-2,3′-bipyridin-6′-yl)-1- | | | |
| | | | hydroxyethyl]cyclohexanecarboxylate | | | |

### Example 5

### Preparation of Compounds Using the General Method Illustrated in Scheme 7

### Example 5-1

### 4-{[({trans-4-[(1R)-1-(6-{[4-(Difluoromethyl)pyridin-2-yl]amino}-4-methyl-2,3′-bipyridin-6′-yl)-1-hydroxyethyl]cyclohexyl}carbonyl)oxy]methyl}-4-methylmorpholin-4-ium

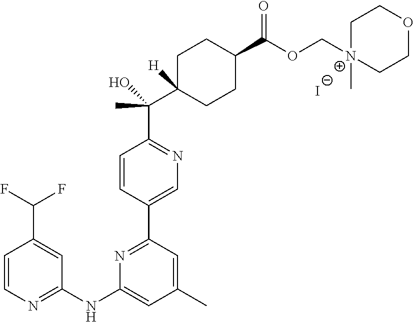 [SMILES: Cc1cc(Nc2cc(C(F)F)ccn2)nc(-c2ccc([C@](C)(O)[C@H]3CC[C@H](C(=O)OC[N+]4(C)CCOCC4)CC3)nc2)c1 ; [I-]]

To a suspension of sodium iodide (212 mg, 1.41 mmol) and chloromethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (750 mg, 1.41 mmol) in acetonitrile (15 mL) was added 4-methylmorpholine (0.155 mL, 1.41 mmol) and the reaction mixture was heated to 80° C. for 90 minutes. After cooling to room temperature, the reaction mixture was concentrated under reduced pressure and then lyophilized from methanol and water. The crude solid was purified using reverse phase HPLC (acetonitrile/water with formic acid as a modifier) to provide 4-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-4-methylmorpholin-4-ium iodide. ESI calc'd. for C₃₂H₄₀F₂N₅O₄ [M]+596. found 596. ¹H NMR (500 MHz, DMSO_{d-6}) δ 10.01 (s, 1H), 9.14 (d, J=2.0 Hz, 1H), 8.38 (m, 3H), 7.69 (d, J=8.3 Hz, 1H), 7.42 (s, 1H), 7.31 (s, 1H), 7.10 (t, J=56 Hz, 1H), 7.03 (d, J=5.2 Hz, 1H), 5.68 (s, 2H), 5.11 (br s, 1H), 3.90 (m, 4H), 3.46 (m, 2H), 3.39 (m, 2H), 3.14 (s, 3H), 2.41-2.37 (m, 1H), 2.34 (s, 3H), 2.04 (d, J=11.7 Hz, 1H), 1.89 (m, 2H), 1.77 (m, 1H), 1.45 (s, 3H), 1.45-1.35 (m, 1H), 1.30-1.10 (m, 4H).

The compounds in the following table were prepared by using methods similar to those described above in Example 5.

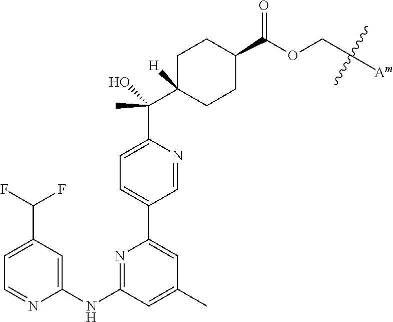

| Ex. | | | [M+] | [M+] | |
|---|---|---|---|---|---|
| No. | A^{m} | Name | Calc′d | Obsv′d | Form(s) |
| 5-2 | 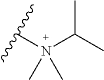 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-dimethylpropan-2- aminium | 582.3 | 582.5 | Iodide Salt |
| 5-3 | 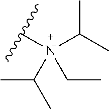 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N-ethyl-N-(1- methylethyl)propan-2-aminium | 624.4 | 624.7 | Iodide Salt |
| 5-4 | 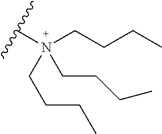 | N,N-dibutyl-N-{[({trans-4-[(1R)-1-(6- {[4-(difluoromethyl)pyridin-2- yl]amino}-4-methyl-2,3′-bipyridin-6′- yl)-1-hydroxyethyl]cyclohexyl} carbonyl)oxy]methyl}butan-1-aminium | 680.4 | 680.8 | Iodide Salt |
| 5-5 | 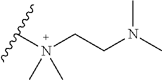 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-2-(dimethylamino)-N,N- dimethylethanaminium | 611.4 | 611.3 | Iodide Salt |
| 5-6 | 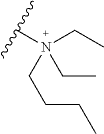 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-diethylbutan-1- aminium | 624.4 | 624.7 | Iodide Salt |
| 5-7 | 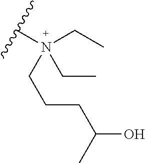 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-diethyl-4- hydroxypentan-1-aminium | 654.4 | 654.7 | Iodide Salt |
| 5-8 | 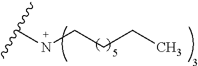 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-dioctyloctan-1- aminium | 848.6 | 848.5 | Iodide Salt |
| 5-9 | 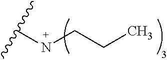 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-dipropylpropan-1- aminium | 638.4 | 638.8 | Iodide Salt |
| 5-10 | 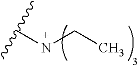 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-diethylethanaminium | 596.3 | 596.6 | Iodide Salt |
| 5-11 | 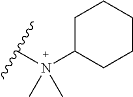 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N- dimethylcyclohexanaminium | 622.4 | 622.7 | Iodide Salt |
| 5-12 | 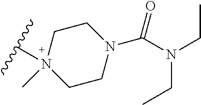 | 4-(diethylcarbamoyl)-1-{[({trans-4- [(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-1-methylpiperazin-1-ium | 694.4 | 694.8 | Iodide Salt |
| 5-13 | 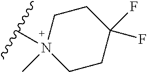 | 1-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-4,4-difluoro-1- methylpiperidinium | 630.3 | 630.7 | Iodide Salt |
| 5-14 | 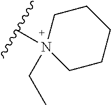 | 1-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-1-ethylpiperidinium | 608.3 | 608.6 | Iodide Salt |
| 5-15 | 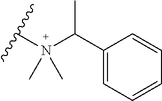 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-dimethyl-1- phenylethanaminium | 644.3 | 644.7 | Iodide Salt |
| 5-16 | 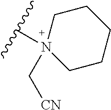 | 1-(cyanomethyl)-1-{[({trans-4-[(1R)- 1-(6-{[4-(difluoromethyl)pyridin-2- yl]amino}-4-methyl-2,3′-bipyridin-6′- yl)-1-hydroxyethyl]cyclohexyl}carbonyl) oxy]methyl}piperidinium | 619.3 | 619.6 | Iodide Salt |
| 5-17 | 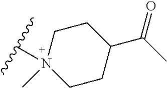 | 4-acetyl-1-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-1-methylpiperidinium | 636.3 | 636.6 | Iodide Salt |
| 5-18 | 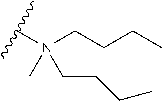 | N-butyl-N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N-methylbutan-1-aminium | 638.4 | 638.7 | Iodide Salt |
| 5-19 | 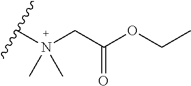 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-2-ethoxy-N,N-dimethyl-2- oxoethanaminium | 626.3 | 626.7 | Iodide Salt |
| 5-20 | 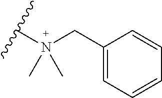 | N-benzyl[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]-N,N-dimethylmethanaminium | 630.3 | 630.6 | Iodide Salt |
| 5-21 | 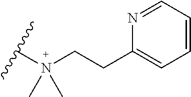 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-dimethyl-2-pyridin-2- ylethanaminium | 645.3 | 645.7 | Iodide Salt |
| 5-22 | 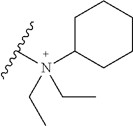 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N- diethylcyclohexanaminium | 650.4 | 650.7 | Iodide Salt |
| 5-23 | 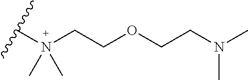 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-2-[2- (dimethylamino)ethoxy]-N,N- dimethylethanaminium | 655.4 | 655.7 | Iodide Salt |
| 5-24 | 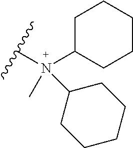 | N-cyclohexyl-N-{[({trans-4-[(1R)-1- (6-{[4-(difluoromethyl)pyridin-2- yl]amino}-4-methyl-2,3′-bipyridin-6′- yl)-1-hydroxyethyl]cyclohexyl}carbonyl) oxy]methyl}-N- methylcyclohexanaminium | 690.4 | 690.8 | Iodide Salt |
| 5-25 | 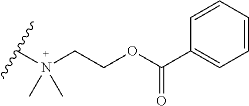 | N-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-N,N-dimethyl-2- [(phenylcarbonyl)oxy]ethanaminium | 688.3 | 688.7 | Iodide Salt |
| 5-26 | 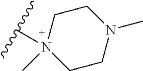 | 1-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl)oxy] methyl}-1,4-dimethylpiperazin-1-ium | 609.3 | 609.0 | Iodide Salt |
| 5-27 | 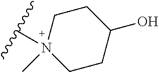 | 1-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-4-hydroxy-1- methylpiperidinium | 610.3 | 610.6 | Iodide Salt |
| 5-28 | 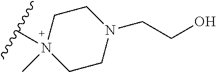 | 1-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-4-(2-hydroxyethyl)-1- methylpiperazin-1-ium | 639.4 | 639.7 | Iodide Salt |
| 5-29 | 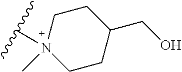 | 1-{[({trans-4-[(1R)-1-(6-{[4- (difluoromethyl)pyridin-2-yl]amino}- 4-methyl-2,3′-bipyridin-6′-yl)-1- hydroxyethyl]cyclohexyl}carbonyl) oxy]methyl}-4-(hydroxymethyl)-1- methylpiperidinium | 624.3 | 624.7 | Iodide Salt |

### Example 6

### Preparation of Compounds Using the General Method Illustrated in Scheme 8

### Example 6-1

### 1-{[({Trans-4-[(1R)-1-(6-{[4-(Difluoromethyl)pyridin-2-yl]amino}-4-methyl-2,3′-bipyridin-6′-yl)-1-hydroxyethyl]cyclohexyl}carbonyl)oxy]methyl}-3-methyl-1H-imidazol-3-ium

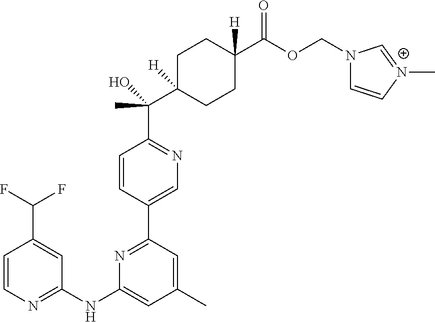 [SMILES: Cc1cc(Nc2cc(C(F)F)ccn2)nc(-c2ccc([C@](C)(O)[C@H]3CC[C@H](C(=O)OCn4cc[n+](C)c4)CC3)nc2)c1]

To a flask containing chloromethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (100 mg, 0.19 mmol), 1-methyl-1H-imidazole (15 μl, 0.19 mmol), and sodium iodide (28 mg, 0.19 mmol) was added acetonitrile (2.0 mL), and the mixture was heated to 80° C. overnight. The mixture was allowed to cool to room temperature and then diluted with ethyl acetate and water. The organic layer was separated, dried over magnesium sulfate, filtered and concentrated under reduced pressure. The crude residue was purified by reverse phase HPLC (10-100% acetonitrile/water with a 0.1% TFA modifier) to afford 1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-3-methyl-1H-imidazol-3-ium as the trifluoroacetate salt. MS ESI calc'd for C₃₁H₃₅F₂N₆O₃ [M]⁺ 577. found 577. ¹H NMR (500 MHz, DMSO-d₆) δ 10.09 (s, 1H), 9.28 (s, 1H), 9.14 (d, J=1.7 Hz, 1H), 8.50 (d, J=8.0 Hz, 1H), 8.39 (d, J=4.8 Hz, 1H), 8.28 (s, 1H), 7.81 (t, J=1.8 Hz, 1H), 7.77 (d, J=8.8 Hz, 1H), 7.71 (t, J=1.7 Hz, 1H), 7.47 (s, 1H), 7.34 (s, 1H), 7.10 (t, J=55.6 Hz, 1H), 7.05 (d, J=4.6 Hz, 1H), 6.07 (s, 2H), 5.31 (s, 1H), 3.85 (s, 3H), 2.35 (s, 3H), 2.26-2.18 (m, 1H), 2.01-1.90 (m, 1H), 1.90-1.68 (m, 3H), 1.47 (s, 3H), 1.32-1.07 (m, 5H).

The compounds in the following table were prepared by using methods similar to those described above in Example 6.

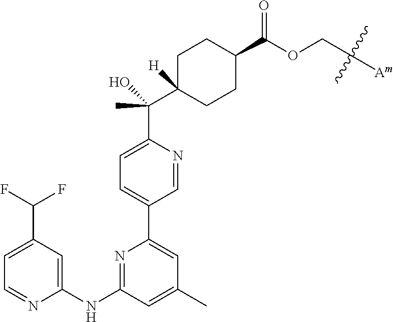

| Ex. | | | [M + H]⁺ | [M + H]⁺ | |
|---|---|---|---|---|---|
| No. | A^{m} | Name | Calc′d | Obsv′d | Form(s) |
| 6-2 | 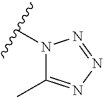 [SMILES: *n1nnnc1C] | (5-methyl-1H-tetrazol-1-yl)methyl trans- 4-[(1R)-1-(6-{[4-(difluoromethyl)pyridin- 2-yl]amino}-4-methyl-2,3′-bipyridin-6′- yl)-1-hydroxyethyl]cyclohexanecarboxylate | 579 | 579 | Free Base |
| 6-3 | 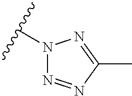 [SMILES: *n1nnc(C)n1] | (5-methyl-2H-tetrazol-2-yl)methyl trans- 4-[(1R)-1-(6-{[4-(difluoromethyl)pyridin- 2-yl]amino}-4-methyl-2,3′-bipyridin-6′- yl)-1-hydroxyethyl]cyclohexanecarboxylate | 579 | 579 | Free Base |
| 6-4 | 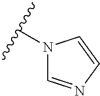 [SMILES: *n1ccnc1] | 1H-imidazol-1-ylmethyl trans-4-[(1R)-1- (6-{[4-(difluoromethyl)pyridin-2- yl]amino}-4-methyl-2,3′-bipyridin-6′-yl)- 1-hydroxyethyl]cyclohexanecarboxylate | 563 | 563 | TFA Salt |

### Example 7

### Preparation of Compounds Using the General Method Illustrated in Scheme 9

### Example 7-1a and 7-1b

### 2-[(R)-methylsulfinyl]ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate and 2-[(S)-methylsulfinyl]ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

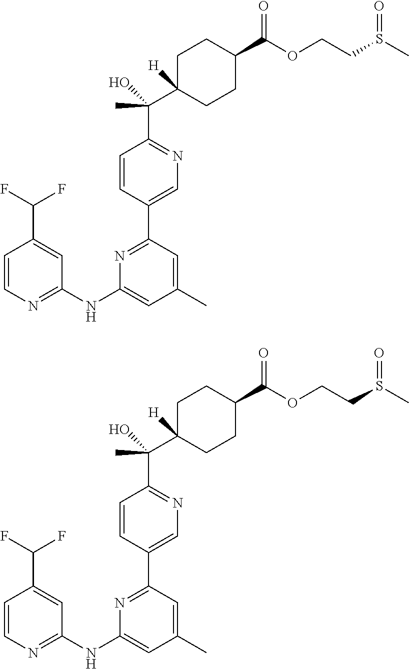 [SMILES: Cc1cc(Nc2cc(C(F)F)ccn2)nc(-c2ccc([C@](C)(O)[C@H]3CC[C@H](C(=O)OCC[S@@](C)=O)CC3)nc2)c1 ; Cc1cc(Nc2cc(C(F)F)ccn2)nc(-c2ccc([C@](C)(O)[C@H]3CC[C@H](C(=O)OCC[S@](C)=O)CC3)nc2)c1]

To a mixture of 2-(methylsulfanyl)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (75 mg, 0.14 mmol) in DCM (1.4 mL) at 0° C. was added m-CPBA (28 mg, 0.12 mmol), and the mixture was stirred at 0° C. for 30 minutes. The mixture was diluted with ethyl acetate and water and allowed to warm to room temperature. The organic layer was separated, dried over magnesium sulfate, filtered and concentrated under reduced pressure. The residue was purified by chromatography on silica gel. The diastereomers were separated by chiral SFC purification (Thar 80 preparative SFC, Chiralpak AS-H, 21×250 mm, 65% MeOH/CO₂ with a 0.25% dimethylethylamine modifier and a 12 minute run time) to yield 2-[(R)-methylsulfinyl]ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate and 2-[(S)-methylsulfinyl]ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate.

### Example 7-1a

Characterization data for the faster eluting enantiomer (Rₜ=5.79 min): MS ESI calc'd for C₂₉H₃₅F₂N₄O₄S [M+H]⁺ 573. found 573. ¹H NMR (500 MHz, DMSO-d₆) δ 10.00 (s, 1H), 9.13 (d, J=1.9 Hz, 1H), 8.40-8.35 (m, 2H), 8.32 (s, 1H), 7.68 (d, J=8.4 Hz, 1H), 7.42 (s, 1H), 7.31 (d, J=13.5 Hz, 1H), 7.10 (t, J=55.6 Hz, 1H), 7.02 (d, J=5.0 Hz, 1H), 5.07 (s, 1H), 4.41-4.30 (m, 1H), 4.31-4.16 (m, 1H), 3.12-3.02 (m, 1H), 2.97-2.82 (m, 1H), 2.55 (s, 3H), 2.34 (s, 3H), 2.18-2.08 (m, 1H), 1.99-1.89 (m, 1H), 1.88-1.77 (m, 2H), 1.77-1.67 (m, 1H), 1.43 (s, 3H), 1.34-1.07 (m, 5H).

### Example 7-1b

Characterization data for the slower eluting enantiomer (Rₜ=9.67 min): MS ESI calc'd for C₂₉H₃₅F₂N₄O₄S [M+H]⁺ 573. found 573. ¹H NMR (500 MHz, DMSO-d₆) δ 10.00 (s, 1H), 9.13 (d, J=1.9 Hz, 1H), 8.39-8.35 (m, 2H), 8.32 (s, 1H), 7.67 (t, J=9.0 Hz, 1H), 7.42 (s, 1H), 7.31 (d, J=13.8 Hz, 1H), 7.09 (t, J=55.6 Hz, 1H), 7.02 (d, J=5.2 Hz, 1H), 5.07 (s, 1H), 4.39-4.30 (m, 1H), 4.30-4.20 (m, 1H), 3.07 (ddd, J=4.9, 8.9, 13.7 Hz, 1H), 2.90 (dt, J=4.7, 13.6 Hz, 1H), 2.55 (s, 3H), 2.34 (s, 3H), 2.17-2.08 (m, 1H), 1.99-1.89 (m, 1H), 1.87-1.77 (m, 2H), 1.77-1.69 (m, 1H), 1.43 (s, 3H), 1.35-1.08 (m, 5H).

### Example 7-2

### ({[3-(Methylsulfonyl)propoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

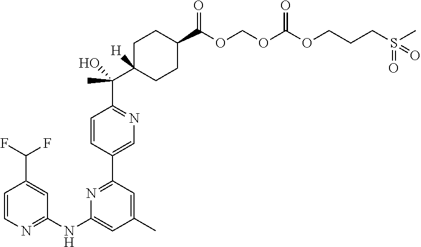 [SMILES: Cc1cc(Nc2cc(C(F)F)ccn2)nc(-c2ccc([C@](C)(O)[C@H]3CC[C@H](C(=O)OCOC(=O)OCCCS(C)(=O)=O)CC3)nc2)c1]

To a mixture of ({[3-(methylsulfanyl)propoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (2.23 g, 3.46 mmol) in DCM (35 mL) at 0° C. was added m-CPBA (1.51 g, 6.57 mmol), and the mixture was stirred for 30 minutes. The mixture was diluted with ethyl acetate and water. The organic layer was separated, dried over magnesium sulfate, filtered and concentrated. The crude residue was purified by chromatography on silica gel (0-100% ethyl acetate/hexanes) to afford ({[3-(methylsulfonyl)propoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd for C₃₂H₃₉F₂N₄O₈S [M+H]⁺ 677. found 677. ¹H NMR (500 MHz, DMSO-d₆) δ 10.00 (s, 1H), 9.13 (d, J=2.0 Hz, 1H), 8.39-8.35 (m, 2H), 8.32 (s, 1H), 7.68 (d, J=8.3 Hz, 1H), 7.42 (s, 1H), 7.33 (s, 1H), 7.15 (d, J=55.6 Hz, 1H), 7.02 (d, J=5.0 Hz, 1H), 5.65 (s, 2H), 5.07 (s, 1H), 4.20 (t, J=6.4 Hz, 2H), 3.20-3.11 (m, 2H), 2.96 (s, 3H), 2.34 (s, 3H), 2.23-2.15 (m, 1H), 2.07-1.90 (m, 4H), 1.88-1.69 (m, 2H), 1.43 (s, 3H), 1.25-1.08 (m, 5H).

The compounds in the following table were prepared by using methods similar to those described above in Example 7.

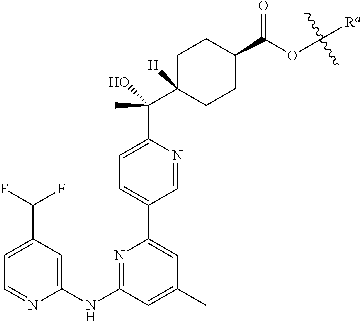

| Ex. | | | [M + H]⁺ | [M + H]⁺ | |
|---|---|---|---|---|---|
| No. | R^{a} | Name | Calc′d | Obsv′d | Form(s) |
| 7-3 | 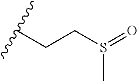 [SMILES: *CCS(C)=O] | 2-(methylsulfinyl)ethyl trans-4-[(1R)-1-(6-{[4- (difluoromethyl)pyridin-2- yl]amino}-4-methyl-2,3′- bipyridin-6′-yl)-1- hydroxyethyl]cyclohexane- carboxylate | 573 | 573 | Free Base |
| 7-4 | 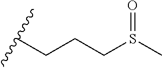 [SMILES: *CCCS(C)=O] | 3-(methylsulfinyl)propyl trans-4-[(1R)-1-(6-{[4- (difluoromethyl)pyridin-2- yl]amino}-4-methyl-2,3′- bipyridin-6′-yl)-1- hydroxyethyl]cyclohexane carboxylate | 587 | 587 | Free Base |
| 7-5 | 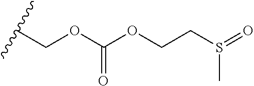 [SMILES: *COC(=O)OCCS(C)=O] | ({[2-(methylsulfinyl)ethoxy] carbonyl}oxy)methyl trans-4- [(1R)-1-(6-{[4- (difluoromethyl)pyridin-2- yl]amino}-4-methyl-2,3′- bipyridin-6′-yl)-1- hydroxyethyl]cyclohexane- carboxylate | 647 | 647 | Free Base |
| 7-6 | 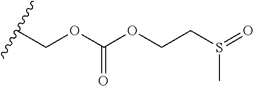 [SMILES: *COC(=O)OCCS(C)=O] | ({[2-(methylsulfinyl)ethoxy] carbonyl}oxy)methyl trans-4- [(1R)-1-(6-{[4- (difluoromethyl)pyridin-2- yl]amino}-4-methyl-2,3′- bipyridin-6′-yl)-1- hydroxyethyl]cyclohexane- carboxylate | 647 | 647 | Free Base |
| 7-7 | 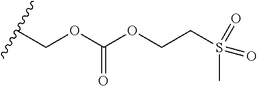 [SMILES: *COC(=O)OCCS(C)(=O)=O] | ({[2-(methylsulfonyl)ethoxy] carbonyl}oxy)methyl trans-4- [(1R)-1-(6-{[4- (difluoromethyl)pyridin-2- yl]amino}-4-methyl-2,3′- bipyridin-6′-yl)-1- hydroxyethyl]cyclohexane- carboxylate | 663 | 663 | Free Base |

### Example 8

### Preparation of Compounds Using the General Methods Illustrated in Scheme

### Example 8-1

### 17,17-Dihydroxy-17-oxido-3-oxo-2,4,7,10,13,16-hexaoxa-17λ⁵-phosphaheptadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

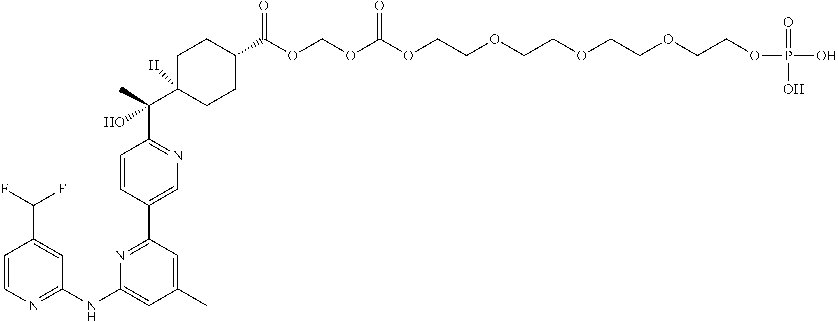 [SMILES: Cc1cc(Nc2cc(C(F)F)ccn2)nc(-c2ccc([C@](C)(O)[C@H]3CC[C@H](C(=O)OCOC(=O)OCCOCCOCCOCCOP(=O)(O)O)CC3)nc2)c1]

To a solution of 15-hydroxy-3-oxo-2,4,7,10,13-pentaoxapentadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (270 mg, 0.37 mmol) in DCM (3 mL) at 0° C. was added Hunig's base (0.64 mL, 3.7 mmol) followed by the dropwise addition of diphosphoryl chloride (0.10 mL, 0.74 mmol). The reaction mixture was stirred at 0° C. for 10 minutes. The reaction mixture was then concentrated under reduced pressure. To this residue was added a 1:2 mixture of water/acetonitrile (16 mL). The resulting solution was purified directly by reverse phase HPLC (5-60% acetonitrile/water with 0.1% TFA modifier). Fractions containing product were frozen and lyophilized to afford 17,17-dihydroxy-17-oxido-3-oxo-2,4,7,10,13,16-hexaoxa-17λ⁵-phosphaheptadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. The product was isolated as a TFA salt. MS ESI calc'd. for C₃₆H₄₈F₂N₄O₁₃P [M+H]⁺ 813. found 813. ¹H NMR (500 MHz, DMSO-d₆) δ 10.19 (s, 1H), 9.14 (d, J=2.0 Hz, 1H), 8.66 (s, 1H), 8.41 (d, J=5.5 Hz, 1H), 8.18 (s, 1H), 7.90 (d, J=8.0 Hz, 1H), 7.54 (s, 1H), 7.42 (s, 1H), 7.10 (t, J=55.5 Hz, 1H), 7.07 (d, J=5.5 Hz, 1H), 5.65 (s, 2H), 4.23-4.19 (m, 2H), 3.90-3.85 (m, 2H), 3.61-3.57 (m, 2H), 3.55-3.52 (m, 2H), 3.51-3.45 (m, 8H), 2.36 (s, 3H), 2.25-2.18 (m, 1H), 1.98-1.92 (m, 1H), 1.90-1.84 (m, 1H), 1.84-1.73 (m, 2H), 1.51 (s, 3H), 1.34-1.24 (m, 1H), 1.23-1.10 (m, 4H).

The compounds in the following table were prepared by using methods similar to those described above in Example 8.

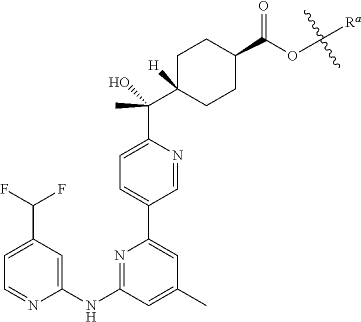

| Ex. | | | [M + H]⁺ | [M + H]⁺ | |
|---|---|---|---|---|---|
| No. | R^{a} | Name | Calc′d | Obsv′d | Form(s) |
| 8-2 | 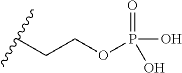 [SMILES: *CCOP(=O)(O)O] | 2-(phosphonooxy)ethyl trans- 4-[(1R)-1-(6-{[4- (difluoromethyl)pyridin-2- yl]amino}-4-methyl-2,3′- bipyridin-6′-yl)-1- hydroxyethyl]cyclohexane- carboxylate | 607 | 607 | TFA Salt |

### Example 9

### Alkylation of Amino-Substituted R^{CH} Group-Containing Compounds

### Example 9-1

### Trans-4-(Dimethylamino)cyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate

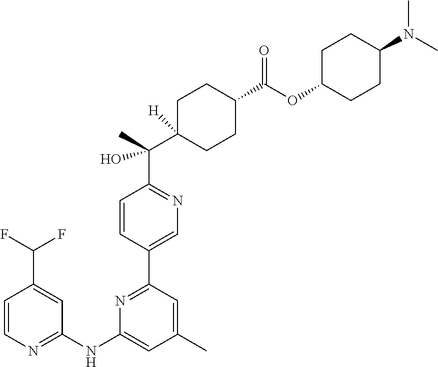 [SMILES: Cc1cc(Nc2cc(C(F)F)ccn2)nc(-c2ccc([C@](C)(O)[C@H]3CC[C@H](C(=O)O[C@H]4CC[C@H](N(C)C)CC4)CC3)nc2)c1]

To a solution of trans-4-aminocyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate tris(trifluoroacetate) (salt) (250 mg, 0.271 mmol) in methanol (10 mL) at 20° C. was added formaldehyde (30% solution in water, 0.20 mL, 2.7 mmol) and triethylamine (0.15 mL, 1.09 mmol) followed by sodium cyanoborohydride (170 mg, 2.7 mmol). The reaction mixture was stirred at 20° C. for 4 hours. The reaction mixture was then diluted with ethyl acetate (100 mL) and washed with saturated aqueous sodium bicarbonate solution (2×20 mL), water (20 mL), and brine (20 mL). The organic layer was separated, dried over magnesium sulfate, filtered, and concentrated under reduced pressure. The residue was azeotroped with acetonitrile (3×10 mL). The residue was then diluted with acetonitrile (2 mL) and water (6 mL), and the resulting suspension was frozen and lyophilized to afford trans-4-(dimethylamino)cyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₃₄H₄₄F₂N₅O₃ [M+H]⁺ 608. found 608. ¹H NMR (500 MHz, DMSO-d₆) δ 10.00 (s, 1H), 9.13 (d, J=2.0 Hz, 1H), 8.39-8.35 (m, 2H), 8.33 (s, 1H), 7.67 (d, J=9.0 Hz, 1H), 7.42 (s, 1H), 7.32 (s, 1H), 7.09 (t, J=55.5 Hz, 1H), 7.02 (d, J=5.5 Hz, 1H), 5.05 (s, 1H), 4.52-4.46 (m, 1H), 2.34 (s, 3H), 2.12 (s, 6H), 2.08-2.00 (m, 1H), 1.92-1.69 (m, 8H), 1.43 (s, 3H), 1.30-1.08 (m, 10H).

### Example 10

### Preparation of Methylene-Linked Phosphate Esters

### Example 10-1

### (Phosphonooxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate OOH

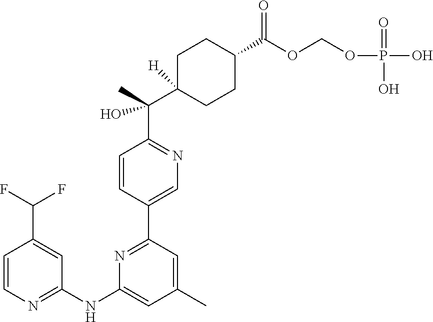 [SMILES: Cc1cc(Nc2cc(C(F)F)ccn2)nc(-c2ccc([C@](C)(O)[C@H]3CC[C@H](C(=O)OCOP(=O)(O)O)CC3)nc2)c1]

Step 1:

To a mixture of trans-4-[(1R)-1-(6-{[4-(difluoromethyl)pyridin-2-yl]amino}-4-methyl-2,3′-bipyridin-6′-yl)-1-hydroxyethyl]cyclohexanecarboxylic acid (1.00 g, 2.07 mmol), potassium carbonate (1.43 g, 10.4 mmol), and sodium iodide (0.062 g, 0.41 mmol) in DMF (10 mL) was added dibenzyl(chloromethyl)phosphate (1.35 g, 4.1 mmol) at 20° C. The reaction mixture was stirred at 20° C. for 16 hours. The reaction mixture was then diluted with ethyl acetate (250 mL) and washed with water (3×50 mL) followed by brine (1×50 mL). The organic layer was separated, dried over magnesium sulfate, filtered, and concentrated under reduced pressure. The crude product residue was purified by silica gel chromatography (0-100% ethyl acetate/hexanes) to afford {[bis(benzyloxy)phosphoryl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. MS ESI calc'd. for C₄₁H₄₄F₂N₄O₇P [M+H]⁺ 773. found 773.

Step 2:

A mixture of {[bis(benzyloxy)phosphoryl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate (1.15 g, 1.49 mmol) and palladium on carbon (0.16 g, 0.15 mmol) was degassed with argon for 5 minutes. To the degassed mixture was added methanol (30 mL). The headspace of the flask was evacuated and then backfilled with hydrogen gas. The reaction mixture was stirred for 2 hours under a hydrogen atmosphere. The reaction mixture was then filtered through CELITE and the filter cake was washed with methanol. The filtrate was concentrated under reduced pressure. The residue was purified by reverse phase HPLC (10-50% acetonitrile/water with 0.1% TFA modifier). Fractions containing product were frozen and lyophilized to afford (phosphonooxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate. The product was isolated as a TFA salt. MS ESI calc'd. for C₂₇H₃₂F₂N₄O₇P [M+H]⁺ 593. found 593. ¹H NMR (500 MHz, DMSO-d₆) δ 10.30 (s, 1H), 9.16 (d, J=2.0 Hz, 1H), 8.76 (d, J=8.0 Hz, 1H), 8.41 (d, J=5.0 Hz, 1H), 8.13 (s, 1H), 7.98 (d, J=8.0 Hz, 1H), 7.58 (s, 1H), 7.44 (s, 1H), 7.10 (t, J=55.5 Hz, 1H), 7.08 (d, J=5.5 Hz, 1H), 5.41 (d, J=13.5 Hz, 2H), 2.37 (s, 3H), 2.22-2.14 (m, 1H), 2.01-1.95 (m, 1H), 1.92-1.74 (m, 3H), 1.55 (s, 3H), 1.36 (m, 1H), 1.24-1.10 (m, 4H).

### Biological Assay

While not being bound by any specific theory, it is anticipated that the compounds of Formula (I) will cleave to provide trans-4-[(1R)-(6-{[4-(difluoromethyl)pyridin-2-yl]amino}-4-methyl-2,3′-bipyridin-6′-yl)-1-hydroxyethyl]cyclohexanecarboxylic acid (PrepEx 1-5), and thereby inhibit Syk. Nevertheless, certain of the compounds of Formula (I) themselves also inhibit Syk in an in vitro assay. Syk inhibition of the compounds of Formula (I) may be determined using the following assay protocol:

Homogeneous Time-Resolved Fluorescence (HTRF) Assay for the Recombinant Human Syk Enzyme:

A recombinant GST-hSyk fusion protein was used to measure potency of compounds to inhibit human Syk activity. The recombinant human GST-Syk (Carna Biosciences #08-176) (5 pM final concentration) was incubated with various concentrations of the inhibitor diluted in DMSO (0.1% final concentration) for 10 minutes at room temperature in 15 mM Tris-HCl (pH 7.5), 0.01% tween 20, 2 mM DTT in 384 well plate format. To initiate the reaction the biotinylated substrate peptide (250 nM final concentration) that contains the phosphorylation site for Syk was added with magnesium (5 mM final concentration) and ATP (25 μM final concentration). Final volume of the reaction was 10 μL. Phosphorylation of the peptide was allowed to proceed for 45′ at room temperature. To quench the reaction and detect the phosphorylated product, 2 nM of a Europium-anti-phosphotyrosine antibody (Perkin Elmer #AD0161) and 70 nM SA-APC (Perkin-Elmer #CR130-100) were added together in 15 mM Tris pH 7.5, 40 mM EDTA, 0.01% tween 20. Final volume of the quenching solution was 10 μL. The resulting HTRF signal was measured after 30 minutes on a EnVision (Perkin-Elmer) reader using a time-resolved fluorescence protocol. IC₅₀ was determined by fitting the observed relationship between compound concentration (10-dose titration, 10 μM to 0.508 nM) and HTRF signal with a 4-parameter logistic equation. Representative compounds of the invention were tested in the assay, and their IC₅₀ values are set forth in the table below. Trans-4-[(1R)-(6-{[4-(difluoromethyl)pyridin-2-yl]amino}-4-methyl-2,3′-bipyridin-6′-yl)-1-hydroxyethyl]cyclohexanecarboxylic acid (PrepEx 1-5) provides an IC₅₀ of <0.5 nM, when tested in the assay.

| Ex. No. | IC₅₀ (nM) |
|---|---|
| 1-1 | 8.1 |
| 1-2 | 4.2 |
| 1-3 | 2.5 |
| 1-4 | 0.8 |
| 1-5 | 4.4 |
| 1-6 | 9.7 |
| 1-7 | 18.2 |
| 1-8 | 4.0 |
| 1-9 | 27.7 |
| 1-10 | 1.7 |
| 1-11 | 2.1 |
| 1-12 | 10.0 |
| 1-13 | 4.4 |
| 1-14 | 27.6 |
| 1-15 | 18.5 |
| 1-16 | 2.3 |
| 1-17 | 4.1 |
| 1-18 | 2.7 |
| 1-19 | 4.4 |
| 1-20 | 34.1 |
| 1-21 | 2.3 |
| 1-22 | 1.8 |
| 1-23 | 1.6 |
| 1-24 | 3.5 |
| 1-25 | <0.5 |
| 1-26 | 4.1 |
| 1-27 | 1.8 |
| 1-28 | 7.2 |
| 1-29 | 7.3 |
| 2-1 | 19.9 |
| 2-2 | 173.8 |
| 2-3 | 6.2 |
| 2-4 | 5.3 |
| 2-5 | 7.5 |
| 2-6 | <0.5 |
| 2-7 | 78.0 |
| 2-8 | 22.8 |
| 2-9 | 27.2 |
| 2-10 | <0.5 |
| 2-11 | 5.2 |
| 2-12 | 1.5 |
| 2-13 | 34.6 |
| 2-14 | 30.4 |
| 2-15 | 32.9 |
| 2-16 | 48.4 |
| 2-17 | <0.5 |
| 3-1 | 27.0 |
| 3-2 | 1.9 |
| 3-2a | 19.1 |
| 3-2b | 22.4 |
| 3-3 | 2.3 |
| 3-4 | 1.8 |
| 3-5 | <0.5 |
| 3-6 | 4.1 |
| 3-7 | 12.7 |
| 3-8 | 8.3 |
| 3-9 | 4.8 |
| 3-29 | 1.2 |
| 4-1 | 20.7 |
| 4-1a | 24.7 |
| 4-1b | 12.9 |
| 4-2 | 9.1 |
| 4-3 | 2.4 |
| 5-1 | 3.6 |
| 5-2 | 0.8 |
| 5-3 | 1.9 |
| 5-4 | 1.1 |
| 5-5 | 1.3 |
| 5-6 | 1.1 |
| 5-7 | 1.3 |
| 5-8 | 2.5 |
| 5-9 | 1.0 |
| 5-10 | 1.4 |
| 5-11 | 0.9 |
| 5-12 | <0.5 |
| 5-13 | 1.3 |
| 5-14 | 2.3 |
| 5-15 | <0.5 |
| 5-16 | 0.6 |
| 5-17 | 1.6 |
| 5-18 | 1.4 |
| 5-19 | 0.8 |
| 5-20 | 0.8 |
| 5-21 | <0.5 |
| 5-22 | 0.9 |
| 5-23 | 1.1 |
| 5-24 | 1.0 |
| 5-25 | 0.7 |
| 5-26 | 2.0 |
| 5-27 | 1.5 |
| 5-28 | 2.9 |
| 5-29 | <0.5 |
| 6-1 | 0.8 |
| 7-1a | 4.2 |
| 7-1b | 3.3 |
| 7-2 | 7.5 |
| 7-3 | 3.3 |
| 7-4 | 4.3 |
| 7-5 | 0.6 |
| 7-6 | 1.0 |
| 7-7 | 4.5 |
| 8-1 | <0.5 |
| 8-2 | <0.5 |
| 9-1 | 3.2 |
| 10-1 | <0.5 |

The suitability of the compounds of Formula (I) as prodrugs of Syk inhibitors can be tested as described below.

### Solubility in Biorelevant Fasted State Simulated Intestinal Fluid (FaSSIF):

A 10 mM DMSO stock solution of compound to be tested is used. A 100 μM standard solution is generated by diluting 2.5 μL of the 10 mM stock solution with 247.5 μL of diluent (10% DMSO/10% MeCN/80% MeOH, v/v/v). A solubility solution is generated by diluting 5 μL of the 10 mM stock solution with 245 μL of FaSSIF (maximum solubility approximately 200 μM). The solubility solution is equilibrated overnight at 25° C. with gentle shaking. The equilibrated solubility solution is filtered by centrifugation (3 minutes at 4000 rpm) using a 0.45 m filter with a polypropylene membrane. The standard solution and the filtered equilibrated solubility solution are analyzed by HPLC/DAD. Representative compounds of the invention were tested in the assay, and their solubilities are set forth in the table below. The solubility value is calculated by the following equation:

Solubility=(Peak area of sample/Peak area of standard)(Standard concentration)

| Ex. No. | Solubility (μM) |
|---|---|
| Prep Ex 1-5 | <3 |
| 1-2 | 114 |
| 1-5 | 164 |
| 1-9 | 43 |
| 1-11 | 57 |
| 1-12 | 76 |
| 1-13 | 140 |
| 1-14 | 109 |
| 1-15 | 126 |
| 1-16 | 165 |
| 1-17 | 136 |
| 1-18 | 109 |
| 1-19 | 98 |
| 1-20 | 47 |
| 1-21 | 167 |
| 1-22 | 183 |
| 1-23 | 131 |
| 1-24 | 141 |
| 1-25 | 73 |
| 1-26 | 109 |
| 1-27 | 50 |
| 2-1 | 167 |
| 2-7 | 119 |
| 2-11 | 149 |
| 2-16 | 48 |
| 3-1 | 147 |
| 3-4 | 59 |
| 3-6 | 89 |
| 3-9 | 160 |
| 3-29 | 41 |
| 5-1 | 200 |
| 5-2 | 97 |
| 5-3 | 175 |
| 5-5 | 53 |
| 5-6 | 152 |
| 5-7 | 200 |
| 5-9 | 137 |
| 5-10 | 138 |
| 5-11 | 200 |
| 5-12 | 55 |
| 5-14 | 200 |
| 5-16 | 69 |
| 5-17 | 45 |
| 5-20 | 86 |
| 5-23 | 98 |
| 5-25 | 155 |
| 5-26 | 68 |
| 5-27 | 164 |
| 5-29 | 176 |
| 6-1 | 140 |
| 6-4 | 156 |
| 7-4 | 141 |
| 7-6 | 156 |
| 8-1 | 154 |
| 8-2 | 161 |
| 9-1 | 162 |
| 4-1a | 40 |
| 7-1a | 56 |
| 7-1b | 52 |

### Conversion of Prodrug to Parent in In Vitro Biorelevant Gastrointestinal Tract Media:

The likely stability of prodrugs in the gastrointestinal tract can be estimated by incubating a prodrug in simulated gastric and intestinal fluids; compounds were measured for stability in simulated gastric fluid (SGF), fasted state simulated intestinal fluid (FaSSIF), and fasted state simulated intestinal fluid plus pancreatin (FaSSIF+pancreatin). Prodrug stock solutions were made by diluting 2 mg of compound in 200 μL DMSO. A 10 mg/mL Pancreatin in FaSSIF stock solution was made by dissolving 10 mg Pancreatin in 1 mL FaSSIF. Samples were prepared by diluting 10 μL of compound stock solution into 990 μL of biorelevant media (990 μL SGF, 990 μL FaSSIF, or 940 μL FaSSIF plus 50 μL Pancreatin stock) in a 2 mL HPLC vial and vortexing the mixture for a few seconds. The samples were incubated at 37° C. and aliquots were removed for up to 24 hours and injected directly on an HPLC to determine the conversion of prodrug to active drug. In the case of compound precipitation upon sample preparation, an individual sample was prepared for each desired time point and the entire sample was diluted with a known volume of acetonitrile/water (50/50) to ensure compound dissolution before HPLC analysis. Representative compounds of the invention were tested in the assay, and their conversons are set forth in the table below.

| | Conversion | Conversion | Conversion |
|---|---|---|---|
| | in SGF, | in FaSSIF, | in FaSSIF + |
| Ex. No. | time | time | Pancreatin, time |
| 1-21 | 0%, 5 h | 0%, 5 h | 100%, 3 h |
| 1-22 | 0%, 5 h | 0%, 5 h | 100%, 3 h |
| 1-23 | 0%, 5 h | 0%, 5 h | 100%, 3 h |
| 1-29 | 0%, 5 h | 0%, 5 h | 44%, 24 h |
| 2-2 | 0%, 5 h | 0%, 5 h | 100%, 3 h |
| 2-4 | 0%, 5 h | 5%, 24 h | 100%, 3 h |
| 2-6 | 0%, 5 h | 0%, 5 h | 95%, 3 h |
| 2-7 | 0%, 5 h | 0%, 5 h | 20%, 3 h |
| 2-9 | 2%, 3 h | 0%, 5 h | 65%, 3 h |
| 3-1 | 0%, 5 h | 100%, 3 h | 100%, 3 h |
| 5-1 | 0%, 5 h | 12%, 3 h | 45%, 3 h |
| 8-2 | 0%, 5 h | 0%, 5 h | 20%, 3 h |
| 10-1 | 5%, 5 h | 20%, 5 h | 50%, 5 h |

### Conversion of Prodrug to Parent in Intestine Homogenate:

The likely stability of prodrugs in the intestine can be estimated by incubating a prodrug with intestine homogenate. Incubations of prodrugs (1 μM) with intestine S9 (1 mg protein/mL) were carried out at 37° C. in a phosphate buffer, pH 7.4. Control incubations contained BSA (1.1 mg/mL) instead of intestine S9 microsomes. Aliquots were removed at 0, 5, 15, 30, 60 and 120 min, treated with 4 volumes of acetonitrile containing 2% formic acid and an internal standard, and centrifuged. The supernatants were analyzed by LC-MS/MS for prodrug disappearance and appearance of active drug. The half-life of the prodrug was calculated from the % prodrug remaining at different time points calculated from on the peak area ratio relative to t=0. The amount of active drug generated at the different time points was determined using a standard curve. Representative compounds of the invention were tested in the assay, and the results are set forth in the table below.

| | Prodrug concentration | Parent acid concentration |
|---|---|---|
| Ex. No. | at 2 h (μM) | at 2 h (μM) |
| 2-2 | 0 | 0.937 |
| 2-17 | 0.371 | 0.606 |
| 4-1 | 0.539 | 0.335 |
| 5-1 | 0.321 | 0.239 |
| 6-1 | 0.844 | 0.263 |
| 7-1a | 0.385 | 0.523 |
| 7-2 | 0.092 | 0.822 |
| 8-1 | 0 | 0.757 |
| 8-2 | 0.249 | 0.368 |

### Conversion of Prodrug to Parent in Liver Homogenate:

The likely stability of prodrugs in the liver can be estimated by incubating a prodrug with liver homogenate. Incubations of prodrugs (1 μM) with liver S9 (1 mg protein/mL) were carried out at 37° C. in a phosphate buffer, pH 7.4. Control incubations contained BSA (1.1 mg/mL) instead of liver S9 microsomes. Aliquots were removed at 0, 5, 15, 30, 60 and 120 min, treated with 4 volumes of acetonitrile containing 2% formic acid and an internal standard, and centrifuged. The supernatants were analyzed by LC-MS/MS for prodrug disappearance and appearance of active drug. The half-life of the prodrug was calculated from the % prodrug remaining at different time points calculated from on the peak area ratio relative to t=0. The amount of active drug generated at the different time points was determined using a standard curve. Representative compounds of the invention were tested in the assay, and the results are set forth in the table below.

| | Prodrug concentration | Parent acid concentration |
|---|---|---|
| Ex. No. | at 2 h (μM) | at 2 h (μM) |
| 2-2 | 0 | 0.744 |
| 2-17 | 0 | 0.959 |
| 4-1 | 0 | 0.816 |
| 5-1 | 0.314 | 0.269 |
| 6-1 | 0.622 | 0.585 |
| 7-1a | 0 | 0.598 |
| 7-2 | 0.12 | 0.875 |
| 8-1 | 0 | 0.902 |
| 8-2 | 0.434 | 0.279 |

### Blood Stability Assay:

The likely stability of prodrugs in the blood can be estimated by incubating a prodrug with blood. A prodrug stock solution was prepared at 10 mM in DMSO and working solutions (100 μM) containing the prodrug were prepared by diluting a suitable amount of the stock solution with acetonitrile/water (50/50). 5 μL of the working solution were added to 495 μL of pre-warmed blood (at 37° C.). After vortexing and mixing, 50 μL of the spiked blood sample were taken for the 0 time point and transferred to another vial containing 200 μL of quenching solution (labetalol, alprazolam and diclofenac in 100% acetonitrile) ensuring that the vial is lidded to prevent evaporation and kept on ice. While keeping the spiked blood sample at 37° C. at the following timepoints (0.25, 0.5, 1, 2 and 3 h) 5 additional aliquots (50 μL) were sampled and mixed with the quenching solution. At the end of the experiment the quenched samples were vortexed for 2 minutes and centrifuged for 10 minutes at 4000 rpm. The supernatant (100 μL) was then transferred and collected into a new 96-well plate and injected into the LC-MS/MS for analysis. Representative compounds of the invention were tested in the assay, and their half lives are set forth in the table below. For each test compound, the area ratios at each time point to the 0 hour area ratio were compared and converted to a percentage remaining. The percentage remaining were then plotted against time (h) and half life generated using the following equation t½=0.693/kel. Where kel is the elimination rate constant which is determined from the concentration versus time curve by linear regression at the terminal phase of the semi-logarithmic plot.

| Ex. No. | Human blood stability half life (min) |
|---|---|
| 1-8 | 22 |
| 2-2 | 7 |
| 2-14 | 199 |
| 3-4 | 14 |
| 3-29 | 29 |
| 4-2 | 16 |
| 5-1 | 15 |
| 6-1 | 24 |

### In Vivo Pharmacokinetics Assay:

The in vivo conversion of a prodrug to the parent drug can be examined by dosing an animal with the prodrug and measuring the amount of parent drug produced using a pharmacokinetics assay. A prodrug stock solution was prepared at 10 mM in DMSO and two different working solutions (100 μM) containing the analyte were prepared by diluting a suitable amount of the stock solution with acetonitrile/water (50/50) and further diluted for the calibration standards (STD, from 0.0001 to 10 μM) and six quality control samples (QC at 0.0005, 0.0025, 0.005, 0.75, 3.75 and 7.5 μM) preparation. Unknown samples obtained from dosed animals were prepared for analysis by means of a single step protein precipitation technique by adding 200 μL of IS (internal standard) crashing solvent (2 ²H and 1 ¹³C stable labeled compound) to 50 μL aliquots of individual subject samples. Samples were mixed by vortex for homogeneity and then subjected to centrifugation at 4000 rpm for 15 minutes. The supernatant (200 μL) was then transferred and collected into a new 96-well plate and injected into the LC-MS/MS for analysis. The chromatographic data were collected and integrated using Analyst 1.4.2 data analysis program. Peak area ratios of the analyte to IS were utilized for construction of the calibration curve. A weighting of 1/x² (least-squares linear regression analysis, where x is the concentration of a given standard) was used for curve fit. Concentrations in unknown samples were calculated from the best-fit equation (y=mx+b) where y is the peak area ratio. The regression equation for the calibration curve was also used to back-calculate the measured concentration at each quality control level, and the results were compared with the theoretical concentration to obtain the accuracy expressed as a percentage of the theoretical value.

Representative compounds of the invention were tested in the assay, and the results are set forth in the tables below. Pharmacokinetic parameters were calculated using established non-compartmental methods. The area under the plasma concentration versus time curve (AUC) was determined using the Watson software (version 7.3), with linear trapezoidal interpolation in the ascending slope and logarithmic trapezoidal interpolation in the descending slope. The portion of the AUC from the last measurable concentration to infinity was estimated from the equation, Ct/kel, where Ct represents the last measurable concentration and kel is the elimination rate constant. The latter was determined from the concentration versus time curve by linear regression at the terminal phase of the semi-logarithmic plot.

Rat data

| | Prodrug PO | | Parent produced |
|---|---|---|---|
| Ex. No. | Dose, mpk | # of rats | (AUC(0-∞), μM · h) |
| 2-2 | 1 | 4 | 4.93 |
| 5-1 | 1 | 4 | 5.71 |
| 8-2 | 1 | 4 | 9.75 |

| | Prodrug PO | | Parent produced |
|---|---|---|---|
| Ex. No. | Dose, mpk | # of dogs | (AUC(0-∞), μM · h) |
| 2-2 | 0.5 | 4 | 1.93 |
| 5-1 | 0.5 | 4 | 1.58 |
| 8-2 | 0.5 | 4 | 2.82 |

1. A compound of the Formula (I): 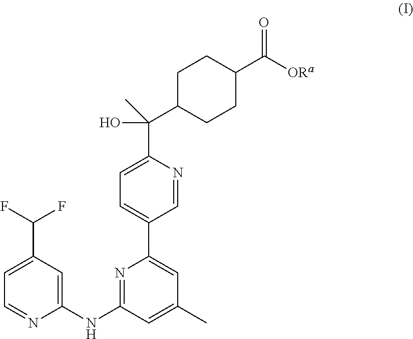
or a pharmaceutically acceptable salt thereof, wherein R^{a} is selected from the group consisting of:
A. C₁₋₃alkyl substituted by 1 to 3 moieties selected from the group consisting of —OH, —N(R^{e})₂, —N(C₁₋₄alkyl)₃, —C(O)N(R^{e})₂, —S—(C₁₋₄ alkyl), —S(O)—(C₁₋₄ alkyl), and —S(O)₂—(C₁₋₄ alkyl);
B. C₄₋₈alkyl, wherein said C₄₋₈alkyl is unsubstituted or substituted by 1-3 moieties selected from the group consisting of —OH, —N(R^{e})₂, —N(C₁₋₄alkyl)₃, —C(O)N(R^{e})₂, —S—(C₁₋₄ alkyl), —S(O)—(C₁₋₄ alkyl), and —S(O)₂—(C₁₋₄ alkyl);
C. a group of the formula -M-R^{CH}, wherein
1. M is a bond or —(CH₂)₁₋₂—;
2. R^{CH} is a cyclic moiety selected from the group consisting of:
a.) aryl or carbocyclyl;
b.) a 5- to 9-membered mono or bicyclic heterocyclyl containing 1 or 2 heteroatoms independently selected from the group consisting of N and O; and
c.) 5- to 6-membered heteroaryl containing 1 or 4 heteroatoms independently selected from the group consisting of N, O, and S;
wherein R^{CH} is unsubstituted or substituted with 1-4 moieties independently selected from the group consisting of halo, C₁₋₄alkyl, C₁₋₄alkoxy, —CN, —CO₂H, —OH, —N(R^{e})₂, and —N(C₁₋₄alkyl)₃;
D. a group of the formula 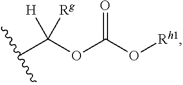
wherein
1. R^{g} is H or C₁₋₄alkyl; and
2. R^{h} is
a.) —Y¹—C^{A}; wherein
(i) Y¹ is a bond, —CH₂—, or —CH₂CH₂—O—CH₂—; and
(ii) C^{A} is a cyclic moiety selected from the group consisting of:
(I) C₃₋₆ cycloalkyl;
(II) phenyl;
(III) a 5- or 6-membered heterocyclyl containing 1 to 2 heteroatoms selected from the group consisting of N and O; and
(IV) a 5- or 6-membered heteroaryl containing 1 to 2 heteroatoms selected from the group consisting of N and 0;
wherein C^{A} is unsubstituted or substituted by 1 to 3 moieties independently selected from the group consisting of C₁₋₃alkyl, halo, or —N(R^{e})₂;
b.) C₂₋₆ alkyl, wherein said C₂₋₆alkyl of R^{h1} is unsubstituted or substituted by 1 to 3 moieties independently selected from the group consisting of —OH, C₁₋₃alkoxy, —CH₂—O—C₁₋₃alkyl, —N(R^{e})₂, —S—C₁₋₃alkyl, —S(O)—C₁₋₃alkyl, —S(O)₂—C₁₋₃alkyl, —P(O)(OH)₂, and —C(O)—N(R^{e})₂;
c.) a group of the formula 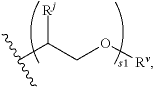
wherein
(i) R^{j} is H C₁₋₃alkyl, or —CH₂—O—C₁₋₃alkyl;
(ii) R^{v} is H, C₁₋₃alkyl, or —P(O)(OH)₂, and
(iii) the subscript s1 is 2, 3, 4, or 5;
E. a group of the formula 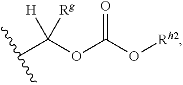
wherein
1. R^{g} is as set forth above;
2. R^{h2} is:
a.) —Y²—C^{B}; wherein
(i) Y² is a bond or a group 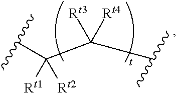
wherein
(I) R^{t1} and R^{t2} are independently H, C₁₋₃alkyl or —OH;
(II) each occurrence of R^{t3} and R^{t4} are independently H or C₁₋₃alkyl; and
(III) the subscript t is 0, 1, 2, 3, or 4;
(ii) C^{B} is a cyclic moiety selected from the group consisting of:
(I) C₄₋₆cycloalkyl, cholic acid, or chenodeoxycholic acid,
(II) phenyl,
(III) 5- or 6-membered heterocyclyl containing 1 to 2 heteroatoms selected from the group consisting of N, O, and S; and
(IV) 5- or 6-membered heteroaryl containing 1 to 2 heteroatoms selected from the group consisting of N, O, and S; and
wherein C^{B} is unsubstituted or substituted by 1 to 3 moieties independently selected from the group consisting of C₁₋₃alkyl, C₁₋₃alkoxy, —OH, oxo, and C₁₋₃acyloxy;
b.) C₂₋₆ alkyl, wherein said C₂₋₆alkyl of R^{h2} is unsubstituted or substituted by 1 to 3 moieties independently selected from the group consisting of —OH, —N(R^{e})₂, —N(H)C(O)—C₁₋₃alkyl, and —N(H)C(O)—CH₂-phenyl;
c.) a group of the formula 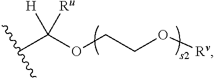
wherein:
(i) R^{u} is H or C₁₋₃alkyl;
(ii) the subscript s2 is 0, 1, 2, 3, 4, or 5; and
(iii) R^{v} is as set forth above;
F. a group of the formula 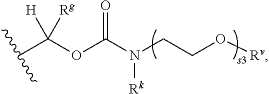
wherein
1. R^{k} is H or C₁₋₃alkyl;
2. the subscript s3 is 1, 2, 3, 4, or 5;
3. R^{g} and R^{v} are as set forth above;
G. a group of the formula 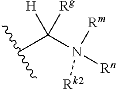
1. R^{g} is as set forth above;
2. R^{k2} is present or absent, and if present is H, C₁₋₈alkyl, —(CH₂)ₓ—CN, or C₃₋₆cycloalkyl;
3. R^{m} and Rⁿ are independently:
a) C₁₋₈alkyl,
b) —(CH₂)ₓ—CN,
c) —(CH₂)ₓ—N(R^{e})₂,
d) —(CH₂)₂O—(CH₂)₂N(R^{e})₂,
e) —(CH₂)ₓ—C(O)₂—C₁₋₃alkyl,
f) —Y³—C^{C}, wherein:
(i) Y³ is a bond, C₁₋₄alkylene, or —(CH₂)₂O—C(O)—; and
(ii) C^{C} is a cyclic moiety selected from the group consisting of C₃₋₆cycloalkyl, phenyl, or pyridyl; or
g) R^{m} and Rⁿ together with the N atom to which they are attached form a 5- to 6-membered heterocyclyl or heteroaryl containing 1 to 4 heteroatoms selected from the group consisting of N and O, wherein said heterocyclyl or heteroaryl is unsubstituted or is substituted by 1 to 3 moieties selected from the group consisting of C₁₋₃alkyl, —C(O)—C₁₋₃alkyl, —C(O)—N(R^{e})₂, fluoro, —C₁₋₃alkyl-OH, and —OH.
H. a group of the formula 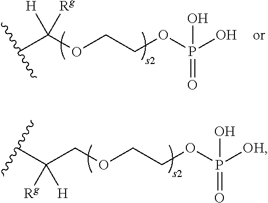
wherein R^{g} and the subscript s2 are as set forth above;
each R^{e} is independently H or C₁₋₄alkyl; and
each occurrence of the subscript x is independently 1, 2, or 3; and
wherein the compound is other than one of the following compounds or a pharmaceutically acceptable salt thereof:
methyl 4-[(6-{[4-(difluoromethyl)pyridin-2-yl]amino}-4-methyl-2,3′-bipyridin-6′-yl)-1-hydroxyethyl]cyclohexanecarboxylate;
ethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-hydroxyethyl 4-[1-(6-{[4-(difluoromethyl)pyridin-2-yl]amino}-4-methyl-2,3′-bipyridin-6′-yl)-1-hydroxyethyl]cyclohexanecarboxylate;
benzyl 4-[1-(6-{[4-(difluoromethyl)pyridin-2-yl]amino}-4-methyl-2,3′-bipyridin-6′-yl)-1-hydroxyethyl]cyclohexanecarboxylate;
propyl 4-[1-(6-{[4-(difluoromethyl)pyridin-2-yl]amino}-4-methyl-2,3′-bipyridin-6′-yl)-1-hydroxyethyl]cyclohexanecarboxylate;
propan-2-yl 4-[1-(6-{[4-(difluoromethyl)pyridin-2-yl]amino}-4-methyl-2,3′-bipyridin-6′-yl)-1-hydroxyethyl]cyclohexanecarboxylate;
butyl 4-[1-(6-{[4-(difluoromethyl)pyridin-2-yl]amino}-4-methyl-2,3′-bipyridin-6′-yl)-1-hydroxyethyl]cyclohexanecarboxylate
butan-2-yl 4-[1-(6-{[4-(difluoromethyl)pyridin-2-yl]amino}-4-methyl-2,3′-bipyridin-6′-yl)-1-hydroxyethyl]cyclohexanecarboxylate;
2-methylpropyl 4-[1-(6-{[4-(difluoromethyl)pyridin-2-yl]amino}-4-methyl-2,3′-bipyridin-6′-yl)-1-hydroxyethyl]cyclohexanecarboxylate;
pentyl 4-[1-(6-{[4-(difluoromethyl)pyridin-2-yl]amino}-4-methyl-2,3′-bipyridin-6′-yl)-1-hydroxyethyl]cyclohexanecarboxylate;
2,2-dimethylpropyl 4-[1-(6-{[4-(difluoromethyl)pyridin-2-yl]amino}-4-methyl-2,3′-bipyridin-6′-yl)-1-hydroxyethyl]cyclohexanecarboxylate;
hexyl 4-[−1-(6-{[4-(difluoromethyl)pyridin-2-yl]amino}-4-methyl-2,3′-bipyridin-6′-yl)-1-hydroxyethyl]cyclohexanecarboxylate;
heptyl 4-[1-(6-{[4-(difluoromethyl)pyridin-2-yl]amino}-4-methyl-2,3′-bipyridin-6′-yl)-1-hydroxyethyl]cyclohexanecarboxylate;
octyl 4-[1-(6-{[4-(difluoromethyl)pyridin-2-yl]amino}-4-methyl-2,3′-bipyridin-6′-yl)-1-hydroxyethyl]cyclohexanecarboxylate;
cyclohexyl 4-[1-(6-{[4-(difluoromethyl)pyridin-2-yl]amino}-4-methyl-2,3′-bipyridin-6′-yl)-1-hydroxyethyl]cyclohexanecarboxylate;
tetrahydro-2H-pyran-4-yl 4-[1-(6-{[4-(difluoromethyl)pyridin-2-yl]amino}-4-methyl-2,3-bipyridin-6′-yl)-1-hydroxyethyl]cyclohexanecarboxylate;
cyclohexylmethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-methoxyethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-(2-ethoxyethoxyl)ethyl 4-[−1-(6-{[4-(difluoromethyl)pyridin-2-yl]amino}-4-methyl-2,3-bipyridin-6′-yl)-1-hydroxyethyl]cyclohexanecarboxylate;
2-methoxy-2-oxoethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-(dimethylamino)-2-oxoethyl 4-[1-(6-{[4-(difluoromethyl)pyridin-2-yl]amino}-4-methyl-2,3′-bipyridin-6′-yl)-1-hydroxyethyl]cyclohexanecarboxylate;
2-(morpholin-4-yl)ethyl 4-[1-(6-{[4-(difluoromethyl)pyridin-2-yl]amino}-4-methyl-2,3-bipyridin-6′-yl)-1-hydroxyethyl]cyclohexanecarboxylate;
2-(dimethylamino)ethyl 4-[1-(6-{[4-(difluoromethyl)pyridin-2-yl]amino}-4-methyl-2,3-bipyridin-6′-yl)-1-hydroxyethyl]cyclohexanecarboxylate;
(5-methyl-2-oxo-1,3-dioxol-4-yl)methyl 4-[1-(6-{[4-(difluoromethyl)pyridin-2-yl]amino}-4-methyl-2,3′-bipyridin-6′-yl)-1-hydroxyethyl]cyclohexanecarboxylate;
[(ethoxycarbonyl)oxy]methyl 4-[1-(6-{[4-(difluoromethyl)pyridin-2-yl]amino}-4-methyl-2,3′-bipyridin-6′-yl)-1-hydroxyethyl]cyclohexanecarboxylate;
{[(propan-2-yloxy)carbonyl]oxy}methyl 4-[1-(6-{[4-(difluoromethyl)pyridin-2-yl]amino}-4-methyl-2,3′-bipyridin-6′-yl)-1-hydroxyethyl]cyclohexanecarboxylate;
1-[(ethoxycarbonyl)oxy]ethyl 4-[1-(6-{[4-(difluoromethyl)pyridin-2-yl]amino}-4-methyl-2,3′-bipyridin-6′-yl)-1-hydroxyethyl]cyclohexanecarboxylate;
1-{[(propan-2-yloxy)carbonyl]oxy}ethyl 4-[1-(6-{[4-(difluoromethyl)pyridin-2-yl]amino}-4-methyl-2,3′-bipyridin-6′-yl)-1-hydroxyethyl]cyclohexanecarboxylate;
1-{[(cyclohexyloxy)carbonyl]oxy}ethyl 4-[1-(6-{[4-(difluoromethyl)pyridin-2-yl]amino}-4-methyl-2,3′-bipyridin-6′-yl)-1-hydroxyethyl]cyclohexanecarboxylate;
(acetyloxy)methyl 4-[1-(6-{[4-(difluoromethyl)pyridin-2-yl]amino}-4-methyl-2,3′-bipyridin-6′-yl)-1-hydroxyethyl]cyclohexanecarboxylate;
[(2,2-dimethylpropanoyl)oxy]methyl 4-[1-(6-{[4-(difluoromethyl)pyridin-2-yl]amino}-4-methyl-2,3′-bipyridin-6′-yl)-1-hydroxyethyl]cyclohexanecarboxylate;
1-(acetyloxy)ethyl 4-[1-(6-{[4-(difluoromethyl)pyridin-2-yl]amino}-4-methyl-2,3′-bipyridin-6′-yl)-1-hydroxyethyl]cyclohexanecarboxylate;
1-[(2-methylpropanoyl)oxy]ethyl 4-[1-(6-{[4-(difluoromethyl)pyridin-2-yl]amino}-4-methyl-2,3′-bipyridin-6′-yl)-1-hydroxyethyl]cyclohexanecarboxylate; and
2-(acetyloxy)ethyl 4-[1-(6-{[4-(difluoromethyl)pyridin-2-yl]amino}-4-methyl-2,3′-bipyridin-6′-yl)-1-hydroxyethyl]cyclohexanecarboxylate.

2. The compound of claim 1 or a pharmaceutically acceptable salt thereof, wherein R^{a} is C₁₋₃alkyl substituted by 1 to 3 moieties selected from the group consisting of —OH, —N(R^{e})₂, —N(C₁₋₄alkyl)₃, —C(O)N(R^{e})₂, —S—(C₁₋₄ alkyl), —S(O)—(C₁₋₄ alkyl), and —S(O)₂—(C₁₋₄ alkyl).

3. The compound of claim 1 or a pharmaceutically acceptable salt thereof, wherein R^{a} is C₄₋₈alkyl, wherein said C₄₋₈alkyl is unsubstituted or substituted by 1-3 moieties selected from the group consisting of —OH, —N(R^{e})₂, —N(C₁₋₄alkyl)₃, —C(O)N(R^{e})₂, —S—(C₁₋₄ alkyl), —S(O)—(C₁₋₄ alkyl), and —S(O)₂—(C₁₋₄ alkyl).

4. The compound of claim 1 or a pharmaceutically acceptable salt thereof, wherein R^{a} is the group of the formula -M-R^{CH}.

5. The compound of claim 1 or a pharmaceutically acceptable salt thereof, wherein R^{a} is the group of the formula

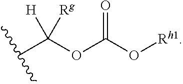

6. The compound of claim 1 or a pharmaceutically acceptable salt thereof, wherein R^{a} is the group of the formula

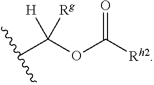

7. The compound of claim 1 or a pharmaceutically acceptable salt thereof, wherein R^{a} is the group of the formula

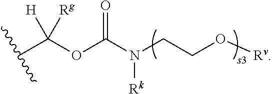

8. The compound of claim 1 or a pharmaceutically acceptable salt thereof, wherein R^{a} is the group of the formula

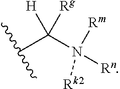

9. The compound of claim 1 or a pharmaceutically acceptable salt thereof, wherein R^{a} is the group of the formula

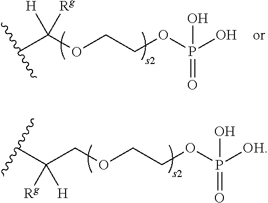

10. The compound of claim 1 selected from the group consisting of:
2-hydroxy-2-methylpropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
cis-4-aminocyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(2S)-2,3-dihydroxypropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
6-O-({trans-4-[(1R)-1-(6-{[4-(difluoromethyl)pyridin-2-yl]amino)-4-methyl-2,3′-bipyridin-6′-yl)-1-hydroxyethyl]cyclohexyl}carbonyl)-D-glucopyranose;
trans-3-aminocyclobutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
phenyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-(methylsulfanyl)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-(methylsulfonyl)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
3-(methylsulfanyl)propyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]-N,N,N-trimethylethanaminium;
(1-methyl-1H-imidazol-2-yl)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-hydroxy-3-methylbutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-(dimethylamino)propyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(4R)-2,2-dimethyl-1,3-dioxolan-4-yl]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(4S)-2,2-dimethyl-1,3-dioxolan-4-yl]methyl trans 4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(2R)-2,3-dihydroxypropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
piperidin-4-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
8-azabicyclo[3.2.1]oct-3-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
trans-3-aminocyclobutyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
tetrahydro-2H-pyran-4-ylmethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
piperidin-4-ylmethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-(methylamino)propyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
trans-4-aminocyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
trans-4-(methylamino)cyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
6-(dimethylamino)hexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
methyl 6-O-({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)-alpha-D-glucopyranoside;
3-(dimethylamino)-3-oxopropyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
4-methoxyphenyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
4-cyanophenyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[(6-aminohexyl)oxy]carbonyl})oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-oxo-2,4,7,10-tetraoxadodec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[2-(benzyloxy)ethoxy]carbonyl})oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
15-hydroxy-3-oxo-2,4,7,10,13-pentaoxapentadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-oxo-2,4,7,10,13-pentaoxapentadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2-ethoxyethoxy)carbonyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({[6-(dimethylamino)hexyl]oxy}carbonyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[2-ethoxy-1-(ethoxymethyl)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(tetrahydro-2H-pyran-4-ylmethoxy)carbonyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
({[2-(methylsulfanyl)ethoxy]carbonyl})oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[(trans-4-aminocyclohexyl)oxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[3-(dimethylamino)-3-oxopropoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(({[(1-methyl-1H-imidazol-2-yl)methoxy]carbonyl})oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
1-methyl-3-oxo-2,4,7,10-tetraoxadodec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
1-methyl-3-oxo-2,4,7,10-tetraoxadodec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
1-(1-methylethyl)-3-oxo-2,4,7,10-tetraoxadodec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}) carbonyl)oxy]methyl piperidine-4-carboxylate;
1-[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]ethyl 1-methyl-1H-pyrazole-4-carboxylate;
[(2-hydroxy-2-methylpropanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(3-hydroxy-3-methylbutanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(hydroxyacetyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2S)-2-hydroxy-3-(4-hydroxyphenyl)propanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl N-acetyl-L-leucinate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 1-methyl-1H-pyrazole-4-carboxylate;
[(3-hydroxypropanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl (3 alpha,7alpha, 12alpha)-3,7,12-trihydroxycholan-24-oate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl (3 alpha,7alpha)-3,7-dihydroxycholan-24-oate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl benzoate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl pyridine-3-carboxylate;
{[3-(3-hydroxyphenyl)propanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 5-oxo-D-prolinate;
{[(2S)-2-hydroxy-3-methylbutanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2R)-2-hydroxypropanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[3-(3,4-dihydroxyphenyl)propanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2R)-2-hydroxy-2-phenylacetyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 4-hydroxybenzoate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 2-(acetyloxy)benzoate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 2-hydroxybenzoate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl 2,5-dihydroxybenzoate;
[(3-hydroxybutanoyl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(1-hydroxycyclobutyl)carbonyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2S)-2-hydroxy-4-methylpentanoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[(N-acetylseryl)oxy]methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl N˜2˜N˜6˜-bis[(benzyloxy)carbonyl]-L-lysinate;
1-{[(2-methoxyethyl)(methyl)carbamoyl]oxy}ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
{[(2-methoxyethyl)(methyl)carbamoyl]oxy}methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
3-oxo-2,7,10-trioxa-4-azaundec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
4-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl})-4-methylmorpholin-4-ium iodide;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl})-N,N-dimethylpropan-2-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl)}carbonyl)oxy]methyl}-N-ethyl-N-(1-methylethyl)propan-2-aminium;
N,N-dibutyl-N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}butan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-2-(dimethylamino)-N,N-dimethylethanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-diethylbutan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-diethyl-4-hydroxypentan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dioctyloctan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}) carbonyl)oxy]methyl})-N,N-dipropylpropan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-diethylethanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dimethylcyclohexanaminium;
4-(diethylcarbamoyl)-1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-1-methylpiperazin-1-ium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}) carbonyl)oxy]methyl})-4,4-difluoro-1-methylpiperidinium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}) carbonyl)oxy]methyl}-1-ethylpiperidinium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl})-N,N-dimethyl-1-phenylethanaminium;
1-(cyanomethyl)-1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}piperidinium;
4-acetyl-1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-1-methylpiperidinium;
N-butyl-N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N-methylbutan-1-aminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-2-ethoxy-N,N-dimethyl-2-oxoethanaminium;
N-benzyl[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]-N,N-dimethylmethanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-dimethyl-2-pyridin-2-ylethanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N,N-diethylcyclohexanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}) carbonyl)oxy]methyl})-2-[2-(dimethylamino)ethoxy]-N,N-dimethylethanaminium;
N-cyclohexyl-N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-N-methylcyclohexanaminium;
N-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}) carbonyl)oxy]methyl})-N,N-dimethyl-2-[(phenylcarbonyl)oxy]ethanaminium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}) carbonyl)oxy]methyl}-1,4-dimethylpiperazin-1-ium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-4-hydroxy-1-methylpiperidinium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-4-(2-hydroxyethyl)-1-methylpiperazin-1-ium;
1-{[({trans-4-[(1R)-1-(6-{[4-(difluoromethyl)pyridin-2-yl]amino}-4-methyl-2,3′-bipyridin-6′-yl)-1-hydroxyethyl]cyclohexyl}carbonyl)oxy]methyl}-4-(hydroxymethyl)-1-methylpiperidinium;
1-{[({trans-4-[(1R)-1-(6-{[4-(Difluoromethyl)pyridin-2-yl]amino}-4-methyl-2,3′-bipyridin-6′-yl)-1-hydroxyethyl]cyclohexyl}carbonyl)oxy]methyl}-3-methyl-1H-imidazol-3-ium;
(5-methyl-1H-tetrazol-1-yl)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(5-methyl-2H-tetrazol-2-yl)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
1H-imidazol-1-ylmethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-[(R)-methylsulfinyl]ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-[(S)-methylsulfinyl]ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[3-(methylsulfonyl)propoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-(methylsulfinyl)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
3-(methylsulfinyl)propyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
({[2-(methylsulfinyl)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(({[2-(methylsulfinyl)ethoxy]carbonyl}oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
(([2-(methylsulfonyl)ethoxy]carbonyl)oxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
17,17-dihydroxy-17-oxido-3-oxo-2,4,7,10,13,16-hexaoxa-17λ⁵-phosphaheptadec-1-yl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate;
2-(phosphonooxy)ethyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
trans-4-(dimethylamino)cyclohexyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate; and
(phosphonooxy)methyl trans-4-[(1R)-1-(6-{[4-(difluoromethyl)pyridin-2-yl]amino}-4-methyl-2,3′-bipyridin-6′-yl)-1-hydroxyethyl]cyclohexanecarboxylate
or a pharmaceutically acceptable salt thereof.

11. A pharmaceutical composition comprising a therapeutically effective amount of a compound of claim 1 or a pharmaceutically acceptable salt thereof, and a pharmaceutically acceptable carrier.

12. A method for the treatment or prevention of a Syk-mediated disease which comprises administering to a patient in need thereof a therapeutically effective amount of a compound of claim 1 or a pharmaceutically acceptable salt thereof.

13. A method of claim 12 wherein said Syk-mediated disease is selected from the group consisting of rheumatoid arthritis, asthma, and systemic lupus erythematosis.

14. (canceled)

15. (canceled)
